SEQUENCE LISTING (130 sequences)

SEQ ID NO: 1 - DNA, 1399 bp, Bacillus subtilis
  gene (1)..(1399): acoA
attgtcaaaggccgggtgatatccggtcttttttttgcatgctgtaaaacgagacaaatgaatcagtttgagacaaaacgagacacacgtctcaaactgtctccaaagtgaagatgagaagactgattttacgggctcaaaagactggcacacttcttgcatttataatggtgaaccctaaatagaaggaggcgcacaaaatgaaattgttaaaacgagaaggcttgtcattaactgaggaaaaagcgctgtggatgtaccaaaagatgctggagatcaggggctttgaagacaaagtgcatgaactgttcgcccagggagtgcttcccggattcgttcatttatatgccggtgaggaagccgtggctgtaggggtgtgcgctcatttacatgatggcgacagcattacaagcacccacaggggacatggacattgtatcgccaaaggctgtgacctggacggcatgatggcggaaattttcgggaaagcgaccggattgtgcaaaggcaagggcggttctatgcacattgcggatcttgataaaggcatgttaggcgcaaatggaatcgtcgggggcggctttacgctcgcatgcggatcagcgctcacggctaaatataaacagactaaaaatgtaagcgtttgctttttcggggacggggcaaataaccaaggtaccttccacgaagggctgaatttagcggctgtatggaaccttcctgtcgtatttgttgctgaaaacaacggctatggcgaagctaccccatttgagtacgcatcagcctgtgattcaatcgccgatcgggcggctgcttataacatgccgggggttacagttgacggcaaagatattttagcagtttaccaggcagccgaggaagcgatagaaagagcaagaaacggcggcggcccgtctttgattgaatgtatgacctacagaaactacggccatttcgaaggagatgcccaaacctataaaacgaaggatgaaagagttgagcaccttgaagaaaaagatgccattcaaggttttaaaaactaccttttaaaagaaacagatgctaataagctgtcagacattgaacagcgtgtcagcgaatcgattgaaaaagccgtctcgttcagcgaagacagcccatatccaaaagattcggagctgctgacagatgtgtatgtgtcatatgaaaaaggaggaatgtaagagatggcgagagtcataagcatgtcagacgcgatcaatgaagcaatgaagcttgcgatgagaaaagacgaaaatgtgcttttgatcggtgaggatgtcgccgggggagcggcggtcgatcatttgcaggatgatgaagcatggggcggtgtattaggggtcacaaagggactcgtacaggaattcgggcgtacaag

SEQ ID NO: 2 - protein, 333 aa, Bacillus subtilis
MKLLKREGLSLTEEKALWMYQKMLEIRGFEDKVHELFAQGVLPGFVHLYAGEEAVAVGVCAHLHDGDSITSTHRGHGHCIAKGCDLDGMMAEIFGKATGLCKGKGGSMHIADLDKGMLGANGIVGGGFTLACGSALTAKYKQTKNVSVCFFGDGANNQGTFHEGLNLAAVWNLPVVFVAENNGYGEATPFEYASACDSIADRAAAYNMPGVTVDGKDILAVYQAAEEAIERARNGGGPSLIECMTYRNYGHFEGDAQTYKTKDERVEHLEEKDAIQGFKNYLLKETDANKLSDIEQRVSESIEKAVSFSEDSPYPKDSELLTDVYVSYEKGGM

SEQ ID NO: 3 - DNA, 961 bp, Bacillus subtilis
  gene (1)..(961): ahpC
aaccttatctaattgagaatcagcctctcatttattataaaagaaatgcctaaatcctacctgtcacacctttattaagatgaaaaaaagtaggttatggcttgacaaaaaatatatattaattaataattcatatataattagaattattattgaaagcgattatgctttctaatacatttttaggaggaatatacattatgtctttaatcggtaaagaagtacttccattcgaagcaaaagcattcaaaaacggtgaattcatcgatgtaacaaacgaagatttgaaaggccaatggagcgtattctgcttctacccagcagatttctctttcgtatgcccaactgagcttgaagatcttcaagaacaatatgctgcacttaaagaattaggtgttgaagtatactctgtttctacagatactcacttcgtacacaaaggctggcatgacagctctgaaaaaatcagcaaaatcacttacgcaatgatcggtgacccatctcaaacgatctctcgcaacttcgatgttcttgacgaagaaactggccttgctgaccgcggaacattcatcatcgatccagacggcgttatccaaactgttgaaatcaatgcaggcggtatcggccgtgacgcaagcaaccttgtaaacaaagtaaaagcagctcaatacgttcgtcaaaacccaggtgaagtttgcccggctaaatgggaagaaggcggcgaaactcttacacctagccttgatctagtaggtaaaatctaaggagtgcattcaattggtacttgatgcaaatatcaaagcacaattaaatcaatatatgcagctaattgagaatgacattgttctcaaagttagcgcaggcgaagatgacacttctaaggacatgctggctctcgttgatgagctggcttccatgtcatcgaaaatttcagttgaaaaagctgaattaaacagaacgc

SEQ ID NO: 4 - protein, 187 aa, Bacillus subtilis
MSLIGKEVLPFEAKAFKNGEFIDVTNEDLKGQWSVFCFYPADFSFVCPTELEDLQEQYAALKELGVEVYSVSTDTHFVHKGWHDSSEKISKITYAMIGDPSQTISRNFDVLDEETGLADRGTFIIDPDGVIQTVEINAGGIGRDASNLVNKVKAAQYVRQNPGEVCPAKWEEGGETLTPSLDLVGKI

SEQ ID NO: 5 - DNA, 1927 bp, Bacillus subtilis
  gene (1)..(1927): ahpF
tccagacggcgttatccaaactgttgaaatcaatgcaggcggtatcggccgtgacgcaagcaaccttgtaaacaaagtaaaagcagctcaatacgttcgtcaaaacccaggtgaagtttgcccggctaaatgggaagaaggcggcgaaactcttacacctagccttgatctagtaggtaaaatctaaggagtgcattcaattggtacttgatgcaaatatcaaagcacaattaaatcaatatatgcagctaattgagaatgacattgttctcaaagttagcgcaggcgaagatgacacttctaaggacatgctggctctcgttgatgagctggcttccatgtcatcgaaaatttcagttgaaaaagctgaattaaacagaacgccgagcttcagtgtcaatcgtgtcggagaagacactggcgtgactttcgccggtatccctctgggccacgaattcacatcattagtgttggcgttgctccaagtgagcggcagaccgcctaaggtagaccaaaaagtcattgatcaggtgaagaagatcagtggtgaataccacttcgaatcttatatcagcctgacatgccacaactgtcctgatgttgtacaagctttaaacatgatgagcgtgctgaacccgaacattacgcacacgatgattgacggtgcagcatacaaagcagaagttgaaagcaaaaacatcatggcagtgccgaccgtttacctgaatggcgaatccttcggaagcggccgtatgacgcttgaagaaattcttgcgaaaatgggcagcggcacagatgcatctgagtttgctgacaaagagccgtttgacgttcttgtggtcggaggcggacctgctggtgcaagtgcagcgatctacactgcacgtaaaggcatccgaactggtgttgtcgctgagcgcttcggcggacaggttctcgacaccatgagcatcgaaaacttcatcagcgtcaaagcgacggaaggaccgaagctagcggcaagtcttgaagagcatgtgaaggaatatgatattgatgtcatgaaccttcagcgtgcgaaacgccttgagaagaaagatctgttcgaacttgaactcgaaaacggcgctgtcctgaaaagtaaaacagtgatcctttcaacaggtgcacgctggcgcaatgtcaacgtccccggtgaacaagagttcaaaaacaaaggtgtcgcatactgcccgcactgtgatgggccattgtttgaaggcaaagacgttgcggtaatcggcggcggaaactctggtatcgaagcagcgattgatcttgcaggtattgtcaatcacgttactgtactagaattcgcgccggaactgaaagcagacgaagtcctgcaaaaacgtctctacagcctgcctaacgttactgtcgtgaaaaacgcacaaacaaaagaaatcacaggtgatcagagtgttaacggcatcacatatgtagaccgcgaaacaggcgaagaaaaacacgttgaacttcaaggtgtattcgtccaaatcggtctcgtgccaaacacagaatggttagaaggaacggttgaacgcaaccgcatgggcgaaattattgtcgacaaacacggcgcaacaagcgtaccaggcttatttgctgccggcgactgcacagacagtgcgtacaaccaaattattatttctatgggatcaggtgcaactgccgccctcggcgcgtttgattacctaatccgtaactaatataagaaatccgctatattgccagattggcaggatagcggatttttctttttctacagaaaaaaaccgaaccaggcactacggtaaaagtgctcgattcgagcgcttcttatagttcctcaccattcgtttgaatgacatttttataccactcaaaggagtctttcttatatcgtttcatggatccgtttccttca

SEQ ID NO: 6 - protein, 509 aa, Bacillus subtilis
LVLDANIKAQLNQYMQLIENDIVLKVSAGEDDTSKDMLALVDELASMSSKISVEKAELNRTPSFSVNRVGEDTGVTFAGIPLGHEFTSLVLALLQVSGRPPKVDQKVIDQVKKISGEYHFESYISLTCHNCPDVVQALNMMSVLNPNITHTMIDGAAYKAEVESKNIMAVPTVYLNGESFGSGRMTLEEILAKMGSGTDASEFADKEPFDVLVVGGGPAGASAAIYTARKGIRTGVVAERFGGQVLDTMSIENFISVKATEGPKLAASLEEHVKEYDIDVMNLQRAKRLEKKDLFELELENGAVLKSKTVILSTGARWRNVNVPGEQEFKNKGVAYCPHCDGPLFEGKDVAVIGGGNSGIEAAIDLAGIVNHVTVLEFAPELKADEVLQKRLYSLPNVTVVKNAQTKEITGDQSVNGITYVDRETGEEKHVELQGVFVQIGLVPNTEWLEGTVERNRMGEIIVDKHGATSVPGLFAAGDCTDSAYNQIIISMGSGATAALGAFDYLIRN

SEQ ID NO: 7 - DNA, 3127 bp, Bacillus subtilis
  gene (1)..(3127): citB
tcttttattgtgttatatgagagagcgtaaacagtaaaaattaccggtacatttttctcataagtcgaacttattgtatttaataaaaacattgatatttacttatgtatgattttgttttaatatgaaattgtgagaaaattgtgatggaatgtaatgttcatctgagaatgatctgatctgaagggggattttggagaatggcaaacgagcaaaaaactgcagcaaaagacgttttccaagcgagaaaaacgtttactacaaatgggaaaacatatcattactactctttaaaagcgttagaagattcaggtataggaaaggtttcgaagcttccttattccatcaaagttcttttagaatcagtattgcgtcaagttgacggcttcgttatcaaaaaagaacacgtggaaaatttggcaaaatggggaactgccgaattaaaggatatcgacgttccgttcaaaccgtctcgtgttattttacaagacttcacaggggttccggcagtagtagatctggcttcactgcgtaaagcaatggcagctgtcggcggagatcctgataaaatcaaccctgaaattcctgttgatctcgttatcgatcactctgtacaggtagataaagcgggtacagaagatgcattagcagtaaatatggacttggaattcgaaagaaatgcagagcgctacaaatttttaagctgggcaaagaaagcgtttaacaattatcaggcagtaccgcctgcaacaggtattgtgcaccaggtcaaccttgagttcttggcaagtgttgtccatgccattgaagaagacggcgagcttgtaacgtatcctgatacgcttgtcggaacagactcacacacaacaatgattaacggtatcggtgttctcggctggggtgtcggtggaattgaagctgaagcgggaatgcttggacagccttcttacttcccagttccagaggtaatcggcgcgaaacttgtcggcaagcttccaaacggaacaacagctactgacttggcgttaaaagtaacacaagtgctgcgtgaaaaaggcgttgtcggtaaatttgttgaattcttcggaccgggaattgctgaactgccgcttgcagatcgcgcaacaattgcgaatatggctccggaatacggtgctacatgcggattcttcccagtagatgaagaagcgcttaactacctgcgcctgactggccgtgatcctgaacatattgatgttgttgaagcatactgcagaagcaatggcttgttctacactccagatgcggaagaccctcaatttactgatgtggttgaaattgacctgtctcaaattgaagcaaacttatcgggtccaaagcgtcctcaggatttgatcccgctatctgctatgcaggaaacgtttaaaaagcaattagtcagccctgcaggtaaccaaggattcggtttaaatgctgaggaagaaaataaagaaattaagtttaaactccttaacggcgaagaaacagttatgaaaacgggtgcgatcgccattgctgcgattacaagctgtacaaatacatcaaacccatacgtgctgatcggcgccggactggtagcgaaaaaagcggttgagttagggcttaaggtgcctaattacgtgaaaacgtcacttgcaccgggttctaaagttgttacaggatatcttgtgaattcaggccttcttccatacatgaaagagcttggctttaacctcgttgggtacggctgtacaacatgtatcgggaactcaggtccgctttcaccggaaatcgaagaagcggttgcgaaaaatgatcttctgattacgtctgtcctttccggaaaccgtaactttgaaggacgtattcacccgcttgttaaaggcaactatcttgcttcaccgccgcttgttgtggcatatgcgctggctggaacggtaaacattaacttaaaaaccgatccaatcggtgtgggcaaggatggtcaaaacgtatactttaatgatatttggccgtcaatggacgaaatcaatgcacttgttaagcaaactgttacgccagagctattccgcaaagagtatgaaacagtatttgatgacaacaagcgctggaacgaaattgaaacaacagatgaagctttatataaatgggataacgattcaacttacatccaaaacccaccattctttgaagagatgtctgttgagccaggcaaggttgagccattaaaaggactgcgtgttgtcggtaaattcggcgattcagtcacaactgaccatatttctcctgcgggggcaatcggaaaagatacgcctgccggaaagtatttgcaagaaaaaggtgtttcacctcgtgactttaactcctacggctcccgccgtggaaaccatgaagtcatgatgagaggaacatttgccaacattcgcatcaaaaaccaaatcgcaccgggtacagaaggcggatttacgacgtactggccgactggtgaagtaacatccatctatgatgcatgcatgaaatacaaagaagataaaaccggtcttgtcgtattagcaggaaaagactatggtatgggatcttcacgtgactgggctgcaaaaggaacaaaccttctcggcatcagaacggtcattgcagagagcttcgaaagaattcacagaagcaatcttgtattcatgggtgtgctgccgcttcagtttaaacaaggtgaaaatgcggatacactcggcttaacgggtaaagaagtcatcgaggtagatgttgatgaaacagttcgtcctcgtgaccttgtgactgtaagagcaatcaatgaagacggcaatgtaacaacttttgaagcagtcgtccgctttgatagtgaagttgaaattgattactaccgccatggcggcatccttcaaatggtgcttcgtgaaaaaatgaagcagtcctgatgaatcaataggaagagaaggcatttcgctttctcttctttttatgacacaatgtgctgcggaggtggcatatgctgaaaaaatggctcgcagggatcctgcttatcatgctagtcggttatacgggatggaatttatatcaaacatacagcaaaaaagaagtcggaattcaagaggggcaacaagcacctgatttt

SEQ ID NO: 8 - protein, 909 aa, Bacillus subtilis
MANEQKTAAKDVFQARKTFTTNGKTYHYYSLKALEDSGIGKVSKLPYSIKVLLESVLRQVDGFVIKKEHVENLAKWGTAELKDIDVPFKPSRVILQDFTGVPAVVDLASLRKAMAAVGGDPDKINPEIPVDLVIDHSVQVDKAGTEDALAVNMDLEFERNAERYKFLSWAKKAFNNYQAVPPATGIVHQVNLEFLASVVHAIEEDGELVTYPDTLVGTDSHTTMINGIGVLGWGVGGIEAEAGMLGQPSYFPVPEVIGAKLVGKLPNGTTATDLALKVTQVLREKGVVGKFVEFFGPGIAELPLADRATIANMAPEYGATCGFFPVDEEALNYLRLTGRDPEHIDVVEAYCRSNGLFYTPDAEDPQFTDVVEIDLSQIEANLSGPKRPQDLIPLSAMQETFKKQLVSPAGNQGFGLNAEEENKEIKFKLLNGEETVMKTGAIAIAAITSCTNTSNPYVLIGAGLVAKKAVELGLKVPNYVKTSLAPGSKVVTGYLVNSGLLPYMKELGFNLVGYGCTTCIGNSGPLSPEIEEAVAKNDLLITSVLSGNRNFEGRIHPLVKGNYLASPPLVVAYALAGTVNINLKTDPIGVGKDGQNVYFNDIWPSMDEINALVKQTVTPELFRKEYETVFDDNKRWNEIETTDEALYKWDNDSTYIQNPPFFEEMSVEPGKVEPLKGLRVVGKFGDSVTTDHISPAGAIGKDTPAGKYLQEKGVSPRDFNSYGSRRGNHEVMMRGTFANIRIKNQIAPGTEGGFTTYWPTGEVTSIYDACMKYKEDKTGLVVLAGKDYGMGSSRDWAAKGTNLLGIRTVIAESFERIHRSNLVFMGVLPLQFKQGENADTLGLTGKEVIEVDVDETVRPRDLVTVRAINEDGNVTTFEAVVRFDSEVEIDYYRHGGILQMVLREKMKQS

SEQ ID NO: 9 - DNA, 2830 bp, Bacillus subtilis
  gene (1)..(2830): clpC
ttaggcatagacttaggcataataaaaggactttcaagcaacatacttaacgaacttatgattttgacgcagccagggtttttacagcaatactcgggaggcgctttgcgaccgaacgaaagggacattcgaagagcggctctcatcagagaacggcttcacttagaaatgaatgggaaaagacaggaggatgaatcgatatgatgtttggaagatttacagaacgagctcaaaaagtactggcgctagcacaggaagaagcacttcggttaggtcataataacattggcactgagcatattttattaggactggtaagagaaggagagggcattgctgctaaagctcttcaagcgcttggactcggttcagaaaaaattcagaaagaagtagaaagtttgatcgggcgcgggcaggaaatgtctcaaacgattcattatactcctagagctaaaaaagtcattgagctttcaatggatgaggcaagaaaactcggtcattcttatgtgggaacagaacatattcttcttggtctgattcgtgaaggagaaggtgttgctgcgagagttctgaataatctcggtgtcagcttaaataaagcaagacagcaggtgctccagcttctaggaagtaatgaaacaggatcatcagcggcaggaacaaacagcaatgcgaatacgcctacgcttgacagcttggcaagagacttaactgctattgcgaaggaagacagccttgaccctgtaatcggcagaagcaaggagatccagcgtgtcattgaagtgttaagccgcagaacgaaaaacaaccctgttctcattggggaaccaggtgtaggtaaaacggctatcgcagaaggtttggcacagcaaattatcaataatgaagtacccgaaattttgcgtgataaacgtgtgatgacattagacatgggaacagttgttgccggcacaaaataccgcggagaatttgaggatcgcctgaagaaggtcatggatgaaattcgccaggcaggaaatatcattctattcatcgatgagctccatacattaatcggggcaggcggagcagaaggtgctattgatgcatctaatattttaaaaccttcacttgctcgtggcgaactccaatgtattggtgcaacgactcttgatgagtaccgtaaatatattgaaaaagatgcagcactggaacgccgttttcagccgattcaggttgatcagccatctgtagatgaaagtattcaaattttacaaggtctgcgtgacagatacgaagcccaccaccgcgtttctatcactgatgatgccattgaagctgcggttaagctttctgacagatatatttctgaccgcttccttccggataaagcaattgacttgatcgatgaagcgggttcaaaggtgagactgcgctcatttacaacgcctcctaacttaaaagagcttgagcagaagcttgatgaggttcgtaaagagaaggatgcggcagtgcaaagccaagagtttgaaaaagctgcttccttgcgtgatactgaacaacgcctgcgcgagcaagtagaggatacgaagaaatcatggaaagagaagcaagggcaggaaaactcagaggttactgtggatgatattgcgatggttgtatccagctggaccggtgtgcctgtatctaaaatcgcccaaactgaaactgataagcttctcaatatggaaaacattcttcactcccgtgtcatcggccaggatgaagcagttgtagcggttgcaaaagccgtcagacgtgcaagagcaggattgaaagatcctaaacgcccaatcggctcattcattttcttaggccctacaggtgtaggtaaaacagaacttgcacgagcacttgctgaatccatttttggcgatgaagaatccatgatcagaattgatatgtctgaatacatggaaaaacattcaacttcaagacttgttggttcacctccgggttatgtgggatatgatgaaggcggtcaattgacagagaaagtcagaagaaaaccttactctgtcgtgcttcttgatgagatcgagaaagcgcaccctgatgtcttcaatatccttctgcaagttcttgaagacggacgattgactgattctaaaggacgcacagtcgatttccgcaataccattctgatcatgacatcaaacgtcggagcaagtgagctaaaacgcaataaatatgtcggctttaacgttcaggatgaaactcaaaatcataaagacatgaaagataaagtgatgggtgaattaaaacgagcgttcagacctgagttcatcaaccgtattgatgaaatcattgtcttccattcacttgagaaaaaacatctcactgagattgtgtcattaatgtctgatcaattaacgaaacgcctgaaagaacaagatctttctatcgaattgacagatgctgcaaaagcgaaagtcgcggaagagggcgttgacctggaatacggtgcccgtccgttaagaagagcgatccaaaaacatgtcgaggatcgtttatctgaagaactcctcagaggaaatattcataaaggacagcatattgttcttgatgtagaagatggcgaatttgtcgtaaaaacgactgctaaaacgaattaatatagaagacggaaatgaggcatacagcatgtaagtgtatgcctcactttcatatcaagaaatagaacaagggagaggtcttacactatatatggctaaaacaaaatcgaagttcatctgccaatcctgcggctacgagtctccaaaatggatggggaaatgtccgggctgtggtgcttggaatacaatggtggaag

SEQ ID NO: 10 - protein, 810 aa, Bacillus subtilis
MMFGRFTERAQKVLALAQEEALRLGHNNIGTEHILLGLVREGEGIAAKALQALGLGSEKIQKEVESLIGRGQEMSQTIHYTPRAKKVIELSMDEARKLGHSYVGTEHILLGLIREGEGVAARVLNNLGVSLNKARQQVLQLLGSNETGSSAAGTNSNANTPTLDSLARDLTAIAKEDSLDPVIGRSKEIQRVIEVLSRRTKNNPVLIGEPGVGKTAIAEGLAQQIINNEVPEILRDKRVMTLDMGTVVAGTKYRGEFEDRLKKVMDEIRQAGNIILFIDELHTLIGAGGAEGAIDASNILKPSLARGELQCIGATTLDEYRKYIEKDAALERRFQPIQVDQPSVDESIQILQGLRDRYEAHHRVSITDDAIEAAVKLSDRYISDRFLPDKAIDLIDEAGSKVRLRSFTTPPNLKELEQKLDEVRKEKDAAVQSQEFEKAASLRDTEQRLREQVEDTKKSWKEKQGQENSEVTVDDIAMVVSSWTGVPVSKIAQTETDKLLNMENILHSRVIGQDEAVVAVAKAVRRARAGLKDPKRPIGSFIFLGPTGVGKTELARALAESIFGDEESMIRIDMSEYMEKHSTSRLVGSPPGYVGYDEGGQLTEKVRRKPYSVVLLDEIEKAHPDVFNILLQVLEDGRLTDSKGRTVDFRNTILIMTSNVGASELKRNKYVGFNVQDETQNHKDMKDKVMGELKRAFRPEFINRIDEIIVFHSLEKKHLTEIVSLMSDQLTKRLKEQDLSIELTDAAKAKVAEEGVDLEYGARPLRRAIQKHVEDRLSEELLRGNIHKGQHIVLDVEDGEFVVKTTAKTN

SEQ ID NO: 11 - DNA, 991 bp, Bacillus subtilis
  gene (1)..(991): clpP
tatttcgagaggccgttttttaaatagacaaagcatcttaattggcaatctgacatacgatggtttgaacccctgtatttttggggaaaatgggaaaaagagacgactttgcatatcaggccagctttttgtttgacctttattgaccaaaaatgtatcatgtaactacatacttacctaaaaggtgaaggaggagcattatgaatttaatacctacagtcattgaacaaacgaaccgcggggaaagagcgtatgacatttattctcgtctattaaaggatcgtatcatcatgcttggatctgcgattgatgacaacgttgcgaactccatcgtgtcacagcttttattcttagcagcagaagaccctgaaaaagaaatttctctttacatcaacagcccgggcggctctattacagccggtatggcgatctatgataccatgcagtttattaagccgaaggtatctacaatttgtatcggtatggctgcgtcaatgggcgcgttcctgcttgcagccggcgaaaaaggcaaacgctatgcgcttccaaacagtgaagtcatgattcaccagcctcttggcggtgcgcaaggtcaagcgacagaaattgaaattgctgcgaaacgcattctcttgcttcgcgacaaattaaacaaagtcctagctgaacgtactggccagccgcttgaagtgatcgaacgcgacacagaccgtgataacttcaagtctgctgaagaagcgcttgaatacggcctgattgacaaaattttgactcacacagaagacaaaaagtaataacacaacctgcaagagctgcgtctcttgcaggtttttttcatttcaaggaattgactcccttattctatactgctcccattcctcatgtaacagctcaagcgtcaaatcactcgtctgcctcacaaccagatcagcaccaagcatcggctgtccttgaccgacaccaaccgcaaacatccccgcagatttgattg

SEQ ID NO: 12 - protein, 197 aa, Bacillus subtilis
MNLIPTVIEQTNRGERAYDIYSRLLKDRIIMLGSAIDDNVANSIVSQLLFLAAEDPEKEISLYINSPGGSITAGMAIYDTMQFIKPKVSTICIGMAASMGAFLLAAGEKGKRYALPNSEVMIHQPLGGAQGQATEIEIAAKRILLLRDKLNKVLAERTGQPLEVIERDTDRDNFKSAEEALEYGLIDKILTHTEDKK

SEQ ID NO: 13 - DNA, 1024 bp, Escherichia coli
  gene (1)..(1024): clpP
ccaggtggtgggcttttttttgtcatgaattttgcatggaaccgtgcgaaaagcctctttcggtgttagcgtaacaacaaaagattgttatgcttgaaatatggtgatgccgtacccataacacagggactagctgataatccgtccataaggttacaatcggtacagcaggttttttcaattttatccaggagacggaaatgtcatacagcggcgaacgagataactttgcaccccatatggcgctggtgccgatggtcattgaacagacctcacgcggtgagcgctcttttgatatctattctcgtctacttaaggaacgcgtcatttttctgactggccaggttgaagaccacatggctaacctgattgtggcgcagatgctgttcctggaagcggaaaacccagaaaaagatatctatctgtacattaactccccaggcggggtgatcactgccgggatgtctatctatgacaccatgcagtttatcaagcctgatgtcagcaccatctgtatgggccaggcggcctcgatgggcgctttcttgctgaccgcaggggcaaaaggtaaacgtttttgcctgccgaattcgcgcgtgatgattcaccaaccgttgggcggctaccagggccaggcgaccgatatcgaaattcatgcccgtgaaattctgaaagttaaagggcgcatgaatgaacttatggcgcttcatacgggtcaatcattagaacagattgaacgtgataccgagcgcgatcgcttcctttccgcccctgaagcggtggaatacggtctggtcgattcgattctgacccatcgtaattgatgccagaggcgcaactgtgccgctatacttatccagggcggcacaacgctgtaagcggcttgcgcctgagaatggcatttgcgtcgtcgtgtgcggcacaaagaacaaagaagaggttttgacccatgacagataaacgcaaagatggctcaggcaaattgctgtattgctctttttgcggcaaaagccagcatgaagtg

SEQ ID NO: 14 - protein, 207 aa, Escherichia coli
MSYSGERDNFAPHMALVPMVIEQTSRGERSFDIYSRLLKERVIFLTGQVEDHMANLIVAQMLFLEAENPEKDIYLYINSPGGVITAGMSIYDTMQFIKPDVSTICMGQAASMGAFLLTAGAKGKRFCLPNSRVMIHQPLGGYQGQATDIEIHAREILKVKGRMNELMALHTGQSLEQIERDTERDRFLSAPEAVEYGLVDSILTHRN

SEQ ID NO: 15 - DNA, 1177 bp, Bacillus subtilis
  gene (1)..(1177): codY
gtgcgagacgccttcatacaatacttgaacgcctattagaagatttgtcgtttgaagctccagatgtaacgatggagaaaataacgattacaccacagtatgtcgaagaaaagctcggaacgatagccaaaaacaaagatttaagtcaatttatattgtgaaaaatttaatatgaggaatgtttaggaggattatttatcatggctttattacaaaaaacaagaattattaactccatgctgcaagctgcggcagggaaaccggtaaacttcaaggaaatggcggagacgctgcgggatgtaattgattccaatattttcgttgtaagccgcagagggaaactccttgggtattcaattaaccagcaaattgaaaatgatcgtatgaaaaaaatgcttgaggatcgtcaattccctgaagaatatacgaaaaatctgtttaatgtccctgaaacatcttctaacttggatattaatagtgaatatactgctttccctgttgagaacagagacctgtttcaagctggtttaacaacaattgtgccgatcatcggaggcggggaaagattaggaacacttattctttcgcgtttacaagatcaattcaatgacgatgacttaattctagctgaatacggcgcaacagttgtcggaatggaaatcctaagagaaaaagcagaagaaattgaagaggaagcaagaagcaaagctgtcgtacaaatggctatcagctcgctttcttacagtgagcttgaagcaattgagcacatttttgaggagcttgacggaaatgaaggtcttcttgttgcaagtaaaattgctgaccgtgtcggcattacccgttctgttattgtgaacgcactcagaaagctggagagcgccggtgttatcgagtctagatcattaggaatgaaaggtacttatatcaaggtactaaacaacaaattcctaattgaattagaaaatctaaaatctcattaatcacaaaaagaaccctttttgagggttctttttttatttcaaataaaggaaatcaataagctttatttctcgggttgaaagtctttctatgtaataattttaataaattttgcatttttcttcaaaaagtttcaaaaatgccgaaaagaaaggagaaaaaacagaaattctgctattttcaggcttatatcagggcg

SEQ ID NO: 16 - protein, 259 aa, Bacillus subtilis
MALLQKTRIINSMLQAAAGKPVNFKEMAETLRDVIDSNIFVVSRRGKLLGYSINQQIENDRMKKMLEDRQFPEEYTKNLFNVPETSSNLDINSEYTAFPVENRDLFQAGLTTIVPIIGGGERLGTLILSRLQDQFNDDDLILAEYGATVVGMEILREKAEEIEEEARSKAVVQMAISSLSYSELEAIEHIFEELDGNEGLLVASKIADRVGITRSVIVNALRKLESAGVIESRSLGMKGTYIKVLNNKFLIELENLKSH

SEQ ID NO: 17 - DNA, 613 bp, Escherichia coli
  gene (1)..(613): cspA
attgttgcatcacccgccaatgcgtggcttaatgcacatcaacggtttgacgtacagaccattaaagcagtgtagtaaggcaagtcccttcaagagttatcgttgatacccctcgtagtgcacattcctttaacgcttcaaaatctgtaaagcacgccatatcgccgaaaggcacacttaattattaaaggtaatacactatgtccggtaaaatgactggtatcgtaaaatggttcaacgctgacaaaggcttcggcttcatcactcctgacgatggctctaaagatgtgttcgtacacttctctgctatccagaacgatggttacaaatctctggacgaaggtcagaaagtgtccttcaccatcgaaagcggcgctaaaggcccggcagctggtaacgtaaccagcctgtaatctctgcttaaaagcacagaatctaagatccctgccatttggcggggatttttttatttgttttcaggaaataaataatcgatcgcgtaataaaatctattattatttttgtgaagaataaatttgggtgcaatgagaatgcgcaacgccgtaagtaaggcgggaataatttcccgccgaagactcttactctttcaatt

SEQ ID NO: 18 - protein, 70 aa, Escherichia coli
MSGKMTGIVKWFNADKGFGFITPDDGSKDVFVHFSAIQNDGYKSLDEGQKVSFTIESGAKGPAAGNVTSL

SEQ ID NO: 19 - DNA, 601 bp, Bacillus subtilis
  gene (1)..(601): cspB
ttgaaaaataaatatggtattttccgaaaaaaatttcaataagcagttgttttttctgaagattactggtagagtaaaggtaattatttttgttcgaactatctttaagaagaaagttttgtaagagttttcgtcttgaaagtttgttaagagcaagaatagtgaatttaagcgttatgatcgctttaggaggaaatttcatgttagaaggtaaagtaaaatggttcaactctgaaaaaggtttcggattcatcgaagtagaaggtcaagacgatgtattcgttcatttctctgctattcaaggcgaaggcttcaaaactttagaagaaggccaagctgtttcttttgaaatcgttgaaggaaaccgcggaccacaagctgctaacgttactaaagaagcgtaagcataaattgatatgaaaaactgcaggtgcaaacctgcagtttttattttgacaaaaaagctgttttgcataagcaaaacagctttttttattagtttgcaatatcccgcttcatgaagattccgaaagcaagcaacaggaagatgataaagtagatcgccagcatgacgagagaaaacgtcatagtcatccctttt

SEQ ID NO: 20 - protein, 67 aa, Bacillus subtilis
MLEGKVKWFNSEKGFGFIEVEGQDDVFVHFSAIQGEGFKTLEEGQAVSFEIVEGNRGPQAANVTKEA

SEQ ID NO: 21 - DNA, 616 bp, Escherichia coli
  gene (1)..(616): cspB
aacattgcttcatatgttcaactatgcgttaatgattgcgtcggtttgaagaacagacgatatacgaagtagtttactaaagcagttctcatttcaggtgttattcacttattccttctttgagtctctccaattaagtacgaagtcgtttctgttatgcaaaccatttatgccgaaaggctcaagttaaggaatgtagaatgtcaaataaaatgactggtttagtaaaatggtttaacgctgataaaggtttcggctttatttctcctgttgatggtagtaaagatgtgtttgtgcatttttctgcgattcagaatgataattatcgaaccttatttgaaggtcaaaaggttaccttctctatagagagtggtgctaaaggtcctgcagcagcaaatgtcatcattactgattaaaattcatcgctcgtctgtatacgataacgaagaaggctgatgcctgagtagagatacggacagagtagtgaatattggatctctttaataaaaagtaaggaggtccaatacatgaaacaatggctagcatatttggcaaaatcttaatcaggaaaagtatgctaaccattgtggtgaagtgcaggtttgctgcatgaata

SEQ ID NO: 22 - protein, 71 aa, Escherichia coli
MSNKMTGLVKWFNADKGFGFISPVDGSKDVFVHFSAIQNDNYRTLFEGQKVTFSIESGAKGPAAANVIITD

SEQ ID NO: 23 - DNA, 1456 bp, Bacillus subtilis
  gene (1)..(1456): des
atttcgggtacatcacgaatatggattgataaacattaatcagcttgtaaatttttttacaagcttttttagcgcaatcggctatgcatgccgcacgagacatgacaaatgtcatataggaggcatgatgtgtgctactacaaaagacttctctcattagcgtatactgaaccgagacacacaatgagaggatacttactatgactgaacaaaccattgcacataaacaaaaacagctgacaaagcaagtcgctgcatttgctcagcctgaaacaaaaaacagcctgattcagcttttaaacacgtttatcccattcttcggcctatggtttcttgcttatctcagcctcgatgtctcctatcttcttacgttagcattaacggtgattgccgcaggttttctgacaagaatttttatcatcttccatgactgctgccatcaatcttttttcaaacaaaaacgctataaccacattctcggttttctgacaggtgtcctgactttattcccgtatcttcaatggcagcacagccattcgattcatcatgcaactagcagcaatctggataaacgcggaacaggagacatctggatgttaacagtaaacgaatataaagctgcatccagacgaacaaagcttgcatacagactttatagaaacccgtttatcatgtttattctcggaccgatttatgtttttctgatcacgaaccgttttaacaaaaaaggcgcaagacgcaaggaacgtgtaaacacataccttacgaatctggcaattgtcgcgttggctgccgcttgctgtctgatctttggctggcaatcgtttttactggtgcaaggcccgatatttctgatttcaggttcaatcggtgtttggctgttttatgtgcagcatacctttgaagattcttattttgaagcggatgaaaactggagctacgttcaggctgctgttgaaggcagctcattttataaactcccgaaactgcttcaatggctaacaggcaatattggttaccaccacgttcatcatttgagtccaaaggtgcctaactataagcttgaagttgctcatgaacatcacgaaccattaaaaaacgtaccgacaatcaccttaaaaacaagcctgcaatcacttgcgttccgtctatgggatgaagataacaaacagtttgtgtcatttcgggctataaaacatatacctgtaagccttccgcctgattcaccagaaaaacagaagctgcggaagaatgcctgatgataaaggaggtcttctaatatactagaaggcttcctttttattgttggagggtgcattctggctttcacaatgataaagttacaagtaagatgaaaagctgaaaatgaggtaagatcatgattaaaaatcattttacatttcaaaaactaaacgggattacgccctatatatggacgatatttttcatcctcccc

SEQ ID NO: 24 - protein, 352 aa, Bacillus subtilis
MTEQTIAHKQKQLTKQVAAFAQPETKNSLIQLLNTFIPFFGLWFLAYLSLDVSYLLTLALTVIAAGFLTRIFIIFHDCCHQSFFKQKRYNHILGFLTGVLTLFPYLQWQHSHSIHHATSSNLDKRGTGDIWMLTVNEYKAASRRTKLAYRLYRNPFIMFILGPIYVFLITNRFNKKGARRKERVNTYLTNLAIVALAAACCLIFGWQSFLLVQGPIFLISGSIGVWLFYVQHTFEDSYFEADENWSYVQAAVEGSSFYKLPKLLQWLTGNIGYHHVHHLSPKVPNYKLEVAHEHHEPLKNVPTITLKTSLQSLAFRLWDEDNKQFVSFRAIKHIPVSLPPDSPEKQKLRKNA

SEQ ID NO: 25 - DNA, 2233 bp, Bacillus subtilis
  gene (1)..(2233): dnaK
gaaggcgtcgaagccatcgaagctgtagggcaggaatttgatcctaatctgcaccaagctgttatgcaggctgaagacgaaaactacggctccaacattgttgttgaggaaatgcaaaaaggctataagctgaaggatcgcgtcattcgcccttccatggtcaaagtgaatcaataattacatagcaggaggttattcaagtgagtaaagttatcggaatcgacttaggaacaacaaactcatgtgtggcagtgcttgaaggcggcgagcctaaagttattgctaacgctgaaggaaaccgcacaacgccatcagttgttgcatttaaaaacggcgaacgtcaagtaggggaagtggctaaacgccaatctattacaaaccctaacacaattatgtctatcaaacgtcatatgggtactgattataaagttgaaattgaaggaaaggattacactccacaagaagtgtctgctatcatccttcaacaccttaaatcatacgctgaaagctatcttggcgaaacagtatcaaaagcagttatcacagttcctgcatactttaacgatgctgagcgtcaagcaacaaaagacgctggtaaaattgcaggtcttgaagtagaacgtatcatcaacgagccgactgcagcagcgcttgcatacggacttgataaaacagatgaagatcaaacgatcctagtatacgaccttggcggcggtacattcgacgtttccatccttgagcttggcgacggtgtattcgaagttcgttcaactgccggcgacaaccgtctgggtggggacgattttgaccaagttatcatcgatcatctggtgtctgaattcaaaaaagaaaacggcattgatttgtcaaaagacaaaatggcgcttcagcgtttgaaagacgcagctgaaaaagcgaaaaaagatctttccggcgtatcttctacgcaaatttctttaccgtttatcacagctggagaagcaggaccgcttcaccttgaacttacattaactcgcgctaaattcgaagagctttcttctcatttagtagagcgcacaatgggtcctgtccgtcaagcgcttcaagatgcaggactttctgcaagcgaaatcgacaaagtcatccttgtcggcggatcaactcgtatccctgccgtacaagaagcaatcaaaaaagaaactggaaaagaagcgcataaaggcgtaaacccggatgaagttgtagcgcttggtgctgcgattcagggcggcgttatcacaggtgacgtaaaagatgttgttcttcttgacgttacaccgctttctctcggtatcgaaacaatgggcggtgtgtttacaaaactgatcgaccgcaacacgacgatcccaacaagcaaatctcaagtgttctcaactgctgctgataaccaaacagctgttgatatccatgttcttcaaggtgagcgcccaatgtctgccgacaacaaaacactcggccgcttccagcttactgatatcccgccagcaccgcgcggcgtgcctcaaatcgaagtttctttcgatattgacaaaaacggtatcgtaaacgtaagagcaaaagacttaggcacaggaaaagaacaaaacattacaatcaaatcttcttcaggtctctcagatgaagagatcgaacgcatggtaaaagaagcggaagaaaatgctgacgctgatgcgaagaaaaaagaagaaatcgaagtccgcaacgaagcagatcagcttgttttccaaactgagaaaacattaaaagatcttgaaggcaaagtggacgaagaacaagtgaaaaaagccaacgatgccaaagacgctttaaaagcagcgattgagaaaaacgaatttgaagagatcaaagcgaaaaaagatgagcttcaaacaatcgttcaagagctttctatgaagctttatgaagaagctgctaaagcacagcaagctcaaggcggagcaaacgctgaaggcaaagcggatgacaacgttgtcgacgctgaatacgaagaagtaaacgacgaccaaaacaaaaaataagttctttttagtgtcagccccgcttccggagctgaccgaaaagaacacatttcataatctgattcaatgattagaaagtcaaagtcaggcatctcttggctttgactttttttcttgcccgggataaaaggaattgaaaaatcataattcaaaatgatacaatctaatttatgtgagaaattcgggagagtgaagcg

SEQ ID NO: 26 - protein, 611 aa, Bacillus subtilis
VSKVIGIDLGTTNSCVAVLEGGEPKVIANAEGNRTTPSVVAFKNGERQVGEVAKRQSITNPNTIMSIKRHMGTDYKVEIEGKDYTPQEVSAIILQHLKSYAESYLGETVSKAVITVPAYFNDAERQATKDAGKIAGLEVERIINEPTAAALAYGLDKTDEDQTILVYDLGGGTFDVSILELGDGVFEVRSTAGDNRLGGDDFDQVIIDHLVSEFKKENGIDLSKDKMALQRLKDAAEKAKKDLSGVSSTQISLPFITAGEAGPLHLELTLTRAKFEELSSHLVERTMGPVRQALQDAGLSASEIDKVILVGGSTRIPAVQEAIKKETGKEAHKGVNPDEVVALGAAIQGGVITGDVKDVVLLDVTPLSLGIETMGGVFTKLIDRNTTIPTSKSQVFSTAADNQTAVDIHVLQGERPMSADNKTLGRFQLTDIPPAPRGVPQIEVSFDIDKNGIVNVRAKDLGTGKEQNITIKSSSGLSDEEIERMVKEAEENADADAKKKEEIEVRNEADQLVFQTEKTLKDLEGKVDEEQVKKANDAKDALKAAIEKNEFEEIKAKKDELQTIVQELSMKLYEEAAKAQQAQGGANAEGKADDNVVDAEYEEVNDDQNKK

SEQ ID NO: 27 - DNA, 2317 bp, Escherichia coli
  gene (1)..(2317): dnaK
catatcgcgaaatttctgcgcaaaagcacaaaaaatttttgcatctcccccttgatgacgtggtttacgaccccatttagtagtcaaccgcagtgagtgagtctgcaaaaaaatgaaattgggcagttgaaaccagacgtttcgcccctattacagactcacaaccacatgatgaccgaatatatagtggagacgtttagatgggtaaaataattggtatcgacctgggtactaccaactcttgtgtagcgattatggatggcaccactcctcgcgtgctggagaacgccgaaggcgatcgcaccacgccttctatcattgcctatacccaggatggtgaaactctagttggtcagccggctaaacgtcaggcagtgacgaacccgcaaaacactctgtttgcgattaaacgcctgattggtcgccgcttccaggacgaagaagtacagcgtgatgtttccatcatgccgttcaaaattattgctgctgataacggcgacgcatgggtcgaagttaaaggccagaaaatggcaccgccgcagatttctgctgaagtgctgaaaaaaatgaagaaaaccgctgaagattacctgggtgaaccggtaactgaagctgttatcaccgtaccggcatactttaacgatgctcagcgtcaggcaaccaaagacgcaggccgtatcgctggtctggaagtaaaacgtatcatcaacgaaccgaccgcagctgcgctggcttacggtctggacaaaggcactggcaaccgtactatcgcggtttatgacctgggtggtggtactttcgatatttctattatcgaaatcgacgaagttgacggcgaaaaaaccttcgaagttctggcaaccaacggtgatacccacctggggggtgaagacttcgacagccgtctgatcaactatctggttgaagaattcaagaaagatcagggcattgacctgcgcaacgatccgctggcaatgcagcgcctgaaagaagcggcagaaaaagcgaaaatcgaactgtcttccgctcagcagaccgacgttaacctgccatacatcactgcagacgcgaccggtccgaaacacatgaacatcaaagtgactcgtgcgaaactggaaagcctggttgaagatctggtaaaccgttccattgagccgctgaaagttgcactgcaggacgctggcctgtccgtatctgatatcgacgacgttatcctcgttggtggtcagactcgtatgccaatggttcagaagaaagttgctgagttctttggtaaagagccgcgtaaagacgttaacccggacgaagctgtagcaatcggtgctgctgttcagggtggtgttctgactggtgacgtaaaagacgtactgctgctggacgttaccccgctgtctctgggtatcgaaaccatgggcggtgtgatgacgacgctgatcgcgaaaaacaccactatcccgaccaagcacagccaggtgttctctaccgctgaagacaaccagtctgcggtaaccatccatgtgctgcagggtgaacgtaaacgtgcggctgataacaaatctctgggtcagttcaacctagatggtatcaacccggcaccgcgcggcatgccgcagatcgaagttaccttcgatatcgatgctgacggtatcctgcacgtttccgcgaaagataaaaacagcggtaaagagcagaagatcaccatcaaggcttcttctggtctgaacgaagatgaaatccagaaaatggtacgcgacgcagaagctaacgccgaagctgaccgtaagtttgaagagctggtacagactcgcaaccagggcgaccatctgctgcacagcacccgtaagcaggttgaagaagcaggcgacaaactgccggctgacgacaaaactgctatcgagtctgcgctgactgcactggaaactgctctgaaaggtgaagacaaagccgctatcgaagcgaaaatgcaggaactggcacaggtttcccagaaactgatggaaatcgcccagcagcaacatgcccagcagcagactgccggtgctgatgcttctgcaaacaacgcgaaagatgacgatgttgtcgacgctgaatttgaagaagtcaaagacaaaaaataatcgccctataaacgggtaattatactgacacgggcgaaggggaatttcctctccgcccgtgcattcatctaggggcaatttaaaaaagatggctaagcaagattattacgagattttaggcgtttccaaaacagcggaagagcgtgaaatcagaaaggcctacaaacgcctggccatgaaataccacccggaccgtaacc

SEQ ID NO: 28 - protein, 638 aa, Escherichia coli
MGKIIGIDLGTTNSCVAIMDGTTPRVLENAEGDRTTPSIIAYTQDGETLVGQPAKRQAVTNPQNTLFAIKRLIGRRFQDEEVQRDVSIMPFKIIAADNGDAWVEVKGQKMAPPQISAEVLKKMKKTAEDYLGEPVTEAVITVPAYFNDAQRQATKDAGRIAGLEVKRIINEPTAAALAYGLDKGTGNRTIAVYDLGGGTFDISIIEIDEVDGEKTFEVLATNGDTHLGGEDFDSRLINYLVEEFKKDQGIDLRNDPLAMQRLKEAAEKAKIELSSAQQTDVNLPYITADATGPKHMNIKVTRAKLESLVEDLVNRSIEPLKVALQDAGLSVSDIDDVILVGGQTRMPMVQKKVAEFFGKEPRKDVNPDEAVAIGAAVQGGVLTGDVKDVLLLDVTPLSLGIETMGGVMTTLIAKNTTIPTKHSQVFSTAEDNQSAVTIHVLQGERKRAADNKSLGQFNLDGINPAPRGMPQIEVTFDIDADGILHVSAKDKNSGKEQKITIKASSGLNEDEIQKMVRDAEANAEADRKFEELVQTRNQGDHLLHSTRKQVEEAGDKLPADDKTAIESALTALETALKGEDKAAIEAKMQELAQVSQKLMEIAQQQHAQQQTAGADASANNAKDDDVVDAEFEEVKDKK

SEQ ID NO: 29 - DNA, 1690 bp, Bacillus subtilis
  gene (1)..(1690): eno
ggcgaaccgcacactgcgcatacaacaaacccagtccctgtgattgtaacgaaagaaggcattacgctgcgtgaaggcggaatcctaggcgaccttgcaccaacgttattagaccttcttggtgttgaaaaaccgaaagaaatgacaggaacatctttaattcaaaaataatttgaaaatcaaaaggagagacaaaactcatgccatacattgttgatgtttatgcacgcgaagtattagactcccgcggcaacccaacagttgaagttgaagtatatacagaaacaggagctttcggccgcgcattagtgccaagcggagcttctacaggtgaatacgaagcggttgagcttcgtgacggcgacaaagaccgttaccttggaaaaggcgtgttaacagctgttaacaacgtaaacgaaatcattgctccagagcttcttggctttgatgtaactgaacaaaacgcaatcgatcagcttttaatcgagcttgacggtactgaaaacaaaggcaaacttggtgcgaacgcaatccttggcgtatctatggcttgtgcgcgtgctgctgctgatttcttacagattcctctttaccaataccttggaggattcaactcaaaaacgcttcctgtaccgatgatgaacatcgtaaacggcggagagcatgctgataacaacgtggatattcaagaattcatgatcatgcctgtaggtgctcctaacttccgtgaagcacttcgcatgggcgctcaaatcttccacagcctgaaatcagtattaagcgcaaaaggcttaaacactgctgtaggtgacgaaggcggattcgctccaaaccttggttctaacgaagaagcgcttcaaacaatcgttgaagcaatcgaaaaagccggcttcaaacctggcgaagaagttaaacttgctatggatgctgcatcttctgagttctacaacaaagaagacggcaaataccatctgtctggcgaaggcgttgtgaaaacatctgctgaaatggttgactggtatgaagagcttgtttctaaatacccaatcatctcgatcgaagacggacttgacgaaaacgactgggaaggccacaagcttcttactgagcgtcttggcaaaaaagttcagcttgttggtgacgacctcttcgttacaaacacgaaaaaactttctgaaggtattaaaaacggcgtaggcaactctatcctgatcaaagtaaaccaaatcggtacattgactgaaacattcgatgcgatcgaaatggcgaaacgcgcaggctacacagctgttatctctcaccgttctggtgaaactgaagacagcacaatcgctgacatcgctgtggcaacaaacgcaggacaaatcaaaacaggtgctccgtctcgtacggaccgtgttgcgaaatacaaccagcttcttcgcatcgaagatcagttggctgaaactgctcaataccacggtatcaactctttctacaacttaaacaagtaataaaaagaccgccgggattttctctcggcggcttttttatgctttcagagatccaaataaagccttaccatatcgcggcattgaatcccgttttcaaaaatgtcctcatcataatgtctgagaaagaaatcgtggtcgatcgcctgaatgcggaatccgcatttttgatagagggaaagctgatgaatgctcgaatt

SEQ ID NO: 30 - protein, 430 aa, Bacillus subtilis
MPYIVDVYAREVLDSRGNPTVEVEVYTETGAFGRALVPSGASTGEYEAVELRDGDKDRYLGKGVLTAVNNVNEIIAPELLGFDVTEQNAIDQLLIELDGTENKGKLGANAILGVSMACARAAADFLQIPLYQYLGGFNSKTLPVPMMNIVNGGEHADNNVDIQEFMIMPVGAPNFREALRMGAQIFHSLKSVLSAKGLNTAVGDEGGFAPNLGSNEEALQTIVEAIEKAGFKPGEEVKLAMDAASSEFYNKEDGKYHLSGEGVVKTSAEMVDWYEELVSKYPIISIEDGLDENDWEGHKLLTERLGKKVQLVGDDLFVTNTKKLSEGIKNGVGNSILIKVNQIGTLTETFDAIEMAKRAGYTAVISHRSGETEDSTIADIAVATNAGQIKTGAPSRTDRVAKYNQLLRIEDQLAETAQYHGINSFYNLNK

SEQ ID NO: 31 - DNA, 805 bp, Bacillus subtilis
  gene (1)..(805): glnR
gcgtatgttcagggaggattaacctattcgcatgtgaagaatgccatatgcagtgcagtggattcattgatgcaaaagcaattaatttaaattttttaaaaatttctcctggatttgatgttaagaatccttacatcgtattgacacataatataacatcacctataatgaaactaagttaagaaaaggaggaaattgagatgagtgataatattcgccgctcaatgcctttatttccaataggaattgtcatgcagttaactgagttatcagcaagacaaattcgatattatgaggaaaatggactgatatttccagccagaagtgaaggaaatagacgattattttcatttcatgatgtagataaactgttagaaatcaagcacctgatagaacaaggtgtaaacatggcaggaattaaacagattctggcgaaagccgaagccgagccagaacaaaaacaaaacgagaagacgaaaaaaccaatgaaacatgatctgtccgatgacgaactgagacagctcctgaaaaacgagctcatgcaagccggccgttttcaaagagggaatacattccgtcaaggcgacatgtcccgcttctttcattaatccgttagcaactgttttgctatatacatttttaccttttaagaggaggaattttaccaaatggcaaagtacactagagaagatatcgaaaaattagtaaaagaagaaaacgtgaagtatatccgccttcaatttactgacattcttggaacaatcaagaatgttgagattcctgtaagccagcttggaaaagcgct

SEQ ID NO: 32 - protein, 135 aa, Bacillus subtilis
MSDNIRRSMPLFPIGIVMQLTELSARQIRYYEENGLIFPARSEGNRRLFSFHDVDKLLEIKHLIEQGVNMAGIKQILAKAEAEPEQKQNEKTKKPMKHDLSDDELRQLLKNELMQAGRFQRGNTFRQGDMSRFFH

SEQ ID NO: 33 - DNA, 2032 bp, Bacillus subtilis
  gene (1)..(2032): groEL
aggtcgcgtgttagaaagcggagagcgcgttgctttagaagtaaaagagggcgaccgcattatcttctcaaaatacgcaggtactgaagtgaaatacgaaggtactgaatacttaatcttacgtgaaagcgacattttagctgttatcggctaattcttaaataaacaatacttaaaacatttgaggaggtcttgtaaacatggcaaaagaaattaagtttagtgaagaagctcgccgcgcaatgcttcgcggtgtcgatgcacttgctgatgctgttaaagtaactttaggaccaaaaggacgcaacgtggttctagagaaaaaattcggttctccgttaatcacaaatgacggtgtaacaatcgctaaagaaatcgagctagaagacgcgtttgaaaacatgggtgctaagcttgttgctgaagtagccagcaaaacaaacgacgttgccggtgacggtacaacaactgcaacagttcttgcgcaagcaatgatccgtgaaggccttaaaaacgtaacagcaggcgctaaccctgtaggcgtgcgtaaagggatggaacaagctgtagcggttgcgatcgaaaacttaaaagaaatttctaagccaatcgaaggcaaagagtctatcgctcaggttgctgcgatctctgctgctgatgaggaagtcggaagccttatcgctgaagcaatggagcgcgtaggaaacgacggcgttatcacaatcgaagagtctaaaggcttcacaactgagcttgaagttgttgaaggtatgcaattcgaccgcggatatgcgtctccttacatggtaactgactctgataagatggaagcggttcttgacaatccttacatcttaatcacagacaaaaaaatcacaaacattcaagaaatccttcctgtgcttgagcaggttgttcagcaaggcaaaccattgcttctgatcgctgaggatgttgaaggcgaagcacttgctacacttgttgtgaacaaacttcgcggcacattcaacgcagtggctgttaaagctcctggtttcggtgatcgccgtaaagcaatgcttgaagacatcgctgtccttactggcggagaagtcatcacagaagatcttggccttgacctgaaatctactcaaatcgctcaattgggacgcgcttctaaagttgtcgttactaaagaaaacacaacaatcgttgaaggcgctggcgaaacagacaaaatttctgcccgtgtgactcaaatccgcgctcaagtggaagaaacaacttctgaattcgacagagaaaaattacaagagcgtcttgctaaacttgctggcggcgtagctgtcatcaaagtcggcgctgcgactgaaactgagctgaaagagcgtaaacttcgcatcgaagacgccttgaactcaactcgcgcagctgttgaagaaggcatcgtatccggtggtggtacagcgcttgtaaacgtatataacaaagtcgctgcagttgaagctgaaggcgatgctcaaacaggtatcaacatcgtgcttcgcgcgcttgaagagccaatccgtcaaatcgcacacaacgctggccttgaaggatctgtcatcgttgagcgcctcaaaaacgaagaaatcggcgtaggcttcaacgctgcaactggcgaatgggtaaacatgatcgaaaaaggtatcgttgacccaacaaaagttacacgctcagctcttcaaaacgctgcgtctgtagctgcaatgttcttaacaacagaagctgttgtcgctgacaagccagaagaaaacggtggcggcgcaggaatgcctgatatgggcggcatgggcggtatgggtggaatgatgtaataaagggatattaacccttatacatcaatgttttaaccgtcttaaaaaactagacaaagcgtgaataaaaaaagagaaggtctttcatcagtttactaaactgttgggagaccttttctccatattagcggtcatatgagcataaatgtccatcgtcgtatttatgtccgaatgaccaaattgttgctggatttctt

SEQ ID NO: 34 - protein, 544 aa, Bacillus subtilis
MAKEIKFSEEARRAMLRGVDALADAVKVTLGPKGRNVVLEKKFGSPLITNDGVTIAKEIELEDAFENMGAKLVAEVASKTNDVAGDGTTTATVLAQAMIREGLKNVTAGANPVGVRKGMEQAVAVAIENLKEISKPIEGKESIAQVAAISAADEEVGSLIAEAMERVGNDGVITIEESKGFTTELEVVEGMQFDRGYASPYMVTDSDKMEAVLDNPYILITDKKITNIQEILPVLEQVVQQGKPLLLIAEDVEGEALATLVVNKLRGTFNAVAVKAPGFGDRRKAMLEDIAVLTGGEVITEDLGLDLKSTQIAQLGRASKVVVTKENTTIVEGAGETDKISARVTQIRAQVEETTSEFDREKLQERLAKLAGGVAVIKVGAATETELKERKLRIEDALNSTRAAVEEGIVSGGGTALVNVYNKVAAVEAEGDAQTGINIVLRALEEPIRQIAHNAGLEGSVIVERLKNEEIGVGFNAATGEWVNMIEKGIVDPTKVTRSALQNAASVAAMFLTTEAVVADKPEENGGGAGMPDMGGMGGMGGMM

SEQ ID NO: 35 - DNA, 2047 bp, Escherichia coli
  gene (1)..(2047): groL
ccgtatccttgaaaatggcgaagtgaagccgctggatgtgaaagttggcgacatcgttattttcaacgatggctacggtgtgaaatctgagaagatcgacaatgaagaagtgttgatcatgtccgaaagcgacattctggcaattgttgaagcgtaatccgcgcacgacactgaacatacgaatttaaggaataaagataatggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccatgctctgactctaaagcgattgctcaggttggtaccatctccgctaactccgacgaaaccgtaggtaaactgatcgctgaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgccaaagcaggcaaaccgctgctgatcatcgctgaagatgtagaaggcgaagcgctggcaactctggttgttaacaccatgcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtgggtgctgctaccgaagttgaaatgaaagagaaaaaagcacgcgttgaagatgccctgcacgcgacccgtgctgcggtagaagaaggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgaatgcatggttaccgacctgccgaaaaacgatgcagctgacttaggcgctgctggcggtatgggcggcatgggtggcatgggcggcatgatgtaattgccctgcacctcgcagaaataaacaaacccccgggcagaaatgtctgggggtttttcttttggtcatctttctagtataagattcagacacggacgacgcgagtggcgtccagctcattgattatggggaataacatgcacgtaaaatacttagcagggattgtcggtgccgcgctactgatggcgggttgtagctcc

SEQ ID NO: 36 - protein, 548 aa, Escherichia coli
MAAKDVKFGNDARVKMLRGVNVLADAVKVTLGPKGRNVVLDKSFGAPTITKDGVSVAREIELEDKFENMGAQMVKEVASKANDAAGDGTTTATVLAQAIITEGLKAVAAGMNPMDLKRGIDKAVTAAVEELKALSVPCSDSKAIAQVGTISANSDETVGKLIAEAMDKVGKEGVITVEDGTGLQDELDVVEGMQFDRGYLSPYFINKPETGAVELESPFILLADKKISNIREMLPVLEAVAKAGKPLLIIAEDVEGEALATLVVNTMRGIVKVAAVKAPGFGDRRKAMLQDIATLTGGTVISEEIGMELEKATLEDLGQAKRVVINKDTTTIIDGVGEEAAIQGRVAQIRQQIEEATSDYDREKLQERVAKLAGGVAVIKVGAATEVEMKEKKARVEDALHATRAAVEEGVVAGGGVALIRVASKLADLRGQNEDQNVGIKVALRAMEAPLRQIVLNCGEEPSVVANTVKGGDGNYGYNAATEEYGNMIDMGILDPTKVTRSALQYAASVAGLMITTECMVTDLPKNDAADLGAAGGMGGMGGMGGMM

SEQ ID NO: 37 - DNA, 769 bp, Bacillus subtilis
  gene (1)..(769): gsiB
acaggcatgatgctgtacaaaaagaggcgcattcttgaacgactgaaagaaacagaacgagagatggaatagcagaaagcagacggacaccgcgatccgcctgctttttttagtggaaacatacccaatgtgttttgtttgtttaaaagaattgtgagcgggaatacaacaaccaacaccaattaaaggaggaattcaaaatggcagacaataacaaaatgagcagagaagaagcaggtagaaaaggcggagaaacaacaagcaagaaccatgacaaagaattctatcaagagattggtcaaaaaggcggagaagccactagcaaaaaccatgacaaagaattctatcaggaaatcggcgaaaaaggcggagaagccactagcaagaaccatgacaaagagttctatcaagaaatcggtgaaaaaggcggagaagccactagcgaaaatcatgacaaagaattctatcaagaaatcggccgaaaaggcggagaggcaacaagcaaaaatcatgataaagaattctaccaagaaattggctcaaaaggcggaaacgcccgcaacaacgactaatcaaaagcataaaggcagacaacctgtaaataaaaaagcgatccggcacatcagctggatcgcttttttttggaatcttctcaatttatgccgctattgagatacaataagtgaaaaaggggttttcgctaatgtccaagatgcaaaacttgctcgccatcgcaaatgaagtcaaagaaaagcacgccaatttttca

SEQ ID NO: 38 - protein, 123 aa, Bacillus subtilis
MADNNKMSREEAGRKGGETTSKNHDKEFYQEIGQKGGEATSKNHDKEFYQEIGEKGGEATSKNHDKEFYQEIGEKGGEATSENHDKEFYQEIGRKGGEATSKNHDKEFYQEIGSKGGNARNND

SEQ ID NO: 39 - DNA, 814 bp, Escherichia coli
  gene (1)..(814): ibpA
aacgtttcgtgctgccccctcagtctatgcaatagaccataaactgcaaaaaaaagtccgctgataaggcttgaaaagttcatttccagacccatttttacatcgtagccgatgaggacgcgcctgatgggtgttctggctacctgacctgtccattgtggaaggtcttacattctcgctgatttcaggagctattgattatgcgtaactttgatttatccccgctttaccgttctgctattggatttgaccgtttgtttaaccacttagaaaacaaccagagccagagtaatggcggctaccctccgtataacgttgaactggtagacgaaaaccattaccgcattgctatcgctgtggctggttttgctgagagcgaactggaaattaccgcccaggataatctgctggtggtgaaaggtgctcacgccgacgaacaaaaagagcgcacctatctgtaccagggcatcgctgaacgcaactttgaacgcaaattccagttagctgagaacattcatgttcgtggtgctaacctggtaaatggtttgctgtatatcgatctcgaacgcgtgattccggaagcgaaaaaaccgcgccgtatcgaaatcaactaattccctaaggccgcctggcgcggcctgacatctccatgctcgccgtcagggagcatatgcgaatcttcggatttgcaggtacttactcgcttcttagaaggagaaatgactatgcgtaacttcgatttatccccactgatgcgtcaatggatcggttttgacaaactggccaacgcactgcaaaacgccggtgaaagcca

SEQ ID NO: 40 - protein, 137 aa, Escherichia coli
MRNFDLSPLYRSAIGFDRLFNHLENNQSQSNGGYPPYNVELVDENHYRIAIAVAGFAESELEITAQDNLLVVKGAHADEQKERTYLYQGIAERNFERKFQLAENIHVRGANLVNGLLYIDLERVIPEAKKPRRIEIN

SEQ ID NO: 41 - DNA, 829 bp, Escherichia coli
  gene (1)..(829): ibpB
gtgctaacctggtaaatggtttgctgtatatcgatctcgaacgcgtgattccggaagcgaaaaaaccgcgccgtatcgaaatcaactaattccctaaggccgcctggcgcggcctgacatctccatgctcgccgtcagggagcatatgcgaatcttcggatttgcaggtacttactcgcttcttagaaggagaaatgactatgcgtaacttcgatttatccccactgatgcgtcaatggatcggttttgacaaactggccaacgcactgcaaaacgccggtgaaagccagagcttcccgccgtacaacattgagaaaagcgacgataaccactaccgcattacccttgcgctggcaggtttccgtcaggaagatttagagattcaactggaaggtacgcgcctgagcgtaaaaggcacgccggagcagccaaaagaagagaaaaaatggctgcatcaagggcttatgaatcagccatttagcctgagctttacgctggctgaaaatatggaagtctctggcgcaaccttcgtaaacggtttactgcatattgatttaattcgtaatgagcctgaacccatcgcagcgcagcgtatcgctatcagcgaacgtcccgcgttaaatagctaactagctattctctttgccccgccattctggtggggctttttttgtcttacttactcaacaatgcgttaaatttcgactgtttaagatatttcggcacgttctttcccgtgataatgtgcgctataaggcaaatctgcttcaccgattcagccataatccgtagcaattaaaatgttatctcaagagaaatggacgatgag

SEQ ID NO: 42 - protein, 142 aa, Escherichia coli
MRNFDLSPLMRQWIGFDKLANALQNAGESQSFPPYNIEKSDDNHYRITLALAGFRQEDLEIQLEGTRLSVKGTPEQPKEEKKWLHQGLMNQPFSLSFTLAENMEVSGATFVNGLLHIDLIRNEPEPIAAQRIAISERPALNS

SEQ ID NO: 43 - DNA, 1849 bp, Bacillus subtilis
  gene (1)..(1849): katA
tgtttcgcaaatagagaaggaaaaattatttctccaaaaattattaagtgtcataaaaagctgttacaacaaggtttttcaacaatttcgatactatatatcgattaatagagataactattttataataattataaaataatattgactttttacttagagatgatattatgttcttataaattccaagaggtgataacatgagttcaaataaactgacaactagctggggcgctccggttggagataatcaaaactcaatgactgccggttctcgcggaccaactttaattcaagatgtacatttactcgaaaaattggcccatttcaaccgagaacgtgttcctgaacgtgttgttcacgccaaaggagcaggcgcacacggatattttgaagtgacaaacgacgtaacaaaatacacgaaagccgctttcctttctgaagtcggcaaacgcacaccgttgttcatccgtttctcaacagttgccggtgaacttggctctgctgacacagttcgcgacccgcgcggatttgctgttaaattttatactgaagaaggaaactacgacatcgtcggcaacaatacgcctgtattctttatccgcgatgcgattaagttccctgatttcatccatacacaaaaaagagatccaaaaacacacctgaaaaaccctacggctgtatgggatttctggtcactttcaccagagtcactgcaccaagtgacaatcctgatgtctgaccgcggaattcctgcgacacttcgccacatgcacggcttcggaagccatacattcaaatggacaaatgccgaaggcgaaggcgtatggattaaatatcactttaaaacagaacaaggcgtgaaaaaccttgatgtcaatacggcagcaaaaattgccggtgaaaaccctgattaccatacagaagaccttttcaacgcaatcgaaaacggtgattatcctgcatggaaactatatgtgcaaatcatgcctttagaagatgcaaatacgtaccgtttcgatccgtttgatgtcacaaaagtttggtctcaaaaagactacccgttaatcgaggtcggacgcatggttctagacagaaatccggaaaactactttgcagaggtagaacaagcgacattttcacctggaaccctcgtgcctggtattgatgtttcaccggataaaatgcttcaaggtcgactttttgcttatcatgatgcacaccgctaccgtgtcggtgcaaaccatcaagcgctgccaatcaaccgcgcacgcaacaaagtaaacaattatcagcgtgatgggcaaatgcgttttggtgataacggcggcggatctgtgtattacgagcctaacagcttcggcggtccaaaagagtcacctgaggataagcaagcagcatatccggtacaaggtatcgctgacagcgtaagctacgatcactacgatcactacactcaagccggcgatctgtatcgtttaatgagtgaagatgaacgtacccgccttgttgaaaatatcgttaatgccatgaagccggtagaaaaagaagaaatcaagctgcgccaaatcgagcacttctacaaagcggatcctgaatacggaaaacgcgtggcagaaggccttggattgccgattaaaaaagattcttaatggagaaatgcaaaaacccgttgtagtcagcgggtttttttatatttagttacacattgcgtatgcttctcatcaatcttcatataaatttcttccatgagttccgcgctgtttataatctctcgtttgtttttttcaatcagcagctcaagcggcaaaatctgattcccgtaggtttttcgcttcctgaatcgttt

SEQ ID NO: 44 - protein, 483 aa, Bacillus subtilis
MSSNKLTTSWGAPVGDNQNSMTAGSRGPTLIQDVHLLEKLAHFNRERVPERVVHAKGAGAHGYFEVTNDVTKYTKAAFLSEVGKRTPLFIRFSTVAGELGSADTVRDPRGFAVKFYTEEGNYDIVGNNTPVFFIRDAIKFPDFIHTQKRDPKTHLKNPTAVWDFWSLSPESLHQVTILMSDRGIPATLRHMHGFGSHTFKWTNAEGEGVWIKYHFKTEQGVKNLDVNTAAKIAGENPDYHTEDLFNAIENGDYPAWKLYVQIMPLEDANTYRFDPFDVTKVWSQKDYPLIEVGRMVLDRNPENYFAEVEQATFSPGTLVPGIDVSPDKMLQGRLFAYHDAHRYRVGANHQALPINRARNKVNNYQRDGQMRFGDNGGGSVYYEPNSFGGPKESPEDKQAAYPVQGIADSVSYDHYDHYTQAGDLYRLMSEDERTRLVENIVNAMKPVEKEEIKLRQIEHFYKADPEYGKRVAEGLGLPIKKDS

SEQ ID NO: 45 - DNA, 2458 bp, Bacillus subtilis
  gene (1)..(2458): katE
gatatgacctcccctattcaacagcttttttggtaagcgcttccatcctttctttctgtgaagaaggcttatttcaaatgtgtatgaccgcatatttatatgtagaccaaaaatcctaatgagcggattagcagtttatatgaagaacgccacgggtaaatgtgctgtagaatgtttcgttttaaaaagggggagcagacatgagtgatgaccaaaacaaacgtgtaaatgaacactcaaaggacgagcagcttgagcagtacaggaccgataacagcgggaagaaaatgacgacaaaccaaggcttgcgagtgtcagaggatgagcactcgttaaaagccggggttcgcggcccgacgctgatggaggattttcatttccgggagaaaatgacgcactttgaccatgagaggattccggagcgcgttgtgcatgcgcgcggatttggcgtgcatggcttttttcaggtttacgaaccaatgacggaatacacacgggccaaatttcttcaggacccttcggtgaagacaccggtatttgtccgtttttctacggttgcgggttctaaaggctccgcagacacggtgcgggatgccagaggctttgcgacaaaattttatactgaagaaggaaactatgaccttgtgggaaataacattccggtgtttttcattcaggacgcaatcaaatttccggatttggttcacgcgtttaagcctgagccacacaatgaaatgcctcaggctgcgactgctcacgatacattttgggattttgtcgccaataaccctgagtctgcccacatggtgatgtggacgatgtctgacagaggcatcccgcgcagctacagaatgatggaaggcttcggcgttcacacgttccgttttgtcaatgaacaaggaaaggcacgtttcgtgaaatttcactggaagccggtgctcggcgtgcattctctcgtatgggatgaagcgcaaaaaattgccgggaaagatccagatttccaccgccgtgacctgtgggaaacaattgaaaacggcggcaaggttgaatacgagctcggcgtgcagatgattgatgaggaagatgaattcaagtttgattttgatattcttgacccaactaagctgtggccggaagagcttgtgccggtgaagatcattggaaaaatgacgctgaaccgcaatcaggataatgtgtttgcggaaacagaacaagtcgcattccacccgggaaatgtcgtgccgggcatcgattttaccaatgacccgctattgcaggggcggcttttcccttatacggatacacagctcatccgtcttggcggcccgaacttccatgagattccgattaaccggccggtctgtccattccataacaaccaatacgacggctatcaccggatgacgatcaataagggacctgtcgcttatcataaaaactcactgcaaaacaatgatccgtcgcctgcgacagctgaagaaggcggctacgtccactatcaggaaaaagtcgagggcaaaaaaatccgccagcgaagcgacagcttcaacgattattactcacaggcgaagcttttctggaacagcatgtctcctgtggagaaacagcacatcatttctgctttctgcttcgaggtcgggaaggttaaaagcaaagacgtacagcggcaggtcgttgatgtcttcagcaatgtcgatgcagatttagcagaagaaatcgccaaaggagtaggcgtggcggcgcccgcaaagcgtaaagcatctaaagaaaatcttacttcgcctgcgttaagccaagcccgtacagtgaaaaccgcctcaacaaggaaagtcgctgtactcgccggaaacggatttcatgaaaaagagctgcaaacagtactggaagccttaaagcaagaaggaatcacagtggatatcatcagtcaaaatctggggtatatgacaagcggcagcggacagcagcttgaggccagcggcacgttcctgacggttgattctgttctgtatgatgccgtctatgccgcaggaggactggagttaaaagacaacaaacaagcgatggcgtttatcagagaagcttacaatcactacaaggcgatcggtgcggcaaatgaaggaattgaccttcttcaatcctctgtcgggacaacagaggggctaggcatcgtaacggctaaagatgaacctgactacaccgctttcagcaaggcatttatcgatgcggttgctgctcatcgccattgggatagacgaatttgatcggctcctgtatctttaaggaagatacttttcataaggagcatgacaaatgaaccgaaaacagctcactgaagcttacgaacgcgatgaaaatatgatgattttggtgtttgcccagtggtgcgtgaaccatgatcttgatccgatggagctgtacgccaaagcgtatccgcagcaaaagctgaatgaaagcttga

SEQ ID NO: 46 - protein, 686 aa, Bacillus subtilis
MSDDQNKRVNEHSKDEQLEQYRTDNSGKKMTTNQGLRVSEDEHSLKAGVRGPTLMEDFHFREKMTHFDHERIPERVVHARGFGVHGFFQVYEPMTEYTRAKFLQDPSVKTPVFVRFSTVAGSKGSADTVRDARGFATKFYTEEGNYDLVGNNIPVFFIQDAIKFPDLVHAFKPEPHNEMPQAATAHDTFWDFVANNPESAHMVMWTMSDRGIPRSYRMMEGFGVHTFRFVNEQGKARFVKFHWKPVLGVHSLVWDEAQKIAGKDPDFHRRDLWETIENGGKVEYELGVQMIDEEDEFKFDFDILDPTKLWPEELVPVKIIGKMTLNRNQDNVFAETEQVAFHPGNVVPGIDFTNDPLLQGRLFPYTDTQLIRLGGPNFHEIPINRPVCPFHNNQYDGYHRMTINKGPVAYHKNSLQNNDPSPATAEEGGYVHYQEKVEGKKIRQRSDSFNDYYSQAKLFWNSMSPVEKQHIISAFCFEVGKVKSKDVQRQVVDVFSNVDADLAEEIAKGVGVAAPAKRKASKENLTSPALSQARTVKTASTRKVAVLAGNGFHEKELQTVLEALKQEGITVDIISQNLGYMTSGSGQQLEASGTFLTVDSVLYDAVYAAGGLELKDNKQAMAFIREAYNHYKAIGAANEGIDLLQSSVGTTEGLGIVTAKDEPDYTAFSKAFIDAVAAHRHWDRRI

SEQ ID NO: 47 - DNA, 2662 bp, Escherichia coli
  gene (1)..(2662): katE
tggcttcactaaacgcatattaaaaatcagaaaaactgtagtttagccgatttagcccctgtacgtcccgctttgcgtgtatttcataacaccgtttccagaatagtctccgaagcgggatctggctggtggtctatagttagagagttttttgaccaaaacagcggccctttcagtaataaattaaggagacgagttcaatgtcgcaacataacgaaaagaacccacatcagcaccagtcaccactacacgattccagcgaagcgaaaccggggatggactcactggcacctgaggacggctctcatcgtccagcggctgaaccaacaccgccaggtgcacaacctaccgccccagggagcctgaaagcccctgatacgcgtaacgaaaaacttaattctctggaagacgtacgcaaaggcagtgaaaattatgcgctgaccactaatcagggcgtgcgcatcgccgacgatcaaaactcactgcgtgccggtagccgtggtccaacgctgctggaagattttattctgcgcgagaaaatcacccactttgaccatgagcgcattccggaacgtattgttcatgcacgcggatcagccgctcacggttatttccagccatataaaagcttaagcgatattaccaaagcggatttcctctcagatccgaacaaaatcaccccagtatttgtacgtttctctaccgttcagggtggtgctggctctgctgataccgtgcgtgatatccgtggctttgccaccaagttctataccgaagagggtatttttgacttcgttggcaataacacgccaatcttctttatccaggatgcgcataaattccccgattttgttcatgcggtaaaaccagaaccgcactgggcaattccacaagggcaaagtgcccacgatactttctgggattatgtttctctgcaacctgaaactctgcacaacgtgatgtgggcgatgtcggatcgcggcatcccccgcagttaccgcaccatggaaggcttcggtattcacaccttccgcctgattaatgccgaagggaaggcaacgtttgtacgtttccactggaaaccactggcaggtaaagcctcactcgtttgggatgaagcacaaaaactcaccggacgtgacccggacttccaccgccgcgagttgtgggaagccattgaagcaggcgattttccggaatacgaactgggcttccagttgattcctgaagaagatgaattcaagttcgacttcgatcttctcgatccaaccaaacttatcccggaagaactggtgcccgttcagcgtgtcggcaaaatggtgctcaatcgcaacccggataacttctttgctgaaaacgaacaggcggctttccatcctgggcatatcgtgccgggactggacttcaccaacgatccgctgttgcagggacgtttgttctcctataccgatacacaaatcagtcgtcttggtgggccgaatttccatgagattccgattaaccgtccgacctgcccttaccataatttccagcgtgacggcatgcatcgcatggggatcgacactaacccggcgaattacgaaccgaactcgattaacgataactggccgcgcgaaacaccgccggggccgaaacgcggcggttttgaatcataccaggagcgcgtggaaggcaataaagttcgcgagcgcagcccatcgtttggcgaatattattcccatccgcgtctgttctggctaagtcagacgccatttgagcagcgccatattgtcgatggtttcagttttgagttaagcaaagtcgttcgtccgtatattcgtgagcgcgttgttgaccagctggcgcatattgatctcactctggcccaggcggtggcgaaaaatctcggtatcgaactgactgacgaccagctgaatatcaccccacctccggacgtcaacggtctgaaaaaggatccatccttaagtttgtacgccattcctgacggtgatgtgaaaggtcgcgtggtagcgattttacttaatgatgaagtgagatcggcagaccttctggccattctcaaggcgctgaaggccaaaggcgttcatgccaaactgctctactcccgaatgggtgaagtgactgcggatgacggtacggtgttgcctatagccgctacctttgccggtgcaccttcgctgacggtcgatgcggtcattgtcccttgcggcaatatcgcggatatcgctgacaacggcgatgccaactactacctgatggaagcctacaaacaccttaaaccgattgcgctggcgggtgacgcgcgcaagtttaaagcaacaatcaagatcgctgaccagggtgaagaagggattgtggaagctgacagcgctgacggtagttttatggatgaactgctaacgctgatggcagcacaccgcgtgtggtcacgcattcctaagattgacaaaattcctgcctgatgggaggcggcgcaattgcgccgcctcaaatgatttacatagtgcgctttgtttatgccggatgcggcgtgaacgccttatccggcctacaaaactgtgcaaattcaatatattgcaggaaacacgtaggcctgataagcgaagcgcatcaggcagttttgcgtttgtcagcagtctcaagcggcgcagttacgccgcct

SEQ ID NO: 48 - protein, 753 aa, Escherichia coli
MSQHNEKNPHQHQSPLHDSSEAKPGMDSLAPEDGSHRPAAEPTPPGAQPTAPGSLKAPDTRNEKLNSLEDVRKGSENYALTTNQGVRIADDQNSLRAGSRGPTLLEDFILREKITHFDHERIPERIVHARGSAAHGYFQPYKSLSDITKADFLSDPNKITPVFVRFSTVQGGAGSADTVRDIRGFATKFYTEEGIFDFVGNNTPIFFIQDAHKFPDFVHAVKPEPHWAIPQGQSAHDTFWDYVSLQPETLHNVMWAMSDRGIPRSYRTMEGFGIHTFRLINAEGKATFVRFHWKPLAGKASLVWDEAQKLTGRDPDFHRRELWEAIEAGDFPEYELGFQLIPEEDEFKFDFDLLDPTKLIPEELVPVQRVGKMVLNRNPDNFFAENEQAAFHPGHIVPGLDFTNDPLLQGRLFSYTDTQISRLGGPNFHEIPINRPTCPYHNFQRDGMHRMGIDTNPANYEPNSINDNWPRETPPGPKRGGFESYQERVEGNKVRERSPSFGEYYSHPRLFWLSQTPFEQRHIVDGFSFELSKVVRPYIRERVVDQLAHIDLTLAQAVAKNLGIELTDDQLNITPPPDVNGLKKDPSLSLYAIPDGDVKGRVVAILLNDEVRSADLLAILKALKAKGVHAKLLYSRMGEVTADDGTVLPIAATFAGAPSLTVDAVIVPCGNIADIADNGDANYYLMEAYKHLKPIALAGDARKFKATIKIADQGEEGIVEADSADGSFMDELLTLMAAHRVWSRIPKIDKIPA

SEQ ID NO: 49 - DNA, 2023 bp, Bacillus subtilis
  gene (1)..(2023): lctP
cggtgcagatgacgtgtacatcggtgtgccggctgtcgtgaatcgcggagggatcgcaggtatcactgagctgaacttaaatgagaaagaaaaagaacagttccttcacagcgccggcgtccttaaaaacattttaaaacctcattttgcagaacaaaaagtcaactaaccgcaactttagagtaaagggctgattgtcaatgtgggagcagttgtatgatccgtttggaaacgagtatgtgagcgcacttgtggcgctcactccgattctcttttttcttttggctttaactgttttgaaaatgaaaggcattcttgcggcatttcttaccctagccgtcagtttcttcgtctccgtttgggcatttcatatgccggttgaaaaagcgatttcttctgttttgttaggaatcgggagcgggctgtggcccattggctacatcgtcctgatggcggtgtggctgtataaaatcgccgtgaaaaccgggaaatttaccattattcggtccagcattgccggcatttcgcctgaccaacgattacagctattattaattggtttttgttttaacgcgtttttagaaggcgcggccggttttggtgttccgattgcgattagtgcggcgctgctcgtcgaacttggttttaaaccgttaaaagcggcggcgctctgcttgattgcaaacgctgcctccggagcctttggggcgattgggattcctgtcatcacaggggcgcagattggtgatttgtctgctcttgagctgtctcggacattaatgtggacactgccgatgatctcatttttaataccattcctgcttgtattcttattagaccgaatgaaaggaatcaaacagacatggcccgctcttctggttgtgagcggtgggtatacagcggttcagacactgacaatggcggtgctcgggccggaattagcaaacattttggcggccttattcagcatgggcgggcttgccttcttcctccgcaaatggcagccgaaagagatttaccgcgaggaaggggccggcgatgctggtgagaaaaaggcataccgtgccgctgacattgcgagagcgtggtctcctttctacattttaactgcggcgatcaccatctggagccttcctgccttcaaagcgcttttccaagaaggcgggctgttatatcaatcaacgctcctgttcaaaatgccttttctgcatcagcaaattatgaaaatgccgccgattgcgccatctgccatgccgttagatgcagtctttaaagttgatctgctgtcagcgactggtacagcgattttagcggcggtcatcgtgacagggctggtcagcaaaaagttctcctctcgggatgcctttgcttccttgaaggagacgggaaaagagctgtgggtgccgattatgacgatctgcttcgtgatggggtttgccaatctggccaacttcgcagggctcagctcttcaattgggttagcattggcgagaacaggagacctgttcccgtttgtcagtcctgttctcggctggattggcgtgttcatcaccggttctgttgtcagcaataatgctttgttcggccatttgcaggttgtcacgggagcgcagatcggtgcgggttcagatttgctgttagccgcaaatacggcgggcggtgtcatggcgaaacttgtttctcctcaatctatcgccatcgctgccgcagcggtcggccaaacaggcaaggaatctaaactgtttaaaaggacagtgccgtacagcctgattctgttattgatcatttgtatatggacgtttactcttgcaagattaggagtgtaatagaaaaaagcagtacatgcccagcatgtactgctttttttatgttaatttgctttctttgtcatttcggctgttgttttcactctggcttttcccataaataaaatggtaaccgccgcgaggacaattggaatcaaagcgagcaaaaagacatacgtgatacttgatgacatcgcgtcaataatgcgattcaggat

SEQ ID NO: 50 - protein, 541 aa, Bacillus subtilis
MWEQLYDPFGNEYVSALVALTPILFFLLALTVLKMKGILAAFLTLAVSFFVSVWAFHMPVEKAISSVLLGIGSGLWPIGYIVLMAVWLYKIAVKTGKFTIIRSSIAGISPDQRLQLLLIGFCFNAFLEGAAGFGVPIAISAALLVELGFKPLKAAALCLIANAASGAFGAIGIPVITGAQIGDLSALELSRTLMWTLPMISFLIPFLLVFLLDRMKGIKQTWPALLVVSGGYTAVQTLTMAVLGPELANILAALFSMGGLAFFLRKWQPKEIYREEGAGDAGEKKAYRAADIARAWSPFYILTAAITIWSLPAFKALFQEGGLLYQSTLLFKMPFLHQQIMKMPPIAPSAMPLDAVFKVDLLSATGTAILAAVIVTGLVSKKFSSRDAFASLKETGKELWVPIMTICFVMGFANLANFAGLSSSIGLALARTGDLFPFVSPVLGWIGVFITGSVVSNNALFGHLQVVTGAQIGAGSDLLLAANTAGGVMAKLVSPQSIAIAAAAVGQTGKESKLFKRTVPYSLILLLIICIWTFTLARLGV

SEQ ID NO: 51 - DNA, 1363 bp, Bacillus subtilis
  gene (1)..(1363): ldh
ttaagcggttctcttccccattgagggcaaggctagacgggacttaccgaaagaaaccatcaatgatggtttcttttttgttcataaatcagacaaaacttttctcttgcaaaagtttgtgaagtgttgcacaatataaatgtgaaatacttcacaaacaaaaagacatcaaagagaaacataccctgcaaggatgattaatgatgaacaaacatgtaaataaagtagctttaatcggagcgggttttgttggaagcagttatgcatttgcgttaattaaccaagggatcacagatgagcttgtggtcattgatgtaaataaagaaaaagcaatgggcgatgtgatggatttaccccacggaaaggcgtttgggctacaaccggtcaaaacatcttacggaacatatgaagactgcaaggatgctgatattgtctgcatttgcgccggagcaaaccaaaaacctggtgagacacgccttgaattagtagaaaagaacttgaagattttcaaaggcatcgttagtgaagtcatggcgagcggatttgacggcattttcttagtcgcgacaaatccggttgatatcctgacttacgcaacatggaaattcagcggcctgccaaaagagcgggtgattggaagcggcacaacacttgattctgcgagattccgtttcatgctgagcgaatactttggcgcagcgcctcaaaacgtacacgcgcatattatcggagagcacggcgacacagagcttcctgtttggagccacgcgaatgtcggcggtgtgccggtcagtgaactcgttgagaaaaacgatgcgtacaaacaagaggagctggaccaaattgtagatgatgtgaaaaacgcagcttaccatatcattgagaaaaaaggcgcgacttattatggggttgcgatgagtcttgctcgcattacaaaagccattcttcataatgaaaacagcatattaactgtcagcacatatttggacgggcaatacggtgcagatgacgtgtacatcggtgtgccggctgtcgtgaatcgcggagggatcgcaggtatcactgagctgaacttaaatgagaaagaaaaagaacagttccttcacagcgccggcgtccttaaaaacattttaaaacctcattttgcagaacaaaaagtcaactaaccgcaactttagagtaaagggctgattgtcaatgtgggagcagttgtatgatccgtttggaaacgagtatgtgagcgcacttgtggcgctcactccgattctcttttttcttttggctttaactgttttgaaaatgaaaggcattcttgcggcatttcttaccctagccgtcagtttcttcgtctccgtttgggcat

SEQ ID NO: 52 - protein, 321 aa, Bacillus subtilis
MMNKHVNKVALIGAGFVGSSYAFALINQGITDELVVIDVNKEKAMGDVMDLPHGKAFGLQPVKTSYGTYEDCKDADIVCICAGANQKPGETRLELVEKNLKIFKGIVSEVMASGFDGIFLVATNPVDILTYATWKFSGLPKERVIGSGTTLDSARFRFMLSEYFGAAPQNVHAHIIGEHGDTELPVWSHANVGGVPVSELVEKNDAYKQEELDQIVDDVKNAAYHIIEKKGATYYGVAMSLARITKAILHNENSILTVSTYLDGQYGADDVYIGVPAVVNRGGIAGITELNLNEKEKEQFLHSAGVLKNILKPHFAEQKVN

SEQ ID NO: 53 - DNA, 1246 bp, Bacillus subtilis
  gene (1)..(1246): opuAB
gaatacgtatctgactgagatttttgacgttgtgtccgatgcgaatattccaattgcggttgtggatgagaagcaaagaatgaaaggaattgtcgtaagaggcgcgctgattggcgcacttgccggcaataacgagtatatcaatgctgaaggcactaacgaacaaacacaagatccttctgcacaggaggtgaaataagatggatagactgcctagaatacctttagcagatattattgaccgttttgttgactggattacgatgacgtttggcggattcttcgacggaatcgctaacggattggccgcttttgtaaatggaattgtcaccggtcttggatttattccatctattttgttaacgattattttcgccgcacttgcgtggtggatcagtacaaggggaattgcgttatttacgttgattggattcctcctgatcgattatttaggatattgggacccaatgctgcaaacattggcgcttgttttgacatctgtgattatttcgattgtggtcggggttccgatcggaatttgggcgtctcagaaagaaacggttcgccgtattgtaacgccaattcttgatttaatgcagacaatgcctgctttcgtatatctattgccggcgattttcttctttaacatcggtgttgtgccgggtgttgttgcatctgttatcttcgcgatgccgccgacaatccgcatgactgtcctcggtattaaacaagtgccggcggatttgattgaagcgactgaggcattcggttctacaacagctcagcgtttgtttaaagttcagctcccgcttgcgacaaaaacgattttggccgggatcaaccagagcatcatgcttgcgttatcaatggttgttatcgccgcaatggtcggtgcaccgggacttggttctgaagtatacagtgccgttacacagctgaaaaccggggtcggagtagaggccggtatcgccatcgttatcgttgccattaccctggaccgtattactcaaaatattaaagtgaagaagaaaagcagggggaatgcctgatgcttaaaaaaatcataggcatcggcgtttccgccatgctggcgctctcacttgcggcttgcggttcagaaaacgatgaaaatgcatccgcagcagaacaagtgaataagaccatcatcgggattgaccccggttcagggattatgtccctgactgacaaagcgatgaaggattatgacctgaatgactggacattg

SEQ ID NO: 54 - protein, 282 aa, Bacillus subtilis
MDRLPRIPLADIIDRFVDWITMTFGGFFDGIANGLAAFVNGIVTGLGFIPSILLTIIFAALAWWISTRGIALFTLIGFLLIDYLGYWDPMLQTLALVLTSVIISIVVGVPIGIWASQKETVRRIVTPILDLMQTMPAFVYLLPAIFFFNIGVVPGVVASVIFAMPPTIRMTVLGIKQVPADLIEATEAFGSTTAQRLFKVQLPLATKTILAGINQSIMLALSMVVIAAMVGAPGLGSEVYSAVTQLKTGVGVEAGIAIVIVAITLDRITQNIKVKKKSRGNA

SEQ ID NO: 55 - DNA, 1778 bp, Bacillus subtilis
  gene (1)..(1778): phoA
tttttatttaaacaaacatatcatgcaaagacagagaggtaaagatttttctgaaaaatgaatgctttacatcaaataaggcaagataacgaaaagcgttttttcatttccttacaaggctttcattattgtttacatgatcaacagccgcatttaacaaagtttccctaacatgataaacggaatacattaaaggaggcatgaaaaaaatgagtttgtttcaaaatatgaaatcaaaacttctgccaatcgccgctgtttctgtccttacagctggaatctttgccggagctgagcttcagcaaacagaaaaggccagcgccaaaaaacaagacaaagctgagatcagaaatgtcattgtgatgataggcgacggcatggggacgccttacataagagcctaccgttccatgaaaaataacggtgacacaccgaataacccgaagttaacagaatttgaccggaacctgacaggcatgatgatgacgcatccggatgaccctgactataatattacagattcagcagcagccggaacagcattagcgacaggcgttaagacatataacaatgcaattggcgtcgataaaaacggaaaaaaagtgaaatctgtacttgaagaggccaaacagcaaggcaagtcaacagggcttgtcgccacgtctgaaattaaccacgccactccagccgcatatggcgcccacaatgaatcacggaaaaacatggaccaaatcgccaacagctatatggatgacaagataaaaggcaaacataaaatagacgtgctgctcggcggcggaaaatcttattttaaccgcaagaacagaaacttgacaaaggaattcaaacaagccggctacagctatgtgacaactaaacaagcattgaaaaaaaataaagatcagcaggtgctcgggcttttcgcagatggagggcttgctaaagcgctcgaccgtgacagtaaaacaccgtctctcaaagacatgacggtttcagcaattgatcgcctgaaccaaaataaaaaaggatttttcttgatggtcgaagggagccagattgactgggcggcccatgacaatgatacagtaggagccatgagcgaggttaaagattttgagcaggcctataaagccgcgattgaatttgcgaaaaaagacaaacatacacttgtgattgcaactgctgaccatacaaccggcggctttaccattggcgcaaacggggaaaagaattggcacgcagaaccgattctctccgctaagaaaacacctgaattcatggccaaaaaaatcagtgaaggcaagccggttaaagatgtgctcgcccgctatgccaatctgaaagtcacatctgaagaaatcaaaagcgttgaagcagctgcacaggctgacaaaagcaaaggggcctccaaagccatcatcaagatttttaatacccgctccaacagcggatggacgagtaccgatcataccggcgaagaagtaccggtatacgcgtacggccccggaaaagaaaaattccgcggattgattaacaatacggaccaggcaaacatcatatttaagattttaaaaactggaaaataaaaaggatgcagaacgctgcatcctcttttttataaatatctgcggtgaacaccttttccgtcgtacgaaaacatgacttccttatcctcaagcacagactcaatgtgcacgcttcttccccaaagctggaataaataaggaagcactttctccaaatatttcaaatccaactctatgccctcataccaatgc

SEQ ID NO: 56 - protein, 461 aa, Bacillus subtilis
MKKMSLFQNMKSKLLPIAAVSVLTAGIFAGAELQQTEKASAKKQDKAEIRNVIVMIGDGMGTPYIRAYRSMKNNGDTPNNPKLTEFDRNLTGMMMTHPDDPDYNITDSAAAGTALATGVKTYNNAIGVDKNGKKVKSVLEEAKQQGKSTGLVATSEINHATPAAYGAHNESRKNMDQIANSYMDDKIKGKHKIDVLLGGGKSYFNRKNRNLTKEFKQAGYSYVTTKQALKKNKDQQVLGLFADGGLAKALDRDSKTPSLKDMTVSAIDRLNQNKKGFFLMVEGSQIDWAAHDNDTVGAMSEVKDFEQAYKAAIEFAKKDKHTLVIATADHTTGGFTIGANGEKNWHAEPILSAKKTPEFMAKKISEGKPVKDVLARYANLKVTSEEIKSVEAAAQADKSKGASKAIIKIFNTRSNSGWTSTDHTGEEVPVYAYGPGKEKFRGLINNTDQANIIFKILKTGK

SEQ ID NO: 57 - DNA, 1811 bp, Escherichia coli
  gene (1)..(1811): phoA
ggcgctgtacgaggtaaagcccgatgccagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtacatggagaaaataaagtgaaacaaagcactattgcactggcactcttaccgttactgtttacccctgtgacaaaagcccggacaccagaaatgcctgttctggaaaaccgggctgctcagggcgatattactgcacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgccttaccgcttaccgggcaatacactcactatgcgctgaataaaaaaaccggcaaaccggactacgtcaccgactcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaattcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgagacggtcgatctcgatgaagccgtacaacgggcgctggaattcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctggggctgaaataaaaccgcgcccggcagtgaattttcgctgccgggtggtttttttgctgttagcaaccagacttaatggcagatcacgggcgcatacgctcatggttaaaacatgaagagggatggtgctatgaaaataacattactggttaccttgcttttcggtctggtttttttaaccaccgtcggcgctgccgagagaacttt

SEQ ID NO: 58 - protein, 471 aa, Escherichia coli
VKQSTIALALLPLLFTPVTKARTPEMPVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 59 - DNA, 2068 bp, Bacillus subtilis
  gene (1)..(2068): phoD
gccgaatgggcggcttgcaaacgacaggatatgcagaaaacgcgtcattcagccagctttcggcatgtttcagcagcaggcttgtgagggaagacctgtttttaatgttcagcgcttacaatcagttcacacttcttcacagtcgtttaacaatgatttcctataatggagacgatcaatgaggagagaggggatcttgaatggcatacgacagtcgttttgatgaatgggtacagaaactgaaagaggaaagctttcaaaacaatacgtttgaccgccgcaaatttattcaaggagcggggaagattgcaggactttctcttggattaacgattgcccagtcggttggggcctttgaagtaaatgctgcgcctaacttctcaagctatccgtttacattaggggtagcgtcaggtgatccgctttctgacagtgtcgtgctttggacacgtctggcaccagatccgctgaatggcgggggaatgccgaagcaagcagtgcctgtcaaatgggaggtcgcaaaggatgagcatttccgcaaaatcgtaagaaaaggcactgaaatggctaaaccaagtcttgcccattccgttcatgtggaagcggacgggcttgagccaaacaaagtgtactattaccgtttcaaaaccggccatgaactaagccctgtcggaaaaacaaaaacactgcctgcccctggcgccaatgtgccgcaaatgacctttgcatttgcttcctgccagcaatatgagcacggatattatacggcctacaagcatatggcaaaggaaaagcttgatcttgtcttccatctcggtgattacatatatgaatacggcccaaatgaatatgtatcaaaaacaggcaatgtccgcacgcacaacagcgcggaaatcattacgctgcaagactatcggaaccgtcacgctcaataccgttctgacgccaatcttaaagctgcacatgcggcattcccttgggttgtaacttgggatgaccatgaagttgaaaacaactatgccaataaaatacctgaaaaaggacagtcggttgaagcattcgtgctccgccgcgcggcagcataccaggcatactatgaacatatgccgctccgcatttcctctttgccgaatggcccagatatgcagctgtaccggcactttacgtacggaaacctggcgtccttcaatgtgcttgatacccgccagtacagggacgaccaagccaataacgacggcaataagccgccttctgatgaatcgcgcaatccgaatcggactctgcttggaaaagaacaggagcagtggctcttcaacaatttgggcagctcgacagcccactggaatgtgctggcgcagcagattttctttgccaagtggaatttcgggacaagcgcaagcccgatctacagcatggattcgtgggacggctacccggctcagcgcgaacgggttatcaatttcattaaaagcaaaaatctgaataatgttgtcgtactgacaggtgatgtacacgcaagctgggcatcgaatttgcatgtcgatttcgagaagacaagttcaaaaatattcggagccgaatttgttggaacctcgatcacttccggagggaacggagcggataagcgagcggatacggaccaaattttaaaagaaaatccgcacatccagttctttaacgactatcgcggatatgtccgctgtacagtgacgcctcaccaagtggaaagccgattatcgggtgatgccatttgtgaccgagccgggcgcagccatttccacgcgggcttcattcgtttaccagaaagaccaaaccgggttgagaaaggtatcatccacaacaatccaaggcggggtgaagcaatccgatgaggtcgaagaggatcgtttcttttcgcacaacaaagcccacgaaaaacaaatgattaagaagcgtgcaaaaatcacgaattaaggagtggaaattatgttttcaaacattggaataccgggcttgattctcatcttcgtcatcgccattattatttttggcccttccaagctgccggaaatcgggcgtgccgcgaaac

SEQ ID NO: 60 - protein, 556 aa, Bacillus subtilis
MAYDSRFDEWVQKLKEESFQNNTFDRRKFIQGAGKIAGLSLGLTIAQSVGAFEVNAAPNFSSYPFTLGVASGDPLSDSVVLWTRLAPDPLNGGGMPKQAVPVKWEVAKDEHFRKIVRKGTEMAKPSLAHSVHVEADGLEPNKVYYYRFKTGHELSPVGKTKTLPAPGANVPQMTFAFASCQQYEHGYYTAYKHMAKEKLDLVFHLGDYIYEYGPNEYVSKTGNVRTHNSAEIITLQDYRNRHAQYRSDANLKAAHAAFPWVVTWDDHEVENNYANKIPEKGQSVEAFVLRRAAAYQAYYEHMPLRISSLPNGPDMQLYRHFTYGNLASFNVLDTRQYRDDQANNDGNKPPSDESRNPNRTLLGKEQEQWLFNNLGSSTAHWNVLAQQIFFAKWNFGTSASPIYSMDSWDGYPAQRERVINFIKSKNLNNVVVLTGDVHASWASNLHVDFEKTSSKIFGAEFVGTSITSGGNGADKRADTDQILKENPHIQFFNDYRGYVRCTVTPHQVESRLSGDAICDRAGRSHFHAGFIRLPERPNRVEKGIIHNNPRRGEAIR

SEQ ID NO: 61 - DNA, 1300 bp, Bacillus subtilis
  gene (1)..(1300): pstS
acaaaattgaagaaaatgcagaaaacacaacgtcttctgataactaaaaaaaacgctcacatgatgtgggcgttttttttatacaaaaaaacgcactgatttacaaaaccttaacattcggttcaaaccctttttacatagaacctttactctatacgtgtaggacaaattacacattatacgcagggggaattcaggaaatgaaaaaaaacaaattggtgcttatgcttctgatggctgcttttatgatgattgcagctgcatgcggaaatgcaggagaaagtaaaaaatcgaatagcgattctgcaaaaggagaagaaaaggcatcaggttctttaaccatttctggttcatcggcaatgcagccactagtacttgcagccgcggaaaaatttatggaagaaaaccctgatgctgatatacaggtacaagctggcggttctggaacagggctttctcaagtctctgaaggagctgttcagatcgggaattcagacgtttttgctgaagaaaaagaaggcatcgatgcaaaagcgcttgtcgatcatcaagtcgctgtagttggtatggcagcagctgtaaaccctgatgctggtgtaaaggatatttcaaaagatgaattgaaaaaaatcttcactggcaaaatcaaaaactggaaagagctcggcggcaaagaccaaaagattacacttgtaaaccgccctgattcttccggcacacgtgctacatttgtgaaatatgcgcttgacggagcggagcctgcagaaggcattactgaagattcctccaacacggtgaaaaagatcattgctgatactccgggagcgattggatatcttgcattctcatatttaacagatgacaaagtaactgctctatctattgacggtgttaaacctgaagcgaaaaatgtagcaactggcgagtatccgatctgggcttatcaacattcttatacaaaaggtgaagcgacaggtttagctaaagaatttcttgattatctaaagagtgaagacattcaaaaaagcattgttactgaccaaggctacattccggttactgatatgaaagtgacacgtgatgccaacgggaagcaaagctgatcttaataaaaagaggagtgcggggacaaccgtttctcctcttaacacattcataggagtgtaaaacaatgataaacaatagagaaaatatgtctgtgagcgagcgcttaatcagctctaggcaaaatcggcaattggatgaagttcggggaagaatgatagtcacagcttgtgcactgattatgattgcggcatct

SEQ ID NO: 62 - protein, 300 aa, Bacillus subtilis
MKKNKLVLMLLMAAFMMIAAACGNAGESKKSNSDSAKGEEKASGSLTISGSSAMQPLVLAAAEKFMEENPDADIQVQAGGSGTGLSQVSEGAVQIGNSDVFAEEKEGIDAKALVDHQVAVVGMAAAVNPDAGVKDISKDELKKIFTGKIKNWKELGGKDQKITLVNRPDSSGTRATFVKYALDGAEPAEGITEDSSNTVKKIIADTPGAIGYLAFSYLTDDKVTALSIDGVKPEAKNVATGEYPIWAYQHSYTKGEATGLAKEFLDYLKSEDIQKSIVTDQGYIPVTDMKVTRDANGKQS

SEQ ID NO: 63 - DNA, 1441 bp, Escherichia coli
  gene (1)..(1441): pstS
agaatgaaaatttttctgtcatctcttcgttattaatgtttgtaattgactgaatatcaacgcttatttaaatcagactgaagactttatctctctgtcataaaactgtcatattccttacatataactgtcacctgtttgtcctattttgcttctcgtagccaacaaacaatgctttatgaatcctcccaggagacattatgaaagttatgcgtaccaccgtcgcaactgttgtcgccgcgaccttatcgatgagtgctttctctgtgtttgcagaagcaagcctgacaggtgcaggtgcaaccttccctgcgccggtgtatgccaaatgggctgacacttaccagaaagaaaccggtaataaagttaactaccagggtatcggttcttccggtggcgtaaaacagattatcgctaataccgttgattttggtgcctctgacgcgccgctgtctgacgaaaaactggctcaggaaggtctgttccagttcccgaccgtgattggcggcgtggtgctggcggttaacattccagggctgaagtctggcgaactggtgctggatggtaaaaccctcggcgacatctacctgggcaaaatcaagaagtgggatgatgaagccatcgccaaactgaatccgggtctgaaactgccttcacaaaacattgctgtagtacgccgcgcagatggctccgggacttccttcgtcttcaccagctacctggcgaaagtgaacgaagagtggaaaaacaacgttggtactggctctaccgtaaaatggccgatcggtctgggcggtaaaggtaacgacggtatcgccgcgttcgttcagcgtctgccgggtgcaattggttatgttgaatatgcttacgcgaagcagaacaacctggcgtacaccaaactgatctccgctgatggtaaaccggttagtccgaccgaagaaaacttcgctaatgcagcaaaaggtgcagactggagcaaaaccttcgctcaggatctgaccaaccagaaaggcgaagatgcatggcctattacctctaccacgttcattctgatccacaaagatcagaagaaaccagaacaaggcacagaagtgctgaaattcttcgactgggcgtacaaaaccggggctaaacaggcgaacgacctggattacgccagcctgccggatagtgtagttgaacaggttcgcgctgcgtggaagaccaatattaaagacagtagcggtaagccgctgtactaataaaactccaggccgggtacggtgttttacgccgcatccggcattacaaaatgactttgtaaacgcgtttaactgaagagtaacttatggctgcaaccaagcctgcttttaacccaccgggtaaaaagggcgacataattttcagcgtgctggtaaaactggcggcgctgattgtgctattgatgttgggtggcattatt

SEQ ID NO: 64 - protein, 346 aa, Escherichia coli
MKVMRTTVATVVAATLSMSAFSVFAEASLTGAGATFPAPVYAKWADTYQKETGNKVNYQGIGSSGGVKQIIANTVDFGASDAPLSDEKLAQEGLFQFPTVIGGVVLAVNIPGLKSGELVLDGKTLGDIYLGKIKKWDDEAIAKLNPGLKLPSQNIAVVRRADGSGTSFVFTSYLAKVNEEWKNNVGTGSTVKWPIGLGGKGNDGIAAFVQRLPGAIGYVEYAYAKQNNLAYTKLISADGKPVSPTEENFANAAKGADWSKTFAQDLTNQKGEDAWPITSTTFILIHKDQKKPEQGTEVLKFFDWAYKTGAKQANDLDYASLPDSVVEQVRAAWKTNIKDSSGKPLY

SEQ ID NO: 65 - DNA, 1123 bp, Bacillus subtilis
  gene (1)..(1123): purC
gcgagcttgtggaagcggaagagaaaatcacgtcacgtctttctccagaaaaaattgctgactgctttgattacaattaccatctgaaaaatgttgatctgatctttgaacgtttaggtttagcatagaagaagcttttacggcttcttctaagccgccgcagtttgaaaattcccaacattcgggttaggaggccttccgtgaatattgtgaagaatgaacttttatacgaaggaaaagcaaaaaagatctacaaaaccgatgacgaaaacacgctgtatgtcgtgtataaagactccgccactgcctttaacggcgagaaaaaagcagaaatcagcggaaaagggcgcttaaataatgaaatttcaagcctcattttcaaacaccttcatgctaagggcattaacaatcattttatcgagcgcatttcggaaacggagcagctcattaaaaaggtaacgattgtgccgcttgaagtcgtggtaagaaatgttgtggcaggaagcatgtccaaacgtctcggcattccagaaggcacggagcttgagcagccgattatcgagttttactacaaggatgacgcgctgggtgatccgctcatcacagaagatcatatttggcttttgaaggcggcgactcctgagcaggtagaaaccattaagtccattacaacaatagtaaatgaagagcttcaaagcatcttcgacgattgtcatgtcagattaatagatttcaagcttgaattcggtttagatgcagaagggcaagtgcttttggcggatgaaatatctcctgacacatgccgcttgtgggataaagaaacgaacgaaaagctggacaaagatttattcagacgcaatctgggaagcttaaccgacgcatacgaagagattttcaatagactgggaggcattcatcatgtataaagtaaaagtttatgtcagcttaaaagaaagtgtactagatccacaagggagcgctgtccagcatgccttgcacagtatgacttacaacgaagttcaagatgtgcgcatcgggaaatacatggagcttaccattgaaaaatctgaccgtgatcttgacgtgctagtgaaagaaatgtgcgaaaaacttcttgcgaaca

SEQ ID NO: 66 - protein, 241 aa, Bacillus subtilis
VNIVKNELLYEGKAKKIYKTDDENTLYVVYKDSATAFNGEKKAEISGKGRLNNEISSLIFKHLHAKGINNHFIERISETEQLIKKVTIVPLEVVVRNVVAGSMSKRLGIPEGTELEQPIIEFYYKDDALGDPLITEDHIWLLKAATPEQVETIKSITTIVNEELQSIFDDCHVRLIDFKLEFGLDAEGQVLLADEISPDTCRLWDKETNEKLDKDLFRRNLGSLTDAYEEIFNRLGGIHHV

SEQ ID NO: 67 - DNA, 985 bp, Bacillus subtilis
  gene (1)..(985): purN
tctttcttgcaagagtacggcaagctaaaggaagaagacatgttcaacgtctttaatatgggtatcggttttgttttggcagtcaaagaagagcatctgacagatgtgatcggaacgcttgaaagccatggcgaaaaagcctatttaatcgggcgtgtgaaaaaaggtgaaggcgtcacattcggcggtgcggcactttcatgaaaaagtttgcggtatttgcatcaggaaacggttcaaacttcgaagccatcgtcacgcgtttgaaggaggagaactgggatgcgtcacgagcgctcctcgtttgcgacaaaccgcaggcgaaagtcatcgaacgggcggaacgattccacattccatccttcgcatttgagccgaagtcttatgaaaacaaggctgcatttgaacaagccatcattgaacagcttcgtcttcacgaggttgaattgattgctcttgccggctatatgaggctgatcggtgatacgctccttcaagcatatgggggaaaaatcattaacattcacccatcgcttcttccggcgtttcctggaatcgacgcagtcggacaggcgttccgggcgggtgtgaaggtggccggaatcaccgtgcattatgtcgatgaaggaatggatacaggtccgatcatcgctcaaaaggcaatcgaaattgatgaacatgatacattggaaacaatcgaacagcgaattcacaagcttgagcataaatggtatccgagtgtgattaaacagctattaggattaaataacagaggtgaaaaggcatgaccattaaacgtgcattaatcagtgtttcagataaaacaaatctcgttcctttcgtaaaagaactgacagagcttggcgttgaagtcatctccacaggcggaacgaaaaagcttcttcaagaaaacggtgtggatgtgatcggaatttctgaagtgacaggctttcctgaaattatggacggccggttaaaaacccta

SEQ ID NO: 68 - protein, 195 aa, Bacillus subtilis
MKKFAVFASGNGSNFEAIVTRLKEENWDASRALLVCDKPQAKVIERAERFHIPSFAFEPKSYENKAAFEQAIIEQLRLHEVELIALAGYMRLIGDTLLQAYGGKIINIHPSLLPAFPGIDAVGQAFRAGVKVAGITVHYVDEGMDTGPIIAQKAIEIDEHDTLETIEQRIHKLEHKWYPSVIKQLLGLNNRGEKA

SEQ ID NO: 69 - DNA, 1312 bp, Bacillus subtilis
  gene (1)..(1312): pyrB
tcttccgcaggcgaaggaagagcaggcagacacatctgaacaacatcatatttaaaaccttttaatgaaagtccagagaggcttggaagggttatgaagagaaggaagcttcaatgctgccctctatttaaccataccccgagtctatcttagaccggggttttttttcagccttaagttgaaaagagaggggaaagaacatgaagcatttaacgacgatgagtgaacttagcactgaggaaatcaaagatttgcttcaaacagcacaagagctcaaaagcggaaaaacagacaatcagcttacaggaaagtttgcagcaaacctgtttttcgaaccgagcacgagaacgcggttcagctttgaggtcgcagaaaaaaagctgggcatgaatgtgcttaaccttgatggaacaagcacaagcgtgcaaaaaggcgaaaccttatatgacacgatccggacgcttgaatcaatcggtgtggacgtctgcgtcatcaggcacagtgaggatgagtattatgaagagcttgtcagccaggtgaacattccgattctgaatgcgggagacgggtgcggccagcatccaacacaatcactgcttgatttaatgacgatttatgaagagttcaatacgtttaaagggcttaccgtctccattcacggcgacatcaagcatagcagagtggcaaggtcaaatgcggaagtgttgacaagattgggtgcccgggtcctattttccggcccttcggaatggcaggatgaagaaaatacattcggcacgtatgtctcaatggatgaagcagttgagtcttccgatgttgtcatgctgctgcgcattcaaaatgaacgacatcagtccgctgtcagtcaggaaggctatttaaacaaatacggcttgaccgtagaacgggctgagcgtatgaagcggcatgcgatcatcatgcatcctgctccggtaaacagaggagtggagattgatgacagcttagtagaaagcgaaaaatcaagaatcttcaagcaaatgaaaaatggcgtatttatcagaatggcagtgatacagtgtgccttacaaaccaatgtgaaaagaggagaagcagcgtatgtcatatctcattaaaaacggctggatactaaacgaaaatggtgaaaaaacacaagcggatatccgagtgactggagaaaccatcaccgcaatcggcaagcttgatgcaacggataatgaaacggtaattgatgcaaaaggtttgctcgtttcacctgggtttgttgatctccacgtgcatttcagagagccgggcggagagaaaaaagaaa

SEQ ID NO: 70 - protein, 304 aa, Bacillus subtilis
MKHLTTMSELSTEEIKDLLQTAQELKSGKTDNQLTGKFAANLFFEPSTRTRFSFEVAEKKLGMNVLNLDGTSTSVQKGETLYDTIRTLESIGVDVCVIRHSEDEYYEELVSQVNIPILNAGDGCGQHPTQSLLDLMTIYEEFNTFKGLTVSIHGDIKHSRVARSNAEVLTRLGARVLFSGPSEWQDEENTFGTYVSMDEAVESSDVVMLLRIQNERHQSAVSQEGYLNKYGLTVERAERMKRHAIIMHPAPVNRGVEIDDSLVESEKSRIFKQMKNGVFIRMAVIQCALQTNVKRGEAAYVISH

SEQ ID NO: 71 - DNA, 1702 bp, Bacillus subtilis
  gene (1)..(1702): pyrP
atctcgtcgccatttatgaaaacgaataatagatcacctttttaagcaatccagagaggttgcaaagaggtgcacaacaaaggcccaaaaagtattcggcaggtctttgtatgcctctttgcgtaaaaaagcaaagaggtttttttatacagtcattgagtcatcctaaaatgaaagtcaacaatcagggggaaatcatcatgagtaagaaaaaagtaaatttaggggtcagggatgtcccgacacctttctcttgggtttcattcagccttcagcatttgtttgccatgtttggctcaaccattttggttccgaagctcgtcggaatgagtcctgctgtggcgttggtgacaagcggcatcggaacactggcgtaccttcttattaccaaaggacaaattccggcgtatctcggttcatccttcgcctttatttctccgatcattctggtaaaagcgaccggcgggccgggagcggcaatggttggagcgtttcttgcagggctggtgtacgggctgattgccttattgattaggcagcttggaacaggatggctgatgaagattctcccgcctgtagtcgtagggcctgttattatcgtcatcgggctgggactggcaagcactgcagtaaacatggcgatgtatgctgatccgaacgcgagtgagttggtctacagcttaaagcactttagtgtcgcaggagttacgctggcaattacgattatttgtgcgattttcttacgagggtttttaagcctgattccggttctgatcggaatcatcggcggatacctgtttgcccttactcaagggattgtcaacttccagccggtgcttgacgcgaaatggtttgcagtgcctgaatttatcattccgttcaaagattatcaccgtcagttacgctcggcatcgcagccgcaatggttcctgtcgcatttgtcacaatgtcagagcatatcggccaccaatggtgctgagagcaaggttgtcggacaagacttcataaaagcaggtcttcatcgctctattatgggtgacagtgtggcgacaatcctcgcttccctgatcggcggccctccgacaacgacttacggagaaaacattggcgtgctggccatcacaagagtattcagcgtctttgtcatcgggggcgcggcagtgattgccctttgcttcggctttatcggcaaaatttcagcgctgatcagttcagtgccgtcagcggtcatgggaggcgtctccttcctgctgttcggaatcattgcttcaagcggcctgagaatgctgattgacaacaaaattgattatgaaaacaacagaaacctcattattacatcagttatccttgtcatcggtgtaggaggcgcttttatccaagtgtctcagggcggattccaagtgtcaggaatggcgcttgccgcaattgtcggtgtcatcttaaacctgattcttccgcaggcgaaggaagagcaggcagacacatctgaacaacatcatatttaaaaccttttaatgaaagtccagagaggcttggaagggttatgaagagaaggaagcttcaatgctgccctctatttaaccataccccgagtctatcttagaccggggttttttttcagccttaagttgaaaagagaggggaaagaacatgaagcatttaacgacgatgagtgaacttagcactgaggaaatcaaagatt

SEQ ID NO: 72 - protein, 434 aa, Bacillus subtilis
MSKKKVNLGVRDVPTPFSWVSFSLQHLFAMFGSTILVPKLVGMSPAVALVTSGIGTLAYLLITKGQIPAYLGSSFAFISPIILVKATGGPGAAMVGAFLAGLVYGLIALLIRQLGTGWLMKILPPVVVGPVIIVIGLGLASTAVNMAMYADPNASELVYSLKHFSVAGVTLAITIICAIFLRGFLSLIPVLIGIIGGYLFALTQGIVNFQPVLDAKWFAVPEFIIPFKDYHRQLRSASQPQWFLSHLSQCQSISATNGAESKVVGQDFIKAGLHRSIMGDSVATILASLIGGPPTTTYGENIGVLAITRVFSVFVIGGAAVIALCFGFIGKISALISSVPSAVMGGVSFLLFGIIASSGLRMLIDNKIDYENNRNLIITSVILVIGVGGAFIQVSQGGFQVSGMALAAIVGVILNLILPQAKEEQADTSEQHHI

SEQ ID NO: 73 - DNA, 1192 bp, Bacillus subtilis
  gene (1)..(1192): sigB
tgtggcggatgaaggagacagctttgactttgatcaaaagcagcaggatctagggccgtacacaccttcgcacacagttgatcaattatcagaaggagggctcggtctatatttaatggaaacgctcatggatgaagtcagagtgcaaaaccactccggcgtcaccgtagcgatgacaaagtatttaaatggggagcgagttgatcatgacacaaccatcaaaaactacgaaactaactaaagatgaagtcgatcggctcataagcgattaccaaacaaagcaagatgaacaagcgcaggaaacgcttgtgcgggtgtatacaaatctggttgacatgcttgcgaaaaaatactcaaaaggcaaaagcttccacgaggatctccgccaggtcggcatgatcgggctgctaggcgcgattaagcgatacgatcctgttgtcggcaaatcgtttgaagcttttgcaatcccgacaatcatcggtgaaattaaacgtttcctcagagataaaacatggagcgttcatgtgccgagacgaattaaagaactcggtccaagaatcaaaatggcggttgatcagctgaccactgaaacacaaagatcgccgaaagtcgaagagattgccgaattcctcgatgtttctgaagaagaggttcttgaaacgatggaaatgggcaaaagctatcaagccttatccgttgaccacagcattgaagcggattcggacggaagcactgtcacgattcttgatatcgtcggatcacaggaggacggatatgagcgggtcaaccagcaattgatgctgcaaagcgtgcttcatgtcctttcagaccgtgagaaacaaatcatagaccttacgtatattcaaaacaaaagccaaaaagaaactggggacattctcggtatatctcaaatgcacgtctcgcgcttgcaacgcaaagctgtgaagaagctcagagaggccttgattgaagatccctcgatggagttaatgtaatgatccaggttgaagaaaacgagcatattcaaactctagtatatcaactgaataaagaagggaaatcgatttgcggtgacagtttttttatgaaagctgatgataaggagttaatttgcgcggttgctgacggactgggaagtggatcacttgctaacgaatcctctgcagccatcaaagacttagtggaaaactat

SEQ ID NO: 74 - protein, 264 aa, Bacillus subtilis
LIMTQPSKTTKLTKDEVDRLISDYQTKQDEQAQETLVRVYTNLVDMLAKKYSKGKSFHEDLRQVGMIGLLGAIKRYDPVVGKSFEAFAIPTIIGEIKRFLRDKTWSVHVPRRIKELGPRIKMAVDQLTTETQRSPKVEEIAEFLDVSEEEVLETMEMGKSYQALSVDHSIEADSDGSTVTILDIVGSQEDGYERVNQQLMLQSVLHVLSDREKQIIDLTYIQNKSQKETGDILGISQMHVSRLQRKAVKKLREALIEDPSMELM

SEQ ID NO: 75 - DNA, 730 bp, Bacillus subtilis
  gene (1)..(730): tnrA
ctccatgattatccttcctcctcgtttgattcgttacataaaggataaaacgcgcttgggtgttacgcattattcttgttaatttttcttccgtattttttcaaaaatcttctatttgatgttagaaaatatgacaagcgtcaggttttttctctttgttttttatatactattatcaaaagacatccaggggtggaaaaatgaccacagaagatcattcttataaagacaaaaaagtgatttcaatcggaattgtgagtgaattgacaggattgtccgtaagacagatcaggtattatgaggaacgaaagctcatttacccgcagcgttcttcaagggggacaagaaaatactcctttgccgatgtggagcggctgatggatatcgccaataagcgtgaagacggcgtacagacggcagagattttaaaggatatgcgcaaaaaagaacagatgttaaaaaacgatccgcaagtgcggaaaaaaatgctggaggggcagcttaatgctcactttcggtacaaaaaccgttaaataataaaagtccggctcgcagttgagacggactttttacgtttataatgagtgaatgaatgcagtggcaatgccaaagtagattaataaagataaaatatcgtttaacgttgtaatcagcggtccagaagcaatcgccgggtctactttcagtttgtgcaaaataatagggataatcgttcctgacatcgtgccga

SEQ ID NO: 76 - protein, 110 aa, Bacillus subtilis
MTTEDHSYKDKKVISIGIVSELTGLSVRQIRYYEERKLIYPQRSSRGTRKYSFADVERLMDIANKREDGVQTAEILKDMRKKEQMLKNDPQVRKKMLEGQLNAHFRYKNR

SEQ ID NO: 77 - DNA, 712 bp, Bacillus subtilis
  gene (1)..(712): trxA
taatgattattggggcaattgtagtgatcggggccattatcagatttgtgttttaaaaaaagagcatatcccattcaaccatataaaaatgagtaaaccggctgtgatcaggaaaaaataatttgtaagcattaaaatagcgtgaacgaatgggagatgctatactaaaaatcatcatttcacattggaggaattcaataatggctatcgtaaaagcaactgatcaatctttctcagctgaaacaagcgaaggcgtcgtactggcagacttctgggctccttggtgcggaccttgtaaaatgattgcacctgttcttgaagaattggatcaagaaatgggagacaaactgaaaatcgtaaaaatcgatgtagacgaaaaccaagaaactgccggaaaatacggcgttatgagcatcccgactcttcttgtgttaaaagacggagaagtagttgaaacttcagtcggcttcaaaccaaaagaagcgcttcaagagcttgtaaacaaacatctttaattttccgctgcttacatgccagagcgattccgattgagggatcgctttttttattcgccaattgtgaaccatagagaaaaacaaacgttcgtgttaaactggaaataaaggattaaacataaggaagggtaacatatgaacaaacaactgaaagaaaaactcgccctccttcctgatcaaccgggttgttatctcat

SEQ ID NO: 78 - protein, 104 aa, Bacillus subtilis
MAIVKATDQSFSAETSEGVVLADFWAPWCGPCKMIAPVLEELDQEMGDKLKIVKIDVDENQETAGKYGVMSIPTLLVLKDGEVVETSVGFKPKEALQELVNKHL

SEQ ID NO: 79 - DNA, 730 bp, Escherichia coli
  gene (1)..(730): trxA
gggcttctacaaccttcggatgcagggcgaagtcggaaaacttctgttctgttaaatgtgttttgctcatagtgtggtagaatatcagcttactattgctttacgaaagcgtatccggtgaaataaagtcaacctttagttggttaatgttacaccaacaacgaaaccaacacgccaggcttattcctgtggagttatatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcgtaagggaatttcatgttcgggtgccccgtcgctaaaaactggacgcccggcgtgagtcatgctaacttagtgttgacttcgtattaaacataccttattaagtttgaatcttgtaatttccaacgcttcccgttttatcttaaatgcgaagtgaacagatttctggctcgtcactcaatccgtcttgtcgtttcagttctgcg

SEQ ID NO: 80 - protein, 109 aa, Escherichia coli
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 81 - DNA, 1081 bp, Bacillus subtilis
  gene (1)..(1081): ydjF (pspA)
tgtaaatcattgaagatgtttcttgaaagtctttggacttttgggggcatctttttttattgcttacatttttgttcaggtaaaaagttgtatattttttgtttgaaatgtcattttttattaaaaaagtgaaacttttaacgataataaatagtatatgtaacaagggttaccgaattagctttaggaggaacgatataatgagtataattggaagatttaaagatattatgtctgcgaatatcaatgctttattggacaaggctgaaaatccggagaaaatggtagatcaatatttaagaaatatgaatagcgatttggctaaggttaaggcagaaacagcagctgttatggctgaggagcagagagcaaaaagagaatatcacgaatgccaggcggatatggaaaagatggaaagctatgcaatgaaggcgctgcaagccgggaatgagagcgatgcgagaaaatttcttgaaagaaaaacatctttggagtcaaagctttctgagctgcaggcggcgaatcaaattgctgcaaccaatgccgcacagatgagaaagatgcatgacaaattggtatctgacattggagagctggaagcacgcaaaaatatgatcaaggcgaagtgggcagtagcgaaaacgcaagaaagaatgaacaaactgggcgcttctgtttcaagtacaagccaatcaatgtctgcattcggccgaatggaggacaaagtaaataaggcacttgatcaggccaatgcaatggctgaactgaacagcgctccgcaggatgatatggctgatctgtctgcaaagtatgacactggcggttcaagccaggttgatgatgaacttgcggcgttaaaagcgaaaatgatgctcgataagtaatacataggaggccgcagctttcggctgcggcgtcattttatcataaaatgtgagagggaacggtaatgataatatcttataagtgtccgaactgcggcagtgatatggcatttgacagtgaaaccggctcgttatcctgcagcagctgcggaagacaggacaatattgaaagccttccgaaagaaaacattgcggca

SEQ ID NO: 82 - protein, 227 aa, Bacillus subtilis
MSIIGRFKDIMSANINALLDKAENPEKMVDQYLRNMNSDLAKVKAETAAVMAEEQRAKREYHECQADMEKMESYAMKALQAGNESDARKFLERKTSLESKLSELQAANQIAATNAAQMRKMHDKLVSDIGELEARKNMIKAKWAVAKTQERMNKLGASVSSTSQSMSAFGRMEDKVNKALDQANAMAELNSAPQDDMADLSAKYDTGGSSQVDDELAALKAKMMLDK

SEQ ID NO: 83 - DNA, 1072 bp, Bacillus licheniformis
  gene (1)..(1072): acoA
tactgccgggttttgtccatctttacgccggagaagaagcggtcgctgcgggcgtctgcgctcatctgaatgatgaagacaccatcacgagcacacatcgcggccatgggcactgcattgcaaaagggtgcgacttgaaaggcatgatggccgagatatacggaaaatctgcaggactttgtaaaggaaaaggcggatcgatgcatattgcagactttgacaaagggatgctaggcgcaaacggaatcgtcggcgggggatttcctttggcctgcggagcagctctcactgcaaagtataaaaagactaaaaatgtaagcgtttgcttttttggcgacggcgccaacaaccaggggacatttcacgaagggatcaaccttgccgccatctggaagctccctgtcatatttgtcgccgaaaacaacgggtatggcgaagcgactccgttttcctatgcgtcaagctgcgagtcgatcgcagatcgtgcggccgcttatgggattccgggcgttcgcgttgatggaaaagatgcggccgcggtcttccaggctgcgggccgggcgatagagcgtgccaaaaacggagaaggaccgacgctcattgagtgtatgacataccggaactacggccattttgaaggagatgcccagcgctacaaaacaaatgaagaaaaagcggtgcatcaggaagaaaaagatgccattgccgtgttcaaaagcgaactgctggcaaaagggctgatgagtgaagaagagcagtcgcggattgaaaaagaagttgaagaagcggtcgaggaagcggtcgcattcagcgaaaaaagcgcttatccggaagaaaccgaactgctgacggatgtatatgtatcctattaattcatgaagggggaaggaaacatgacgagggaaatcagcatgtcagccgcattgaatgaagcgatcaaacttgcgatgagaagggacgatcacgtcattttaatgggggaagatgtcgcaggaggagcgaatgtcgatcatctgcaggatgatgaagcatggggcggtgtgctcggcgtcacgaaagggattgttcagga

SEQ ID NO: 84 - protein, 223 aa, Bacillus licheniformis
MHIADFDKGMLGANGIVGGGFPLACGAALTAKYKKTKNVSVCFFGDGANNQGTFHEGINLAAIWKLPVIFVAENNGYGEATPFSYASSCESIADRAAAYGIPGVRVDGKDAAAVFQAAGRAIERAKNGEGPTLIECMTYRNYGHFEGDAQRYKTNEEKAVHQEEKDAIAVFKSELLAKGLMSEEEQSRIEKEVEEAVEEAVAFSEKSAYPEETELLTDVYVSY

SEQ ID NO: 85 - DNA, 964 bp, Bacillus licheniformis
  gene (1)..(964): ahpC
tatcatttgtgccaatcatactatatgaaaatgaaactccaactcttttctttagctgaaatacttaacaaaaaaataaaggtctttccccttgacaaaaaatatttattatttgataatttgtatataattataataattatagagaaataaagtttacaattttatttctcttaaactacattcaggaggacgatattatgtctctaatcggaaaagaagtacaaccattcgcagcaaaagcttacaaaaacggtgagtttatcgatgttacacacgaagatctgaaaggccaatggagcattttctgcttctatccagcggatttctcatttgtttgcccgactgagcttgaagaccttcaaaaccactacgaagctctaaaagaactaggtgtcgaagtatactctgtttctacagatactcacttcgtacataaaggatggcatgacagctctgagaaaatcggcaaaatcacttacgcgatgatcggcgacccatctcaaacgatctcccgcaattttgaagtgctgaacgaagaagaaggtcttgctgaccgcggaacattcatcatcgatccggacggcgtcatccaagctgttgaaatcaacgcaggcggtatcggccgtgatgcgagcatcctgatcaacaaagtaaaagcggctcaatacgtacgccaaaacccaggcgaagtttgcccggctaaatgggaagaaggttctgaaacactgacaccaagccttgacctagtaggcaaaatctaaggagtgcattcaattggtacttgaagcaaatatcaaagcacaattaaaccaatacatgcagatgattgaaaatgacatcgtccttaaagtcagtgcaggcgatgacagtgtctctaaggatatgctggctcttgtagaagagctggcttccatgtcatcaaaaatcactgttgaaaaggcgcagctcgacagaacgccaa

SEQ ID NO: 86 - protein, 187 aa, Bacillus licheniformis
MSLIGKEVQPFAAKAYKNGEFIDVTHEDLKGQWSIFCFYPADFSFVCPTELEDLQNHYEALKELGVEVYSVSTDTHFVHKGWHDSSEKIGKITYAMIGDPSQTISRNFEVLNEEEGLADRGTFIIDPDGVIQAVEINAGGIGRDASILINKVKAAQYVRQNPGEVCPAKWEEGSETLTPSLDLVGKI

SEQ ID NO: 87 - DNA, 1930 bp, Bacillus licheniformis
  gene (1)..(1930)
  CDS (201)..(1730)
  misc_feature (201)..(203): First codon translated as Met
tccggacggcgtcatccaagctgttgaaatcaacgcaggcggtatcggccgtgatgcgagcatcctgatcaacaaagtaaaagcggctcaatacgtacgccaaaacccaggcgaagtttgcccggctaaatgggaagaaggttctgaaacactgacaccaagccttgacctagtaggcaaaatctaaggagtgcattcaattggtacttgaagcaaatatcaaagcacaattaaaccaatacatgcagatgattgaaaatgacatcgtccttaaagtcagtgcaggcgatgacagtgtctctaaggatatgctggctcttgtagaagagctggcttccatgtcatcaaaaatcactgttgaaaaggcgcagctcgacagaacgccaagcttcagcgtcaatcgtgccgaagaagatacaggcgtgacattcgccggggttccgctcggtcacgaattcacatctttggtgctggcactcttgcaagtaagcggcagaccgccgaaagtcgatcaaaaaatcatcgatcaaatcaagaacatcagcggcgagtaccgcttcgaatcttatatcagcctcacatgccacaactgcccggatgttgtgcaggcattgaacatgatgagcgtgctcaacccgaacatctctcacacaatgatcgacggtgcggcatacaagtcagaagtcgaaagcaaaaacattatggctgtgccgaccgtatacttgaacggcgaatcctttggaagcggccgaatgacgcttgaagaaattcttgcgaagatgggcagcggcacagatgcgtctgagctttctgataaagaaccgttcgatgtactcgttgtcggaggcgggcctgccggcgccagcgcagcaatctatgcggcgcgcaaaggcatccgtacgggaatcgttgccgaacgcttcggcggccaagtccttgacacgatgagcatcgagaacttcatcagcgtgaagcgcaccgaagggcctaagcttgcggcaagccttgaagaacacgtgaaggattatgatatcgacgtcatgaacctgcaacgagcaaaaggcctcgagaaaaaagaccttttccatctagaactcgaaaacggcgctgttctgaagagcaaaaccgtcattctttcaactggcgcacgctggcgcaatatcggcgtacccggcgaacaagagttcaaaaacaaaggtgtcgcatactgccctcactgtgacggaccattgtttgaaggcaaagacgtcgccgtcatcggcggcggcaactccggcatcgaagcggcgattgaccttgcaggcatcgtcaaccatgtgactgttcttgagtttgcgccggagctgaaagcagacaaagttctgcaaaaacgtctctacagcctccccaacgtgactgtcgtgaaaaacgcccaaacaaaagaaatcacgggcgatgaaaacgtaaacggcattacttacgtagaccgtgaaacaggagaagaaaaacacgtcgagctccaaggcgtcttcgtccaaatcggtcttgtgccgaacacggaatggctgcctgaaaccgtcgaacgcaaccgcatcggcgaaatcgcagtcgacaaacgcggcgaaacaaccatccctggattgttcgctgccggcgactgcacagacagcccgtacaaccaaatcatcatttccatgggatccggagcaaacgctgctttaggcgcgtttgactacctgattcggaattaatctataaaaaacgctatgctgaagttttcagcatagcgtttttctgtattcccgcaaccacggctcattcgattttggcaatggaagcatccctcaagcagcgcacctcggcacggaggcgccctctttgtagtcataaccccttataatagaattcattttctcatcttctttaatttggtactataaatatagtagtt

SEQ ID NO: 88 - protein, 509 aa, Bacillus licheniformis
LVLEANIKAQLNQYMQMIENDIVLKVSAGDDSVSKDMLALVEELASMSSKITVEKAQLDRTPSFSVNRAEEDTGVTFAGVPLGHEFTSLVLALLQVSGRPPKVDQKIIDQIKNISGEYRFESYISLTCHNCPDVVQALNMMSVLNPNISHTMIDGAAYKSEVESKNIMAVPTVYLNGESFGSGRMTLEEILAKMGSGTDASELSDKEPFDVLVVGGGPAGASAAIYAARKGIRTGIVAERFGGQVLDTMSIENFISVKRTEGPKLAASLEEHVKDYDIDVMNLQRAKGLEKKDLFHLELENGAVLKSKTVILSTGARWRNIGVPGEQEFKNKGVAYCPHCDGPLFEGKDVAVIGGGNSGIEAAIDLAGIVNHVTVLEFAPELKADKVLQKRLYSLPNVTVVKNAQTKEITGDENVNGITYVDRETGEEKHVELQGVFVQIGLVPNTEWLPETVERNRIGEIAVDKRGETTIPGLFAAGDCTDSPYNQIIISMGSGANAALGAFDYLIRN

SEQ ID NO: 89 - DNA, 3127 bp, Bacillus licheniformis
  gene (1)..(3127): citB
gggtgtattgtataaaaaaatgtatttccagtaaaataaacttttttccgtatataagtcattcttattgaatttaataaaaacattgatatttacttatgtttaaaattgttttaagattaaattgtgagaaaattgtggtgaaatatgccgatttttctggcgtatgatcttgatctttaaagggggattttggagaaatggcgaatcagcaacaaatcgcaaaaaaagacgcttttcaatcaagaaaaacgtttacagtacaaggcaaaacgtacagctattattctttaaaagcattagaggatcaaggaatcggaaacgtatcaaaacttccttattccattaaagttttgctcgaatccgttcttcgtcaagtagacgggagagtcattactgaggaacacgttgaaaacttggcaaaatggggcactgccgaattaaaagatattgacgttcctttcaaaccatcccgcgttattttgcaggactttaccggcgttccagccgttgtagaccttgcttcattaagaaaagcgatggcttcagtcggcggagatccggataaaatcaatcctgaaataccggtagaccttgtcatcgaccactccgtacaagttgacaaagcgggtacggaagatgcattgaccgttaatatggaccttgaattccaacgaaatgcagagcgttacaaatttttaagctgggctaagaaagcgtttaataactatcaagctgttccccctgctacaggtattgttcaccaggtgaaccttgagtatttggcaaacgttgtccatgcagtggaagaagacggcgagattgtcacttatcctgacacgcttgtcggtactgactcccatacaacgatgatcaacggaatcggcgttctcggctggggcgtcggcggaattgaagctgaagcaggcatgctcggccagccttcatatttccctgtacctgaagtaatcggtgcgaagcttgtcggaaagctgccgaacggtacgacagcgacggaccttgcgcttaaagtgacacaggtgctgcgtgaaaaaggcgtagtcggaaagttcgtcgaattcttcggtccgggtgttgcggaactaccgcttgcagaccgtgcgacaatcgcaaatatggctcctgaatacggcgcgacttgcggtttcttccctgttgatgaggaagcattggaatacatgcgtttaacaggccgtgatgaagaacatatcaatgtggtgaaagaatattgccgtcaaaacggactgttctacacgcctgatcaggaagatcctgtatttaccgatatcgttgaaatcgacttgtcaaaagttgaagcgaacctttcaggtccgaagcgtcctcaggatctcattccgctgactgacatgaaagaaacgttccacaagcatttggcaagccctgcgggcaaccagggattcggtttaaacgcgagcgaggctgacaaagaaatcaaatttaagcttgaaaacggcgaagaagccgtcatgaaaacgggtgcgatcgcaattgcggcgattaccagctgtacaaatacatcaaacccttatgtcttaatcggtgcgggcctcgtcgccaaaaaagcggttgaactgggattgaaagtaccgaactatgtgaaaacatctctcgctccgggttcaaaagtcgtaacaggctacttggttaattcaggcctgctgccgtatatgcgcgagcttggatttaacatcgtcggctacggctgtacgacatgtatcgggaactccggaccgcttgcgcctgaaatcgaaaaagcggttgccgaaaacgacctgctgattacgtctgtcctttccggtaaccgtaactttgaaggccggattcatccgcttgtcaaaggaaactacttggcgtctccgcctctagtcgtggcctatgcattggcaggtacggtagatatcgacctgaaaaacgaaccgatcggcgtcggcaaagacggtcaaaatgtgtacttcaacgatatttggccgacgatggacgaaatcaattccgtcgtgaagcaaaccgttactcctgagctgttcagaaaagaatacgagcgcgtgtttgatgacaatgaacgctggaatgcgattgaaacaacggatgaagccctgtataaatgggatgaagagtccacttacattcagaacccgccattctttgaaaacatgtcagttgagccaggtacagttgaaccgcttaaaggcttgcgcattgtcggcaaattcggagactctgtcacgacagaccatatctctcctgcgggagcgatcggcaaagacacgccggccggaaaatatttgcaggaaaaaggcgtttctcctagagacttcaactcatatggttcaagacgcggaaaccatgaagtcatgatgaggggaacattcgccaacatcagaatcaaaaaccagatcgctccgggcactgaaggcggttatacaacgtattggccgaccggagaagtgatgtccatttatgatgcctgcatgaagtataaagaagacggcacaggtctggtcgtcattgccggaaaagactacggaatgggttcttcccgcgactgggcggcaaaaggaacaaatctgctcggcatcaaaaccgtcattgcagaaagctttgaaagaattcacagaagcaaccttgtgctgatgggcgttcttccgctgcaatttaaagagggagaaaacgctgaaacgctcggtttgaccgggaaagaaacaattgaagtagacgtgagtgagtcggtgcgcccgcgcgatctcgtccaagtcaaagcgattgccgaagacggcacggtcaagtcgtttgaagctgttgtccgctttgacagcgaagtggaaatcgattattaccgtcacggcggaatcctgcaaatggtgctccgcaataaaatgaaacagtaattgaatgaggaagagagagcctttagctttctcttcttttttcagcgtaatcctgaatgtggaggatgccgatgatgaaaaaagtgctggctgccgcgtttcttttagtcttggccggtcttgcggtttggaattttacagggcaaaaggaagccgaaatcggcattgagaaaggagacaaggcgcctgattttacgctg

SEQ ID NO: 90 - protein, 908 aa, Bacillus licheniformis
MANQQQIAKKDAFQSRKTFTVQGKTYSYYSLKALEDQGIGNVSKLPYSIKVLLESVLRQVDGRVITEEHVENLAKWGTAELKDIDVPFKPSRVILQDFTGVPAVVDLASLRKAMASVGGDPDKINPEIPVDLVIDHSVQVDKAGTEDALTVNMDLEFQRNAERYKFLSWAKKAFNNYQAVPPATGIVHQVNLEYLANVVHAVEEDGEIVTYPDTLVGTDSHTTMINGIGVLGWGVGGIEAEAGMLGQPSYFPVPEVIGAKLVGKLPNGTTATDLALKVTQVLREKGVVGKFVEFFGPGVAELPLADRATIANMAPEYGATCGFFPVDEEALEYMRLTGRDEEHINVVKEYCRQNGLFYTPDQEDPVFTDIVEIDLSKVEANLSGPKRPQDLIPLTDMKETFHKHLASPAGNQGFGLNASEADKEIKFKLENGEEAVMKTGAIAIAAITSCTNTSNPYVLIGAGLVAKKAVELGLKVPNYVKTSLAPGSKVVTGYLVNSGLLPYMRELGFNIVGYGCTTCIGNSGPLAPEIEKAVAENDLLITSVLSGNRNFEGRIHPLVKGNYLASPPLVVAYALAGTVDIDLKNEPIGVGKDGQNVYFNDIWPTMDEINSVVKQTVTPELFRKEYERVFDDNERWNAIETTDEALYKWDEESTYIQNPPFFENMSVEPGTVEPLKGLRIVGKFGDSVTTDHISPAGAIGKDTPAGKYLQEKGVSPRDFNSYGSRRGNHEVMMRGTFANIRIKNQIAPGTEGGYTTYWPTGEVMSIYDACMKYKEDGTGLVVIAGKDYGMGSSRDWAAKGTNLLGIKTVIAESFERIHRSNLVLMGVLPLQFKEGENAETLGLTGKETIEVDVSESVRPRDLVQVKAIAEDGTVKSFEAVVRFDSEVEIDYYRHGGILQMVLRNKMKQ

SEQ ID NO: 91 - DNA, 2833 bp, Bacillus licheniformis
  gene (1)..(2833): clpC
cttggaattgacctggggataataaaaggtctttccagcaatatacttaatgagctgatgatcctgacacagcctggatttctccagcaatattccggaggagccttaaggccaaacgagagggacatcaggcgggctgctcttatcagggagcgcttgaaattagaaaacaacggcaatagacaggaggatgaaacgatatgatgtttggaagattcacagaaagagcacaaaaagtattggcgcttgctcaagaagaagctctgcgtctgggccataataacattggaacagaacatattctcctcggccttgtccgcgagggagaaggaattgctgcgaaagcgctgcaggcgttaggactcagccctgacaagattcaaaaagaagttgaaagcctcatcggccgcggacaagaaatgtcacaatcgatccattacacaccgagagccaagaaagtgaccgaattggcgatggatgaagcgagaaagcttggccactcctacgttggaactgaacacatcctccttggcttaatccgcgaaggggaaggtgttgcggcaagggtgctgaataatttaggcgtcagcttaaacaaagcgagacagcaagtattgcagctgttgggcagcaatgagagcggaggcgctgcttcaggcgccaacagtagcgccaacacaccaactttagacagtcttgcccgggatctcacggcgattgcaaaagaagacagccttgatcccgtgatcggcagaagcaaagaaattcaacgcgtgattgaagtgttgagccgcagaacgaaaaataaccctgtgttaatcggggagcctggtgtcggtaagactgcgattgccgaaggcctggcccagcagatcatcaataacgaagtaccggagattttacgcgacaagcgcgtcatgacacttgatatgggaacggttgtcgccggcacgaaatatcgcggtgaatttgaagaccgcctgaaaaaagtgatggacgaaattcgtcaagcgggaaatatcatcctcttcatcgatgagctgcatactctcatcggagccggaggagcggaaggggccatcgatgcctccaatatccttaaaccgtcgctggcgcgcggagagctgcaatgcatcggggcgacaacccttgatgaataccgcaaatacattgaaaaagatgctgcactagaacggcgttttcagccgattcaggttgaccagccgtctcctgaagaaagcattcaaatattgaaagggctcagagaccgctatgaagcccatcaccgcgtatcgattacggatgaagcgatcgaagcggcagtaaagctgtcagaccgctacatttcggaccgttttctgcctgacaaggcgatcgacttaatcgatgaagccggctcaaaggttagactgcgttcattcacaacgcctcctaacttaaaagagcttgaacaaaagcttgatgaggtaaggaaagaaaaagatgccgctgttcaaagccaggaattcgaaaaagcagcgtctttacgtgatacggagcagcgcttgcgtgaacaggtcgaagaaaccaagaaaacctggaaagaaaagcaaggtcaagaaaattccgaagtgaccgtcgacgatattgcaatggttgtatcaagctggaccggagtgccggtttccaaaattgcgcagaccgaaacagacaaattgctgaacatggagagcattcttcattcccgcgttatcggccaggatgaggctgtcgttgcagttgcaaaagcagtcagacgcgcgcgtgccggcttaaaagatccaaaacgtcctatcggttcgtttattttccttggcccaacaggcgtagggaaaacggagcttgcccgtgcacttgccgagtccattttcggcgatgaagaagcaatgattcgcatcgatatgtccgagtacatggaaaaacattcaacatcaagactcgtcggttcacctcctggatatgtcggctatgatgaaggcggacagctcactgaaaaagtgagaagaaaaccgtattccgtcgtcttgcttgatgaaatcgagaaagctcacccagatgtcttcaatattttgctgcaggtgcttgaagacggccgcctgactgattcaaaagggcgcacggttgactttaggaatacgattttgatcatgacatcaaacgtcggtgcgagcgaactgagaagaaataaatacgtcgggtttaatgtccaggatgaaacccagaattataaagatatgaaagacaaagtcatgggtgaactaaaacgcgcctttagacctgaatttatcaaccggattgatgagatcatcgtcttccactctttggagaaaaaacatttgaaagatattgtatccctcatgtcagaccaattgaccaagcgtctgaaagaacaggatctttcaattgagctgactgaagcggctaaagaaaaaatcgccgatgaaggcgtggatctcgaatacggagcgcgtcctctcagaagagcaatccaaaaacacgtggaagaccggctgtcagaagagctcctaaaaggccatattcaaaaaggccagcatattgttttggatgtagaagacggagaatttgtcgtaaaaaccgaagctaaaacgaactaatatagagacaaaagaaagaggcatacgatgttcatcagtatgcctcactttcttataaagggagcgttttgaaaaatcaatggctaaaacaaagactaaattcatttgtcactcatgcggctacgaatccgccaagtggatggggaaatgccccggatgcggtgcatggaataccatggtggaagaaacgataaaaaaac

SEQ ID NO: 92 - protein, 810 aa, Bacillus licheniformis
MMFGRFTERAQKVLALAQEEALRLGHNNIGTEHILLGLVREGEGIAAKALQALGLSPDKIQKEVESLIGRGQEMSQSIHYTPRAKKVTELAMDEARKLGHSYVGTEHILLGLIREGEGVAARVLNNLGVSLNKARQQVLQLLGSNESGGAASGANSSANTPTLDSLARDLTAIAKEDSLDPVIGRSKEIQRVIEVLSRRTKNNPVLIGEPGVGKTAIAEGLAQQIINNEVPEILRDKRVMTLDMGTVVAGTKYRGEFEDRLKKVMDEIRQAGNIILFIDELHTLIGAGGAEGAIDASNILKPSLARGELQCIGATTLDEYRKYIEKDAALERRFQPIQVDQPSPEESIQILKGLRDRYEAHHRVSITDEAIEAAVKLSDRYISDRFLPDKAIDLIDEAGSKVRLRSFTTPPNLKELEQKLDEVRKEKDAAVQSQEFEKAASLRDTEQRLREQVEETKKTWKEKQGQENSEVTVDDIAMVVSSWTGVPVSKIAQTETDKLLNMESILHSRVIGQDEAVVAVAKAVRRARAGLKDPKRPIGSFIFLGPTGVGKTELARALAESIFGDEEAMIRIDMSEYMEKHSTSRLVGSPPGYVGYDEGGQLTEKVRRKPYSVVLLDEIEKAHPDVFNILLQVLEDGRLTDSKGRTVDFRNTILIMTSNVGASELRRNKYVGFNVQDETQNYKDMKDKVMGELKRAFRPEFINRIDEIIVFHSLEKKHLKDIVSLMSDQLTKRLKEQDLSIELTEAAKEKIADEGVDLEYGARPLRRAIQKHVEDRLSEELLKGHIQKGQHIVLDVEDGEFVVKTEAKTN

SEQ ID NO: 93 - DNA, 749 bp, Bacillus licheniformis
  misc_feature: 5′end incomplete.  Comparison with clpP from B. subtilis teaches that probably 48 nucleotides are missing, which code for 16 amino acids.
gcgtatgacatttactcacgcttgttgaaagaccgtatcattatgctgggatcagcaatcgatgacaatgttgcgaactccatcgtatctcagcttctattccttgaagccgaagatccagaaaaagatatcagcatctatatcaacagccctggcggctccatcacagccggtatggcgatttatgacaccatgcagtttattaaaccgcaagtctccacgatttgtacgggaatggcggcatccatgggtgcgttccttcttgcagccggcgagaaagggaagcgctacgctcttccaaacagtgaagtcatgatccaccagccgcttggcggagcgcagggtcaggcgactgaaattgaaattgccgcgaaacgcattctttcattgcgcgacaagctgaacaaaatccttgcagagcgcactgggcagccgcttgaagtaatcgagcgcgatacagaccgcgacaacttcaaaacggctgaagaagcaaaagaatacggcctgattgataaagtgcttacacgcaatatcgacgcacagaagtaaacagccaaaaagctgccggagtcaccggcagcttttttttactccaaatgggttgtcccattcgctatatgtaaagcttctataaatccttccactttttctcgaggacagggtagcgataatcattcatgttcccgatcaactccgcagggcttgccgaagcatgaatcagctcacggtgcgatttgttcaaaaagctt

SEQ ID NO: 94 - protein, 182 aa, Bacillus licheniformis
AYDIYSRLLKDRIIMLGSAIDDNVANSIVSQLLFLEAEDPEKDISIYINSPGGSITAGMAIYDTMQFIKPQVSTICTGMAASMGAFLLAAGEKGKRYALPNSEVMIHQPLGGAQGQATEIEIAAKRILSLRDKLNKILAERTGQPLEVIERDTDRDNFKTAEEAKEYGLIDKVLTRNIDAQK

SEQ ID NO: 95 - DNA, 1180 bp, Bacillus licheniformis
  gene (1)..(1180): codY
ggggcgagaaggcttcacaccattttggaacgtcttttggaagaactgtcattcgaggctccggatgtgacgatggaagaagtggtcatcacgcctcaatatgttgaagaaaagctcggttcgatcgctaagaataaggatttaagccagtttattttataaacaattaaataagagtaatttaggaggatcaatttattatggctttattacaaaaaacaagaaagattaatgcaatgctgcaaaatgcggcagggaagccggttaattttaaagaaatggccgaaacgcttcgcgatgtgattgattcgaacattttcgtcgtcagccgcagaggaaagcttctcggtttctcgatcaaccagcagatcgaaaatgaccggatgaaaaaaatgcttgaagagcgccagtttcctgaagattatacaaaaagcctgttcaacattccggaaacgtcatctaaccttgacatcaacagcgaatacacagctttccctgtggaaaaccgcgatttgttccaagcgggtctgaccacggtcgttccgatcatcggaggaggcgaaagactgggcaccttgattctgtcccgccttcaggagaaattcgaggatgacgatctgatccttgccgaatatggcgccactgtcgtaggaatggaaattctcagagagaaagcggaagagatcgaagaggaagcgcgcagcaaagcggtcgttcaaatggcgatcagttcactatcttacagcgagcttgaagcaattgaacatatttttgaggagcttgacggaaatgaaggcctgcttgtcgctagtaaaattgcagaccgcgtcggcatcacccgctcggtcattgtcaacgcgctccgtaaacttgaaagcgccggtgtcattgaatcaaggtcactagggatgaaaggcacatacatcaaagtgctgaacaacaaatttttgatggagttagaaaaactaaagtctcattaaaaggcgaaagccgcagaacatgccgttctgcggctttttttatttcatgttttaaactatattatcgtaattttttcatatcaaaaaacaactaaaacgcggtgcaaaatcaatgcgttacttgaataaaagatatactaacataattttttgaaactttaatgaacgtaacattacatagttcttataatctaggtcaa

SEQ ID NO: 96 - protein, 259 aa, Bacillus licheniformis
MALLQKTRKINAMLQNAAGKPVNFKEMAETLRDVIDSNIFVVSRRGKLLGFSINQQIENDRMKKMLEERQFPEDYTKSLFNIPETSSNLDINSEYTAFPVENRDLFQAGLTTVVPIIGGGERLGTLILSRLQEKFEDDDLILAEYGATVVGMEILREKAEEIEEEARSKAVVQMAISSLSYSELEAIEHIFEELDGNEGLLVASKIADRVGITRSVIVNALRKLESAGVIESRSLGMKGTYIKVLNNKFLMELEKLKSH

SEQ ID NO: 97 - DNA, 601 bp, Bacillus licheniformis
  gene (1)..(601): cspB
accctgagcattaaatttcactttttttaaaaaaattgtgtttagcagttgtaatttcgtttaaacactggtaaagtaaaggtaattatttttgttcgaactatctttaagaagaaagttttgtaagagttttcgtcttggaagtttgttttagagcaagaatagtgaatttaagcgttatcgctttaggaggaaatttcatgctagaaggtaaagtaaaatggtttaactctgaaaaaggtttcggattcatcgaagttgaaggacaagacgatgtattcgttcatttctgtgctattcaaggcgaaggcttcaaaactttggaagaaggccaatctgtatctttcgaaatcgttgaaggaaaccgcggaccacaagctgctaacgtaacaaaagcgtaagctgtgaaccgctgatcttcatacaggactgctgaccgaatgtcagcagtctttttatatttaaaaaagcctgacgcccggaaaccggtgtcaggctttttttaaacgtataccgcttatgaagcgatgtctctctttgtgaatacaacaaaggcgagcacatgaaaaacgataaaatagacagccagcatcacaatcga

SEQ ID NO: 98 - protein, 66 aa, Bacillus licheniformis
MLEGKVKWFNSEKGFGFIEVEGQDDVFVHFCAIQGEGFKTLEEGQSVSFEIVEGNRGPQAANVTKA

SEQ ID NO: 99 - DNA, 1429 bp, Bacillus licheniformis
  gene (1)..(1429): des
gtctaaaaaaaaacaattcccatagggaagcagggagagagtttggcccgataaagagccgggtataattttcataaaattgagctggaaatcagtttcgagtttccaataaagagggctaagaacgatacttatatgatgtatgacgcttggactaacgtaaacttagaaactcatcagggaaggggaaacttttaaaaatgaatgaccaaaatctaaagactttgagaaaactagttacaccttatgaaaagtccgatttacaaaaaagcatttaccaaatcattaacacattgataccatttttcctgttatggtgtttagcatataagagcttgtcgatttcttatttccttacattagcgatttctatcgttgcggcaggttttttagtgagaaccttcatcatctttcatgattgctgccactattccttttttaagaacaaaaaggcgaatcgaatccttggaacaatcacaggaattctaactttgcatccttttgatcattggggacgcgaccattctatccaccatgcgacaagcagcaacctggacaaacgaggtacaggcgatatttggttgctgaccgttgaagaatataaggaagcctcaactaagacaaaaataatgtaccgtttatacagaaatccgtttgttatgtttatgattgggccgatttacgtcttcgggattaccaatcgttttaatcgtaaaggggcaaaacgcaaagaacggatgaatacgtatgtaacgaacttgggaatcgccgctttgacagcacttttatgctgggctattggctggcaaaacttcctgctggttcaggcgccaatttttatgatatcgggatctctcggaatttggatgttttatattcagcatacgtttgaggattcttattttgaagaagatgagcattgggaatatgtaaaagcagcagttgaaggaagctctttttataagcttccaaaagtcatgcaatggctaacaggcaatatcggtttccatcatgttcatcatttaagcccgagagtcccaaactataagcttgaagaagtgcataacaacattgaaccattgcaaaacgttccaaccattacactggcaacaagtcttaagtcgttaaagttccgactatgggatgaagaaagcaaaaaatttgttggttttagccacttaaaaaaagcttctaaaagccaagtatcagcgcaactaagaacggattaaataaacttcccataatcgatgataataggcagtgtatcttgagaataaaagtgcagactcgcacaacggttttgtgcagaggtcagcacttttttagtgccaaatatacttgcagacttggacaatgctgtacattttttgccgattttccgcaaaaaaataataccaaaggcaattaaaactttggtattatttggtgt

SEQ ID NO: 100 - protein, 342 aa, Bacillus licheniformis
MNDQNLKTLRKLVTPYEKSDLQKSIYQIINTLIPFFLLWCLAYKSLSISYFLTLAISIVAAGFLVRTFIIFHDCCHYSFFKNKKANRILGTITGILTLHPFDHWGRDHSIHHATSSNLDKRGTGDIWLLTVEEYKEASTKTKIMYRLYRNPFVMFMIGPIYVFGITNRFNRKGAKRKERMNTYVTNLGIAALTALLCWAIGWQNFLLVQAPIFMISGSLGIWMFYIQHTFEDSYFEEDEHWEYVKAAVEGSSFYKLPKVMQWLTGNIGFHHVHHLSPRVPNYKLEEVHNNIEPLQNVPTITLATSLKSLKFRLWDEESKKFVGFSHLKKASKSQVSAQLRTD

SEQ ID NO: 101 - DNA, 1839 bp, Bacillus licheniformis
  CDS (1)..(1839)
  gene (1)..(1839): dnaK
atgagcaaagtaatcggaatcgacttgggaacaacgaactcatgcgttgcagtgcttgaaggcggagagccgaaagtcatcccaaatccagaaggagcccgcacgactccttcagttgtcgcatttaaaaacggagaacgccaggtgggtgaagtggcgaaacgccagtccatcacgaacccgaacacgatcatgtcgatcaagcgccacatgggcactgactatacagttgaaattgaaggcaaaaagtacacgccgcaagaagtgtctgccatcatccttcagcatttgaaatcatatgctgaaagctatttgggcgaaaccgtgtctaaagccgtgatcacagttcctgcatacttcaacgatgcagagcgtcaagcgactaaagatgccggtaaaatcgccggacttgaagttgagcggatcatcaacgagccgactgcagctgcgcttgcctacggtctggataaaacggaagaagatcagacgatcctcgtctacgaccttggcggcggtacattcgacgtttccatccttgaattgggagacggcgtattcgaagttcgctctacagcgggtgacaaccgccttggaggagacgactttgaccaagtgatcatcgatcatctcgtctcagaattcaaaaaagaaaacggcatcgacctttctaaagacaaaatggcgcttcagcgcttaaaagatgctgctgagaaagcgaaaaaagacctttccggcgtatcttctacgcaaatttccctgccgtttatcacagcgggtgaagcaggtccgctgcaccttgaattgacactgacccgcgcgaagtttgaagagctgtcttcacacttggtagagcgcacaatgggtcctgtacgccaggctcttcaagatgcgggactttctgcaagcgaaatcgacaaagtcatccttgtcggcggatcaacacgtattcctgccgttcaggaagcgatcaaaaaagaaactggaaaagaagcgcataaaggcgtcaaccctgacgaagtcgtagcccttggtgcagcaattcaaggcggcgtcatcacaggcgatgtcaaagatgttgtccttctggacgtaactccgctgtcacttggtatcgaaacgatgggcggcgtgttcacgaagctgattgagcgcaacacgacaatcccgacaagcaagtcccaagtgttctcaacagcggccgacaaccaaacagcggttgacatccacgtgctccaaggtgagcgcccaatggctgccgacaacaaaacgctcggacgcttccagcttacagatatcccgccggctcctcgcggcgtaccgcaaatcgaagtgtcatttgacattgataaaaacggtatcgtcaacgtcagcgcgaaagatctgggcactggcaaagagcaaaacatcacgatcaaatcttcttcaggcctttctgacgacnagatcgagcgtatggtgaaagaggctgaagaaaacgccnaancanatgcgaagaanaaagaagaaatcgagcttcgcaacgaancggaccagcttgtcttcacaactgaaaaaacgcttaaagatcttgaaagccaagccgatgaagcacaggtgaaaaaagcgaatgaagcaaaagacgcgctaaaagctgcgatcgagaaaaatgaccttgatgaaatcaaggcgaanaaagatgagctgcaagcgatcgttcaagagctttcaatgaagctttacgaagaagccgcaaaacaggctcaggctcagcaagacggcggaactggcgctaaaaaagccgatgataatgtcgtggacgctgaatatgaagaagtaaacgacgacaaagatcaaaaataa

SEQ ID NO: 102 - protein, 612 aa, Bacillus licheniformis
  misc_feature (481)..(481): The ′Xaa′ at location 481 stands for Lys, Glu, or Gln.
MSKVIGIDLGTTNSCVAVLEGGEPKVIPNPEGARTTPSVVAFKNGERQVGEVAKRQSITNPNTIMSIKRHMGTDYTVEIEGKKYTPQEVSAIILQHLKSYAESYLGETVSKAVITVPAYFNDAERQATKDAGKIAGLEVERIINEPTAAALAYGLDKTEEDQTILVYDLGGGTFDVSILELGDGVFEVRSTAGDNRLGGDDFDQVIIDHLVSEFKKENGIDLSKDKMALQRLKDAAEKAKKDLSGVSSTQISLPFITAGEAGPLHLELTLTRAKFEELSSHLVERTMGPVRQALQDAGLSASEIDKVILVGGSTRIPAVQEAIKKETGKEAHKGVNPDEVVALGAAIQGGVITGDVKDVVLLDVTPLSLGIETMGGVFTKLIERNTTIPTSKSQVFSTAADNQTAVDIHVLQGERPMAADNKTLGRFQLTDIPPAPRGVPQIEVSFDIDKNGIVNVSAKDLGTGKEQNITIKSSSGLSDDXIERMVKEAEENAXXXAKXKEEIELRNEXDQLVFTTEKTLKDLESQADEAQVKKANEAKDALKAAIEKNDLDEIKAXKDELQAIVQELSMKLYEEAAKQAQAQQDGGTGAKKADDNVVDAEYEEVNDDKDQK

SEQ ID NO: 103 - DNA, 1693 bp, Bacillus licheniformis
  gene (1)..(1693): eno
cgaagaaggcaagccgcacaccgcacatacgacaaacccggttcctgtcatcgtcacaaaagaaggcgtgacattaagagaaggcggaatcctcggggatctggcgccaactcttttagacttgctgggtgttgaaaaaccgaaagaaatgacaggatcgtcattaattcaaaaataagcattaaaggagagaaacaaacatgccatacattgttgatgtttatgcacgtgaagtattagactcccgcggtaacccgacggttgaagttgaagtatacactgaatcaggagcttttggacgcgcgctggttccaagcggagcttccactggcgaatacgaagctgttgagcttcgcgacggcgacaaagaccgctaccttggaaaaggcgttttaacagccgttaacaacgtgaacgaaatcatcgcacccgagcttatcggctttgatgtgactgagcaagtatcaatcgacaaattgctgatcgaacttgacggaactgaaaataaaggcaagctcggcgccaacgccatccttggtgtatcaatggccgttgcccgcgcagctgcggatttcttgcagattcctctataccaataccttggaggattcaactctaaaacgcttcctgtaccgatgatgaacatcgtaaacggcggagagcatgcggacaacaacgttgacattcaagaattcatgatcatgcctgtcggtgcggaaaacttccgcgaagcacttcgcatgggagcacaaattttccacagcctgaaatcagtcttgaaagaaaaaggcttgaacacagctgtaggtgatgaaggcggattcgctccaaaccttggatctaacgaagaagcgcttcaaacaatcgttgaagcgatcgaaaaagcaggattcaaacctggcgaagaagtgaaattggcaatggatgctgcatcttctgagttctacaacaaagaagacggcaaataccatcttgcaggcgaaggcgttgtaaaaacgtcagctgaaatggttgactggtacgaggagctgacttctaagtacccaatcatctcaatcgaagacggccttgacgaaaacgactgggaaggccacaaacttctgactgagcgtcttggctcaaaagttcagcttgtcggtgacgaccttttcgtaacaaacacgaaaaagcttgctgaaggaatcaaaaacggcgtcggcaactctatcctgatcaaagtaaaccaaatcggtacattgactgaaactttcgatgcgatcgaaatggcgaaacgcgcaggctacactgccgttatctctcaccgctccggtgaaactgaagacagcacaatcgctgacatcgctgtggcaacaaacgcaggacaaatcaaaacaggtgctccgtctcgtacggaccgtgttgcgaaatacaaccagcttcttcgcatcgaagatcaattggctgaaactgcgcaataccacggtattcaatctttctacaacttgaataagtaagcataaaaaagaggttggctgcagatgcggccaacctcttttaaatgtgaaagctgaatttcctttaccctgacaaaagctgtcagggtttttgattataataggctgtatcaattgcgattggagtgttactggatgcagtcgaaaacagtatatctttatgcattccatacaatgtcagattgggaatatggatattt

SEQ ID NO: 104 - protein, 430 aa, Bacillus licheniformis
MPYIVDVYAREVLDSRGNPTVEVEVYTESGAFGRALVPSGASTGEYEAVELRDGDKDRYLGKGVLTAVNNVNEIIAPELIGFDVTEQVSIDKLLIELDGTENKGKLGANAILGVSMAVARAAADFLQIPLYQYLGGFNSKTLPVPMMNIVNGGEHADNNVDIQEFMIMPVGAENFREALRMGAQIFHSLKSVLKEKGLNTAVGDEGGFAPNLGSNEEALQTIVEAIEKAGFKPGEEVKLAMDAASSEFYNKEDGKYHLAGEGVVKTSAEMVDWYEELTSKYPIISIEDGLDENDWEGHKLLTERLGSKVQLVGDDLFVTNTKKLAEGIKNGVGNSILIKVNQIGTLTETFDAIEMAKRAGYTAVISHRSGETEDSTIADIAVATNAGQIKTGAPSRTDRVAKYNQLLRIEDQLAETAQYHGIQSFYNLNK

SEQ ID NO: 105 - DNA, 808 bp, Bacillus licheniformis
  gene (1)..(808): glnR
cttatgtggcgtatgtccaaggcggattaacctactcccatgtgaaaaatgcgatatgcagtgctgtggatgcgctcatgaagcagggattaatgtaaagaaaataaagaatattttcatgtaaagaatccttacatcgagttgacacattatataacatcacatataataaatttatataagaaaaggaggaaattgagatgagtgataaaattcgccgctcaatgcccttatttccgataggtatcgtcatgcagctgactgaactgtccgcaaggcaaattcggtattatgaggaaaatggactggtatttcctgcaagaagcgaagggaacagacgtttattttcattccatgatgtagataagctgctcgaaattaaagatttgatcgaacaaggtgtaaacatggcggggatcaaacaaattttcgccaaaacagaaaacgaacaaaaagaagacaaacaagacaagccgaaaaaagcagagaagcacaacttgtcggatgaagaactgagaaaactgctgaaaaacgagctcatccaagcaggccgtttccaaagagggacgacattccgccagggagacatgtccaggttttttcattaatttgttagcaactgcattgctatatacatttaccttttagaggaggagttttacgaatggcaaagtatacaagagacgacattgttaaactagtaaatgaggagaatgtaaaatatattcgccttcagtttacagacattcttggaacgattaaaaacgttgaaattcctgtgagccagctggaaaaagcacttgataat

SEQ ID NO: 106 - protein, 135 aa, Bacillus licheniformis
MSDKIRRSMPLFPIGIVMQLTELSARQIRYYEENGLVFPARSEGNRRLFSFHDVDKLLEIKDLIEQGVNMAGIKQIFAKTENEQKEDKQDKPKKAEKHNLSDEELRKLLKNELIQAGRFQRGTTFRQGDMSRFFH

SEQ ID NO: 107 - DNA, 2035 bp, Bacillus licheniformis
  gene (1)..(2035): groEL
tcaggccgtgtgttagagagcggagagcgcgttgcattagaagttaaaacaggcgaccgcatcatcttctcaaaatatgcaggtaccgaagtgaaatatgaaggtactgactacttaatcttgcgtgaaagcgacattttagctgttatcggctaagtcataaataaaaacgagacaattatataggaggttcgaataacatggcaaaagatattaagtttagcgaagaagcccgccgttcaatgctgcgcggtgtagatgcattggcggatgctgtaaaggtaactttgggacctaaaggacgcaacgtcgttcttgagaaaaaattcggttctccattaatcacaaacgacggtgtaaccatcgcgaaagaaatcgagcttgaagacgcgttcgaaaacatgggcgcaaagcttgttgctgaagttgcgagcaaaacaaacgatgttgccggtgacggtacaacaacagcgacagttctagctcaggcgatgattcgcgaaggtcttaaaaacgtaactgccggcgctaaccctgtaggcgtgcgcaaaggtatcgagcaggctgtggctgtagctgttgaaagcctgaaagaaatctctaaaccaattgaaggcaaagaatcaatcgcacaagttgcttcaatctccgctgcagacgaagaagtcggaagcctgatcgctgaagcaatggagcgcgtcggcaacgacggtgttatcacgatcgaagaatccaaaggattcacaacagagcttgaagtggttgaaggtatgcagttcgaccgcggatatgcgtctccttacatggtgacggattccgataagatggaagcggttcttgagaatccgtacatcttagtaacagacaaaaaaatcacaaacattcaagaaatcctgccggtgcttgagcaagtcgtgcaacaaggcaaaccgttgcttctgattgctgaagacgttgaaggtgaagctcttgcaacattggttgtcaacaagcttcgcggaacattcaacgcagtggctgttaaagcgcctggattcggcgaccgccgcaaagcgatgctcgaagacatctctatcctgacaggcgccgaagtgatcacagaagatttaggtcttgaccttaaatcaactcaaatcaatcaattgggacgcgcttctaaagttgttgttacaaaagaaaacacaacaattgttgaaggtgcgggagatacagagcaaatcgcggcacgcgtcaaccaaatccgcgctcaagttgaagaaacaacttctgagttcgataaagaaaaattacaagaacgccttgctaagcttgcaggcggcgtagctgtcatcaaagtcggagctgcgactgaaacagaattgaaagaacgcaagcttcgcatcgaagacgccctgaactctactcgcgctgcggtagaagaaggaatcgtatccggcggtggtacagctcttgtcaatgtttacaataaagtagctgcccttgaagctgaaggcgacgaactgactggtatcaacatcgttcttcgcgctcttgaagaaccaatccgtcaaatcgcgcacaacgcgggtcttgaaggatctgtcatcgttgagcgcctgaaaaatgaagaaatcggcgtcggctacaacgctgcaacaggcgaatgggtgaacatgatcgacaaaggtatcgttgaccctacaaaagtaacacgctcagctctgcaaaacgcagcatctgtagcggctatgttcctgacaactgaagcagttgttgccgacaaaccggaagaaaacaaaggcggcgcaggaatgcctgacatgggcggcatgggcggcatgggcggcatgatgtaagcccagcgtccaaacgctgatatatcaaggaaaagaggctctttttagagcctctttttttaaaaaatcacataaaaatcacattttttcggttttaaaacttgtgataccttgtaggaaacatttcattttattaaatgtttctttcttcatcttgtttgttatgtgagtataaatttcaattgtggctctcatatcag

SEQ ID NO: 108 - protein, 544 aa, Bacillus licheniformis
MAKDIKFSEEARRSMLRGVDALADAVKVTLGPKGRNVVLEKKFGSPLITNDGVTIAKEIELEDAFENMGAKLVAEVASKTNDVAGDGTTTATVLAQAMIREGLKNVTAGANPVGVRKGIEQAVAVAVESLKEISKPIEGKESIAQVASISAADEEVGSLIAEAMERVGNDGVITIEESKGFTTELEVVEGMQFDRGYASPYMVTDSDKMEAVLENPYILVTDKKITNIQEILPVLEQVVQQGKPLLLIAEDVEGEALATLVVNKLRGTFNAVAVKAPGFGDRRKAMLEDISILTGAEVITEDLGLDLKSTQINQLGRASKVVVTKENTTIVEGAGDTEQIAARVNQIRAQVEETTSEFDKEKLQERLAKLAGGVAVIKVGAATETELKERKLRIEDALNSTRAAVEEGIVSGGGTALVNVYNKVAALEAEGDELTGINIVLRALEEPIRQIAHNAGLEGSVIVERLKNEEIGVGYNAATGEWVNMIDKGIVDPTKVTRSALQNAASVAAMFLTTEAVVADKPEENKGGAGMPDMGGMGGMGGMM

SEQ ID NO: 109 - DNA, 1861 bp, Bacillus licheniformis
  gene (1)..(1861): katA
tttgaatgctgatccattcttcttccccctttatttggaattaacaataatttatcccaatccagaaaagttattcagcatttatcatgatcaaatcaacgataataaatttttctttataataattataaataaatattgtttttttcttgagaaatgttatcattgttttgtaattaaaatttacgcgaggtgatcctttgatgacgacaaataaaaacaacctaacaaccagctggggcgctccagttggagacaaccaaaactcaatgacagctggttcacgcggaccgacgctgattcaggacgtccacttgctcgaaaagctcgcccacttcaatagagagcgtgtgcctgaacgggttgtgcatgcaaaaggcgccggtgcgcacggttattttgaagtcacaaatgacgtttccaaatatacaaaagccaaatttttatcagaagtcggaaaacgcacgccgttatttgtccgtttttcaactgttgccggcgaaaacggttcagccgattccgtccgcgacccgcgcggtttcgctgttaaattttatacagaggaagggaactatgatctcgttggaaacaatacacctgttttctttatccgcgatgcgattaaattccctgacttcattcatacacaaaagcgccatccggtcacacatttgaaaaacccggatgccgtatgggatttttggtcattgtctcctgaatccctgcaccaggtaaccatcctgatgtctgacaggggcatccctgcgacttaccggcacatgcacggcttcggctcccacacattcaaatgggtgaatgccgaaggagaaggcgtctggatcaaataccacttcaaaacagaacaaggcattaaaaacctgaccgaagaagtggggacaaagattgcaggtgaaaaccccgattatcacacacaggacttgtatgaggcgattgaaaaaggcgacttcccggcatggaagctgtacgttcaaataatgccgctggaggatgcggatacgtaccgttttgatccgttcgatgtgacaaaagtatggtcgcaaaaagattatccgctgattgaagtcggccgcatggttctaaaccggaatccggaaaactatttcgcagaggttgaacaagctacattctcccctggaacattggtgcccggcattgaaccgtctcccgacaaaatgcttcagggacgcctgttcgcatatgcggatgcacaccgctaccgcgtcggcgcaaaccataacagcctgccgatcaaccgtccgaaagcggaagttcataattatcaaagagacggacaaatgcgctttgattcaaacggcggcggctctgtctactatgagccgaacagcttcggaggccctcaggaaactccggaaaacaaaacaacggcttatccggtgtcaggttcggcggacagcacggcctacgaccataacgaccactatacacaggccggagatctttaccgcctcttgagcgaagaagagcggacccgccttgtcagcaatatcgtaggttcgatgaagcaggtgacaaaagacgaaattaagcttcgtcaaattcagcatttctataaagcagatccggagtacggcacacgagttgccgaaggattgggcttgtctgttccgcaggaagtttaaggctaatatcatttcaatcattctcaatgaaattctcattttctcaggtacaggcacccgtcctcttaagcatgcgggtgcctgcccttaatcaaagaacgacaggggatgttacgtgtggaagacgagcagctcgtccctttattaaaaaagcggggtttacgaattacagctcagcgcgtcctgattttaaaaacgat

SEQ ID NO: 110 - protein, 486 aa, Bacillus licheniformis
LMTTNKNNLTTSWGAPVGDNQNSMTAGSRGPTLIQDVHLLEKLAHFNRERVPERVVHAKGAGAHGYFEVTNDVSKYTKAKFLSEVGKRTPLFVRFSTVAGENGSADSVRDPRGFAVKFYTEEGNYDLVGNNTPVFFIRDAIKFPDFIHTQKRHPVTHLKNPDAVWDFWSLSPESLHQVTILMSDRGIPATYRHMHGFGSHTFKWVNAEGEGVWIKYHFKTEQGIKNLTEEVGTKIAGENPDYHTQDLYEAIEKGDFPAWKLYVQIMPLEDADTYRFDPFDVTKVWSQKDYPLIEVGRMVLNRNPENYFAEVEQATFSPGTLVPGIEPSPDKMLQGRLFAYADAHRYRVGANHNSLPINRPKAEVHNYQRDGQMRFDSNGGGSVYYEPNSFGGPQETPENKTTAYPVSGSADSTAYDHNDHYTQAGDLYRLLSEEERTRLVSNIVGSMKQVTKDEIKLRQIQHFYKADPEYGTRVAEGLGLSVPQEV

SEQ ID NO: 111 - DNA, 1861 bp, Bacillus licheniformis
  gene (1)..(1861): katE
atacagaagaagggaactatgatcttgtcggcaacaacatccccgttttcttcattcaggatgccatcaagttccctgacctggtgcacgcctttaaaccggaaccgaacaacgaaatgccgcaggcttctacggcgcacgatacgttttgggactttgtcgccaacaatgaagaaacggcccacatgatcatgtgagcaatgtctgacagggcgattccgcgaagctacaggatgatggaaggcttcggcgttcatacgttcaggtttgtaaacgaagaaggcaaggcgcactttgttaaatttcactggaagcccgtcctcggcgtccattcgctcgtctgggacgaagcgcagacaatcgccggaaaagatcccgactatcaccgccgcgatctgtgggaagcgatcgaaagaggcgatgaagtggagtatgagctcggcgtgcaaatgattgacgaagaagacgagttcaagtttgacttcgatatccttgacccgacaaagctttggccggaagaaatcgtccctgtcaaaatcatcggcaaaatgacgctgaatcgaaaccaggataacgtttttgcggaaaccgaacaggccgctttccatccgggccatgttgtgccggggatcgatttcacaaacgatccgctattgcaaggccggctcttttcttatacggacacacagctgatccgattgggaggtccgaactttcacgaaattccaatcaaccgcccggtctgtccgttccataacaaccagtatgacggctatcacaggatgacgattaacaaagggcctgtcgcttatcataaaaattcgcttcaaaacaacgatccggcgcctgcttcagaagaagaaggcggatatgtgcactacgaggaaaaagtcgagggcaaaaaaatccgccagcgcagcgaaagctttaacgaccactattcgcaagcgaagctgttttggaacagcatgtcccccgttgaaaaagaacatatcatttcggcgttccgctttgaagtcggaaaggtcaaaagcaaggacgtccgccgccaagtcgttcatatgtttaaccgggttgacggggagcttgccaagcaaatcgctgcgggtgtaggcgtagagccccctgaaaaagacgaaggctccaacgtgacgttcaaatcccctgcgctcagccaggagaatacggtgaaactgccgcagacaaggacagtcgccattttagcagaacaaggttttgatgatgaagacttgagccgtgtgttgaaagaattcaaaaaagccggcatcattccggacatcgtcagctcggcactcggagtaatcgaaggggccggcggcacagaaattgaagtcggaaacactttgcagacggtcgattctgtgctgtacgatgccgtttatattccgggcggccaagaaagcatagaacgcctgaaattgcataaagccgcatcggattttgtcaatgaagcattcggacattataaagcggtcggagccgcaggcgaaggggtcgatctcctgctgtccgccgccggggcccacgcggcttcacagccgggcattatcacctctcgtggtgataagaacaaagacgacttcggcaaaaagctggtcgaagcgatcagcgggcaccgccattgggataggcaagtttaataataaagcaggcaggctagatgcctgcccttttaatctatttttctaccaaacaaacagcccgacaatccccgcgctcaacagcgacactgctattccgctgacgaggagcttccacacatttcttccgataatcgccgatttttctccgttaaataaggaattgtacgttccatagatcatcccgatcgtactgaagt

SEQ ID NO: 112 - protein, 486 aa, Bacillus licheniformis
MSDRAIPRSYRMMEGFGVHTFRFVNEEGKAHFVKFHWKPVLGVHSLVWDEAQTIAGKDPDYHRRDLWEAIERGDEVEYELGVQMIDEEDEFKFDFDILDPTKLWPEEIVPVKIIGKMTLNRNQDNVFAETEQAAFHPGHVVPGIDFTNDPLLQGRLFSYTDTQLIRLGGPNFHEIPINRPVCPFHNNQYDGYHRMTINKGPVAYHKNSLQNNDPAPASEEEGGYVHYEEKVEGKKIRQRSESFNDHYSQAKLFWNSMSPVEKEHIISAFRFEVGKVKSKDVRRQVVHMFNRVDGELAKQIAAGVGVEPPEKDEGSNVTFKSPALSQENTVKLPQTRTVAILAEQGFDDEDLSRVLKEFKKAGIIPDIVSSALGVIEGAGGTEIEVGNTLQTVDSVLYDAVYIPGGQESIERLKLHKAASDFVNEAFGHYKAVGAAGEGVDLLLSAAGAHAASQPGIITSRGDKNKDDFGKKLVEAISGHRHWDRQV

SEQ ID NO: 113 - DNA, 1255 bp, Bacillus licheniformis
  gene (1)..(1255): opuAB
accgtagaagagaatgttgtcctgacggatatctttgaattggtctctgatgccagcattccgattgctgtcgtcgatgaaaaacagcggatgaaagggatcatcgtcaaaggcgcattaatcggagcgcttgccggaaatgaccggtatattaatgctcatgaaccagcccatgaagctgttattcaggaggtgagataatgtttactttatcatcgcttccccgcattcctttagccgattggatcgatcagtttgttgactggctgacggtgtcgttcggcggtgtatttaacggaattgccagcggagtggaatcgtttgttgatgagattgtccaagggctgggttttgttccgccggttatgttgaccgttatttttgccgcgctcgtctggctgatcagcaaaagaggcattgcgctgtttactttaatcggattttttatcattgattatttaggatattgggacgccatgctccagacattggcgcttgtcgtcacatccgttctgatttctgtcattatcggcattccggtcggaatctggggctcccaaaagaaaacggttcggagagtcgttacgcccatccttgacttaatgcagacaatgccggcattcgtttatttacttccggccatcttctttttcaatatcggggttgtgccgggtgttgtcgcttccgttatctttgcgatgcctccgacaatccggatgacgatcttagggatccagcaggtgcctgaagatctgattgaagctacagaagcatttggttcaacgacaagccagcggctctttaaagtgcagctgccgctcgcgacgaaaaccattctggccggtatcaatcaaagcatcatgcttgctttgtcaatggtcgttattgcatcaatggtaggcgcgcccggactcggagaagaagtttaccgggctgtgacccagctgcaaacgggtaccggtgtagaaaccggacttgcgattgttattattgcgatcgtacttgaccgggttacacaaaatataaaattgaaaaagagcagggggaatgcataatgtggaaaaagatagcgggaatcggcactgcagcggtactcacattgggtctggctgcctgcggaagcagcaataataatgaaaatgcgtcagtaggcgatcaagtcaattataaaattaccggcattgatcctggagcgggcattatgaatgcgacggaccaggcgctcaaggactacgatctcagcaagtggactgta

SEQ ID NO: 114 - protein, 284 aa, Bacillus licheniformis
MFTLSSLPRIPLADWIDQFVDWLTVSFGGVFNGIASGVESFVDEIVQGLGFVPPVMLTVIFAALVWLISKRGIALFTLIGFFIIDYLGYWDAMLQTLALVVTSVLISVIIGIPVGIWGSQKKTVRRVVTPILDLMQTMPAFVYLLPAIFFFNIGVVPGVVASVIFAMPPTIRMTILGIQQVPEDLIEATEAFGSTTSQRLFKVQLPLATKTILAGINQSIMLALSMVVIASMVGAPGLGEEVYRAVTQLQTGTGVETGLAIVIIAIVLDRVTQNIKLKKSRGNA

SEQ ID NO: 115 - DNA, 1318 bp, Bacillus licheniformis
  gene (1)..(1318): pstS
cagaataggactttttcattaaaacacctgcattgaaccatgcaggtgtttttttgtgcgcccgttcttttcgtgcgcattctatacaaagttcaagcttaatttacaaaaccttaataattcatttaaacaagattaatactgaaaagttacgctatacccgtaggacaaattacacatttacgcgggaggattaagaagtgaaaccatttaaaaaaatcacactgatgttcatcatgtccgttttagttgtcttcgcagcagcttgtggaagcagcagtacaaacaacaatgcagacgaaggcaaacaaagcgataaaacagagaataaggcttcaggttctttgacgatttcgggttcttcggcgatgcagcctcttgtcttagcagcggctgaaaaattcatggatgaaaatcctgatgccgatattcaagttcaggctggaggttctggtacaggcctttcccaagtggctgaaggcacagtccaaatcggaaactccgatgtctttgccgaagaaaaagaaggcattcctgccaaagaacttaccgatcatcaagttgcggttgtcggaatggcagctgccgttaaccctgaagtaggcatcgatgacatttcgttagaagatctcaaaaaggtattcacaggtaaaatcaaaaactggaaagaactaggcgggaaagatcaaaagatcacccttgtcaaccgtcctgattcttcaggaacccgcgcgacttttgtcaaatatgcacttgatggagagactccagctgaaggaatcacggaagattcttcaaacacagtgaaaaagttaatcgctgaaacgcctggtgcaatcggatatcttgcgttctcgtatatcaaggatgatactgtcactccgctttcaatcgacggcgttaagccgacagccgaaaatgtacaaaaaggcaaattcccgatctgggcttaccagcattcttatacgaaaggcgaacctgaaggcttggcgaaagaattcctcgactatttgatgagcgatgacattcaaaaatccatcgtgacggagcagggctacattccagtaacagacatggaagtaaaacgcgacgcggatggaaagcaaactgataaataatgacaatagaaggcgggaaagtgtgctcctgacactttccctcttatgacttgttcttataggagtgtaaaaaatggttcaaaaaatagagaaactgtccgcgaaagagcggctgatcagctccaagcataatcgcaaaatggatgaaatccgcgggaggattttggttgcgtcgtgcgcctttatcatgatggcggcag

SEQ ID NO: 116 - protein, 305 aa, Bacillus licheniformis
VKPFKKITLMFIMSVLVVFAAACGSSSTNNNADEGKQSDKTENKASGSLTISGSSAMQPLVLAAAEKFMDENPDADIQVQAGGSGTGLSQVAEGTVQIGNSDVFAEEKEGIPAKELTDHQVAVVGMAAAVNPEVGIDDISLEDLKKVFTGKIKNWKELGGKDQKITLVNRPDSSGTRATFVKYALDGETPAEGITEDSSNTVKKLIAETPGAIGYLAFSYIKDDTVTPLSIDGVKPTAENVQKGKFPIWAYQHSYTKGEPEGLAKEFLDYLMSDDIQKSIVTEQGYIPVTDMEVKRDADGKQTDK

SEQ ID NO: 117 - DNA, 1117 bp, Bacillus licheniformis
  gene (1)..(1117): purC
cagcttgtcgaggctgaggaaaaaattacgtcccgactgtctccggaacaaattaacgactgctttgattacaattaccacttgaaaaatgtcgactacatttttgaacgtttaggcttggcatagaatagaccgcccgcttttacaggcgggggtctaagtgcttaaatatttccaacattcgggttaggaggccttctgtgaatattgtgaaaaatgaacttttatacgaaggaaaagcgaaaaagatttaccggacggatgatgacaacactttgtttgtcgaatacaaaaactcagcgacggcttttaacggagaaaaaaaagccgagattgacggaaaaggccgtttaaataacgaaatttccagcctgattttcacaaggcttcacgagaagggaatcaacaaccactttgttgaacgaatttctgaaacggagcagctcattaaaaaagtgacgatcatcccgcttgaagtcgttgtccgcaatgtcgttgccggaagcatgtccaaaaggctcggcattccggaaggcacggagctggggcagccgattatcgagttttactacaaggatgacgcgctcggtgatccgttgatcacagaagaccatatccggattttaaaagcagctgagcctgaacaggtggagaaaatcaaatcgattacgagacaggtaaacgaagagctgcagacgattttcgcggcttgcgatgtcagattgatagatttcaagcttgagttcgggctcgataaagacggcgaggtcctgctggccgatgagatttccccggatacgtgccggctgtgggatcaagaaacgaatgaaaagcttgataaagatgtgttcagaagaaatctgggcggattgacggatgcatacgaagaaatattcaaaagacttggaggcatgtaatatgtacaaagtgaaagtgtatgtcagcttaaaagagagtgttttagatcctcaggggagcgccgttcagcatgctttgcacagcatgtcctacaaagaagttcaggacgtccgcatcggcaaatacatggagctcacaatcgaaaaatcggaccgcgaccttgacgttctcgtcaaagaaatgtgcgaaaaacttttgg

SEQ ID NO: 118 - protein, 238 aa, Bacillus licheniformis
VNIVKNELLYEGKAKKIYRTDDDNTLFVEYKNSATAFNGEKKAEIDGKGRLNNEISSLIFTRLHEKGINNHFVERISETEQLIKKVTIIPLEVVVRNVVAGSMSKRLGIPEGTELGQPIIEFYYKDDALGDPLITEDHIRILKAAEPEQVEKIKSITRQVNEELQTIFAACDVRLIDFKLEFGLDKDGEVLLADEISPDTCRLWDQETNEKLDKDVFRRNLGGLTDAYEEIFKRLGGM

SEQ ID NO: 119 - DNA, 988 bp, Bacillus licheniformis
  gene (1)..(988): purN
ccgttcattcaggagaaaggcggcctgaaatccgaggaaatgtttaatgttttcaatatgggaatcggctttgttcttgcggtgaaagaagaggatatgacagacgtgatccaaacgctcgaaaacaacggtgaaaaagcctatctcatcggcagagtgaaagccggaagcggcgttgtgtttggcggtgccggtctttcatgaaaaaattcgcggtatttgcatcaggaagcggaacgaattttgaagcaatcgaaagaagaatgagagaagagaactgggatgccgaaattgtcctcgtcgtctgtgacaagcccgatgcgaaagtgcttgaaagggcggaaaaagccggaatcccgtcgttcgccttccagccgaaatcgttcgacaataaagctgcatttgaacaagtgatcgttgaacagctccgccttcacggagcggaatggattgtgctggcggggtatatgaggctgatcggcgatacgcttctttcggcttaccggaataaaatcatcaacatccatccgtcgctcctgccggcgtttcccggcatcgacgcggtcggacaagcgtacagagcaggcgtgaaagtggcaggcatcacggttcactacgttgacgaaggcatggacaccggtccgattatcgcccagcgcgccgttgagcttgaaaaaagcgaaacactcgcgtctcttgaagaaaagatacacaaattggagcatgaactttatcccgaagtaatcaaagagcttcttgaactaaatggcagaggtgaaaaggaatgacgatcaaacgcgcattaatcagcgtatctgataaaacgaacattgtcccttttgtaaaagaattgacagagcttggcgttgaagtgatttcaacagggggcacaagcaaacttcttcaggaaaacggcgttgacgtcatcggcatttctgaagtgacgggctttcccgaaatcatggacggccgtctgaaaacccttcat

SEQ ID NO: 120 - protein, 195 aa, Bacillus licheniformis
MKKFAVFASGSGTNFEAIERRMREENWDAEIVLVVCDKPDAKVLERAEKAGIPSFAFQPKSFDNKAAFEQVIVEQLRLHGAEWIVLAGYMRLIGDTLLSAYRNKIINIHPSLLPAFPGIDAVGQAYRAGVKVAGITVHYVDEGMDTGPIIAQRAVELEKSETLASLEEKIHKLEHELYPEVIKELLELNGRGEKE

SEQ ID NO: 121 - DNA, 1705 bp, Bacillus licheniformis
  gene (1)..(1705): pyrP
gataatcctataatccttttttaaatgcattccagtgagtttgcaaagagggatggaacgaaagaaagccgccttaccgtgcggcggtctttgtctgcctctttgcgcaatttaagcaaagaggcttttttaatgggcataattcatttcaggatatgaagacatcatagacagtgagtcgatcatcaggaggaaaaaatatgagtcagaaaaaagtgaaccttggtgtcagagaagtaccaaaacctttgaattggatatctttcagcctgcagcatctgtttgccatgttcggagcgacgatccttgtgccgaagcttgtaggcttaagcccgggcgtggccctcattacaagcgggcttggaacgcttgcgtatctcttgattacaaaaggacagatcccggcttacttaggttcatcctttgcatttattgccccgattatcgcggcaaaagccgccggaggccctggcgcggccatgatcggcgcatttatggcaggcatcgcctacgggctgatcgcccttctcatccagcagcttggaacggactggctgatgaggcttctgccgcctgttgtcgtcggtccggtcattatcgtcatcggactcgggctggcaagcacggctgtcaatatggccatgtatgaagattcgggagcaaaagtgctcgtctacagcgccaagcattttggcgtcgcaggattcacactcgcgctcaccataatctgcgcgattttcttgagaggatttttcagcctgattcctgtcttgatcggaattatcggcggatacttgttcgccctgacccagggaatcgtcgacttccagatggttcttgacgccaaatggtttgctgttccggacttcgtgcttccgtttgtcgactatacgccgagcgcggcggctgtcggaatcggaatcgccatggtgcccgtcgctttcgtgacaatgtcagaacatatcggacaccaaatggtattaagcaaggtcgtcggccaggattttattaagaagcccgggcttcaccgctcgattttcggcgacagcgtcgcaacgatgcttgcatcatgcatcggcggacccccgacaacgacttacggagaaaacatcggcgtactcgccatcacaagagtattcagcgtatttgtcatcggcggagcggctgtcattgcactgtgcttcggatttatcggcaaaatttcggccctgatcagctctgtcccttccgcagtcatgggaggcgtttccttcctgctgttcggtatcatcgcatccagcggactgcgcatgctgatcgataataaaattgacttggaaaacaagcgaaacctgatcatctcatccgtcattctcgtcatcggagtgggcggcgcatttatcaagatctcagggtttgagctgtcaagcatggcgcttgcggcgatcgtcggagtggttttaaaccttgtcctgcctgaatcaaagggagatcaggacggaattgaaatagaagagcatatttaaaaaccttttaaatgaagtccagagaggcttgaaagggttgcggagagagaagtgcaaaaaagcgcattctctgattcactgtaccccaagccgttatggcctggggtttttttacgggaaattaagcctgagggggagaaaaaatgaaacacttgaccgcaatgaatgagcttagtcttgaggatattcacgagctgatt

SEQ ID NO: 122 - protein, 434 aa, Bacillus licheniformis
MSQKKVNLGVREVPKPLNWISFSLQHLFAMFGATILVPKLVGLSPGVALITSGLGTLAYLLITKGQIPAYLGSSFAFIAPIIAAKAAGGPGAAMIGAFMAGIAYGLIALLIQQLGTDWLMRLLPPVVVGPVIIVIGLGLASTAVNMAMYEDSGAKVLVYSAKHFGVAGFTLALTIICAIFLRGFFSLIPVLIGIIGGYLFALTQGIVDFQMVLDAKWFAVPDFVLPFVDYTPSAAAVGIGIAMVPVAFVTMSEHIGHQMVLSKVVGQDFIKKPGLHRSIFGDSVATMLASCIGGPPTTTYGENIGVLAITRVFSVFVIGGAAVIALCFGFIGKISALISSVPSAVMGGVSFLLFGIIASSGLRMLIDNKIDLENKRNLIISSVILVIGVGGAFIKISGFELSSMALAAIVGVVLNLVLPESKGDQDGIEIEEHI

SEQ ID NO: 123 - DNA, 1198 bp, Bacillus licheniformis
  gene (1)..(1198): sigB
cgtcgccgatcagggagacagttttgatattcaggataaacagaaggagctcggaccatattcccctgagcatacggtagatcaattgtcagaaggagggctcggtttatatttaatggagacgctcatggatgaagtgcatgtccagattgactcaggcgtaaccgtctcgatgacaaagttcttaaacagggagcgagttgatgatggcacaaccgtccaaaactacgaaactaactaaaaccgaagtcgacacgctgattaaaaaatatcaggagaaccaggatgaagacgctcagctgacccttgttcaaacatataaaaatctggtggaaacactcgccaaaaaatattcaaaaggaaaaagctttcacgaagatctatgccaggttgggatgctcggattgcttggagccatcaaacgctatgaccccgatgtgggaaaatcgtttgaagcgtttgccattccgaccatcatcggagaaatcaaacggtttctccgagataagacctggagcgtccacgttccgagaaggatcaaggagctcgggccgagaatcaaaatggcggtggatcatttaacgactgaaacgcaaaggtctccaagagtcgatgagattgccgagtttcttgacgttacagaagaagaagtgctggaaacgatggagatgggcaaaagctatcaggcgctttccgtcgaccacagcattgaagctgatgcagacggcagcacggtgacgatcttggacatcgtcggctcgcaggaggaaggctatgaaaaggtgaaccaaaagctgatgcttgaaagcgtattacacgtactatccgaccgtgaaaagcaaatcattgagctgacttacattcaaaacaaaagccaaaaagaaaccggcgaaatgctgggcatatcgcaaatgcacgtctcacggctgcagcgcaaagctgtcaaaaggctgagggaagcattggctgaagatccgtatatggagcttaaaccatgatttggactgagtcgaatgaacacatgcaagcgcttgtttatcagcttcctaaagaaggaaaagccgtctgcggtgacagtttttatttccatgcgactgacgaaaccatggtgtgcgcactggctgacgggcttggcagcggtgcgttggcacgcgaatcctcttcggtcatcagcgaagttgtcgaagagcacggccat

SEQ ID NO: 124 - protein, 265 aa, Bacillus licheniformis
LMMAQPSKTTKLTKTEVDTLIKKYQENQDEDAQLTLVQTYKNLVETLAKKYSKGKSFHEDLCQVGMLGLLGAIKRYDPDVGKSFEAFAIPTIIGEIKRFLRDKTWSVHVPRRIKELGPRIKMAVDHLTTETQRSPRVDEIAEFLDVTEEEVLETMEMGKSYQALSVDHSIEADADGSTVTILDIVGSQEEGYEKVNQKLMLESVLHVLSDREKQIIELTYIQNKSQKETGEMLGISQMHVSRLQRKAVKRLREALAEDPYMELKP

SEQ ID NO: 125 - DNA, 715 bp, Bacillus licheniformis
  gene (1)..(715): trxA
ctgaaatggccggcttttgacataggcacatttccgccttctgcaaaataacatgggatatggacattttcagaaaggctaaaatcccgatgtgtaaaactgtgcagatagggaaacgtataatcaagcattatgttagcgtgaaccaaagggagatgctatactaaaaagcataatttcacaattggaggaatgaaagaatggctatcgtaaaagcaactgatcaaacattctcatctgaaacaagcgaaggcgtagttcttgccgacttttgggctccttggtgcggaccttgtaaaatgatcgcgccggtacttgaagagcttgatcaagaaatgggagacaaactgaaaatcgtaaaaatcgatgttgacgaaaaccaagaaactgccggcaaatacggcgtgatgagcatcccgactctcctcgtactaaaagacggcgaagtggtcgaaacatctgtcggctttaaaccgaaagaagcgcttgaagaactggtcaacaaacatttgtaaaatagaaacctgtgaaaaaggcgattccaaagggatcgcttttttcttacaagtcatcttctttctccatttgtggcgaacgtacggtcggtcatggtaaacttaaatgaaggaaaaacgccgaaataaggaaggatttttgatgaacaaaaaaatcaaagaaaagctggcccttcttccggatcagcctggctgctact

SEQ ID NO: 126 - protein, 104 aa, Bacillus licheniformis
MAIVKATDQTFSSETSEGVVLADFWAPWCGPCKMIAPVLEELDQEMGDKLKIVKIDVDENQETAGKYGVMSIPTLLVLKDGEVVETSVGFKPKEALEELVNKHL

SEQ ID NO: 127 - DNA, 21 bp, Artificial
  Primer
gctttaccgttctgctattgg

SEQ ID NO: 128 - DNA, 44 bp, Artificial
  Primer
ctaatacgactcactatagggagaagttgatttcgatacggcgc

SEQ ID NO: 129 - DNA, 19 bp, Artificial
  Primer
gggtaaaataatggtatcg

SEQ ID NO: 130 - DNA, 44 bp, Artificial
  Primer
ctaatacgactcactatagggagactttgatgttcatgtgtttc

(19) United States
(12) Patent Application Publication
(10) Pub. No.: US 2006/0040279 A1
(43) Pub. Date: Feb. 23, 2006

(54) DNA chips used for bioprocess control

(75) Inventors: Joerg Feesche, Erkrath (DE); Karl-Heinz Maurer, Erkrath (DE); Roland Breves, Mettmann (DE); Thomas Schweder, Greifswald (DE); Michael Hecker, Greifswald (DE); Britta Juergen, Greifswald (DE); Birgit Voigt, Greifswald (DE)

Correspondence Address: WOODCOCK WASHBURN LLP, ONE LIBERTY PLACE, 46TH FLOOR, PHILADELPHIA, PA, 19103, US

(73) Assignee: Henkel Kommanditgesellschaft auf Aktien, Duesseldorf (DE)

(21) Appl. No.: 11/077,619

(22) Filed: Mar. 11, 2005

Related U.S. Application Data
(63) Continuation of application No. PCT/EP03/09979, filed on Sep. 9, 2003.

(30) Foreign Application Priority Data
Sep. 11, 2002 (DE) 10242433.0

(51) Int. Cl. C12Q 1/68; C07H 21/04; C12P 21/06; C12N 9/88; C12N 15/74

(52) U.S. Cl. 435/6; 435/69.1; 435/232; 435/252.3; 435/471; 536/23.2; 435/287.2

(57) ABSTRACT

The present invention provides methods for determining the physiological state of cells isolated from an organism of interest utilizing chips to which nucleic acid probes are attached. In preferred embodiments of the invention, the cells undergo a biological process and the physiological state of the cells is determined at various points in time throughout the biological process.

### CROSS REFERENCE TO RELATED APPLICATIONS

This application is a continuation of PCT/EP2003/009979, filed Sep. 9, 2003, which claims priority to DE 102 42 433.0, filed Sep. 11, 2002, the disclosures of which are incorporated herein in their entireties.

The present invention relates to chips doped with nucleic-acid probes, which are suitable for monitoring the course of bioprocesses, and to the use of corresponding probes on such chips, and to processes and possible uses based on chips of this kind, and to genes suitable therefor.

The industrial utilization of biological processes is faced with the very fundamental problem of monitoring the course of said processes in order to attain the desired result, to conserve resources and/or to achieve an optimal result within a given time. Biological processes mean, for example, culturing microorganisms on an agar plate or in a shaker culture, but in particular fermenting said microorganisms and, respectively, obtaining raw materials via fermentation of microorganisms. To this end, there is extensive prior art, with regard to both unicellular eukaryotes such as yeasts or streptomycetes and Gram-negative or Gram-positive bacteria.

Processes of this kind are monitored firstly by observing the properties and requirements of the relevant organisms, which change in the course of the process, these changes being reflected, for example, in the optical density and viscosity of the medium, in absorbed or released gases, in pH changes or in changing nutrient requirements. The measurement of enzymic activities via suitable assays, for example the detection of activities of interest in the culture supernatant, may also be included here.

Secondly, various techniques have been developed in recent years, in order to follow the metabolic processes of the organisms in question at the level of gene expression. A common method for this is the use of genes for readily detectable proteins as indicators of the activity of the promoters of the actual genes of interest (promoter analysis, gene expression analysis).

For this, appropriate apparatus (“(bio)sensors”) have been developed in order to obtain a result as close to real time as possible. An overview over the application of sensor technology to biological questions is given, for example, in the article “Biosensor Microsystems” by G. Urban (2001) in Sensors Update, 8, pp. 189-214.

The study “On-line monitoring of gene expression” (I. Biran et al., 1999, Microbiology, 145, pp. 2129-2133), for example, describes an electrochemical sensor for online analysis of E. coli cultures. According to this, the lacZ gene can be put under the control of the promoter of the RpoS-dependent osmY gene which is expressed when a culture enters the stationary growth phase. The β-galactosidase activity derived therefrom, which appears in the culture medium, may be determined via an electrochemical sensor. The signal obtained therewith thus indicates the end of the exponential growth phase of the culture in question.

Other techniques are concerned with detecting the mRNA of interest, or the derived proteins themselves. These techniques include (1.) proteome analysis, i.e. observing the change in provision of the cells in question with proteins, which analysis is usually carried out by way of two-dimensional gel electrophoresis of cell lysates, (2.) analysis of the mRNA formed (transcriptome) by way of a “genomic DNA array” produced in an analogous manner, and (3.) chip technology.

The latter is in a comparatively early stage of development. While the two methods mentioned first are ultimately based on quantitative isolation procedures and time-consuming analyses of the macromolecules in question, the chip technology is based on the principle of attaching on physically readable carriers (chips) probes for proteins or for nucleic acids, which respond immediately to the presence of the proteins or nucleic acids in question. Compared to the two technologies mentioned earlier, chips of this kind are hoped to provide an analysis close in time to the relevant process (at line analysis). Another advantage is the need for comparatively small amounts of sample.

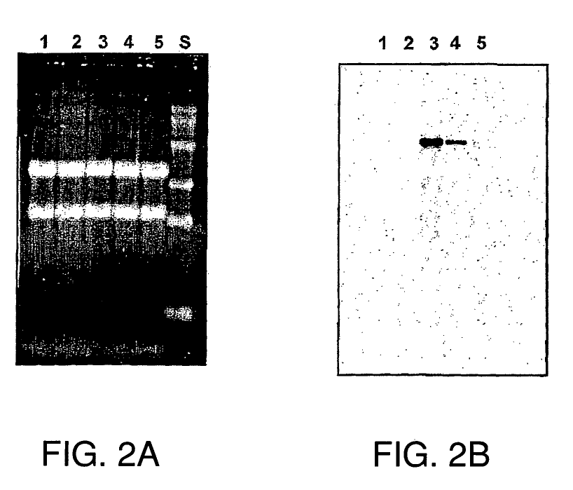

The principle of chip-based measurements is introduced, for example, diagrammatically in FIG. 2 of the article “Real-time electrochemical monitoring: toward green analytical chemistry” by J. Wang (Acc. Chem. Res.; ISSN 0001-4842; Rec. Sep. 12, 2001, S. A-F). According to this, the sample to be analyzed is contacted with a biorecognition layer which may be, for example, an enzyme, an antibody, a receptor or DNA; the signal received therewith is emitted as voltage or electric potential via a transducer, for example an amperometric or potentiometric electrode, through an amplifier (amplification/processing). The study in question also mentions optical systems compared to which the electronically analyzable systems were regarded by the author as being superior with respect to miniaturizability and other advantages.

Thus, the prior art has a broad range regarding the structure and function of such chips: a fundamental distinction is made between protein-binding chips and chips recognizing nucleic acids, i.e. in particular mRNA. Owing to the present invention, the protein-specific chips need not be considered. mRNA-recognizing chips are usually doped with complementary DNA molecules. The DNA chip analyses include those with PCR amplification of the target sequence and those without amplification. There are also those with optical evaluation of the signals attributable to the recognition and those with electrical evaluation.

The optical detection methods partly require a mechanism of amplifying the signals. For this purpose, for example, fluorophores, acridinium esters or indirect detection via secondary binding events, for example via biotin, avidin/streptavidin or digoxigenin, are described. In the latter case, optical detection makes use of digoxigenin-specific antibodies which are labeled with an enzyme. Here, the enzyme activity is detected either colorimetrically or by way of luminescence. According to Westin et al. (2000), Nature Biotechnol., 18, pp. 199-204, hybridization may be coupled to a PCR on the DNA chip in order to be able to carry out the entire detection reaction on one chip (“lab-on-a-chip concept”).

Other studies have described the development of DNA chips which miniaturize the principle of capillary electrophoresis for DNA sequencing or separation (Woolley and Mathies (1994), Proc. Natl. Acad. Sci., 91, pp. 11348-11352; Liu et al. (2000), Proc. Natl. Acad. Sci., 97, pp. 5369-5374).

Electrically readable DNA chips have been introduced in principle previously by some publications (Hoheisel (1999), DECHEMA Jahresbericht 1999, pp. 8-11; Hintsche et al. (1997), EXS, 80, pp. 267-283). Wright et al. (2000; Anal. Biochem., 282, pp. 70-79) utilized an ion channel sensor (ICS) for DNA detection, as has been described for the first time by Cornell et al. (1997: Nature, 387, pp. 580-583). This is a process in which the conductivity of molecular ion channels is detected by way of a binding reaction. The sensor is essentially an impedance element. According to Cheng et al. (1998; Nat. Biotechnol, 16, pp. 541-546), it is possible to utilize electrical pulses for amplifying the hybridization reaction on optical DNA chips. Fritsche et al. (2002; Laborwelt II) proposed an electrical chip system which employs metallic nanoparticles bound to oligonucleotides, for example. In this system, “metallic amplification” during the hybridization reaction causes a drop in the electrical resistance at the electrode, which drop can then be measured as a signal.

Another approach is based on an electrical detection principle which uses DNA probes which, due to labeling with a suitable enzyme (e.g. alkaline phosphatase), after hybridization result in an electrically active substrate which can then be detected via a redox reaction at the electrode (Hintsche et al. (1997), EXS, 80, pp. 267-283).

If it is decided to use a particular chip type, for example a chip for monitoring bioprocesses, which responds to nucleic acids, the more specific problem arises as to which gene activities are to be observed. To manufacture and to use the appropriately produced chip, it is then possible to make use of the prior art again.

For technical reasons, the number of genes which can be analyzed simultaneously using one nucleic acid chip is limited. Thus, optically readable chips are currently superior to those which can be evaluated electrically, with regard to the number of probes being able to be applied to the chip. The limits of the latter chips are determined by the miniaturizability of the electronic measuring units.

Thus the biological problem arises, as to which gene activities depict the relevant process. This also includes monitoring product formation, if, for example, said product is produced fermentatively. At the same time, however, control genes should also be included which indicate if the process develops in a direction which is not intended. In the course of this monitoring, on the one hand, for reasons of practicability, the number of different genes observed should not be too high. On the other hand, recording a broad spectrum of gene activities by using one and the same chip is desirable, for example in order to recognize a multiplicity of possible scenarios, but also, for example, if a plurality of organism strains are to be observed in parallel or the same host is to be utilized for the formation of different products, so that it is not necessary to develop a new chip each time.

Of particular industrial interest are biotechnological processes using Gram-positive bacteria, since the latter are used for industrial production of desired substances, particularly owing to their secretion capability. Among said bacteria, those of the genus Bacillus and among these in turn the species B. subtilis, B. amyloliquefaciens, B. agaradherens, B. licheniformis, B. lentus and B. globigii are currently economically the most important.

The studies introduced below and subsequently summarized in table 1, for example, are concerned with simultaneous observation of the activity of a plurality of genes in bacteria (multiparametric recording).

The article “Monitoring of genes that respond to process-related stress in large-scale bioprocesses” by Schweder et al. (1999), Biotech. Bioeng., 65, pp. 151-159, describes the alteration in mRNA levels of various stress factor-inducible genes, namely clpB, dnaK (induced during heat shock), uspA (glucose deficiency), proU (osmotic stress), pfl and frd (O₂ deficiency) and ackA (glucose surplus) in the course of a fermentation of E. coli and during the subsequent concentration phase. Said genes were recorded via a PCR-based method carried out in a conventional matter. In this connection, different rates of expression were detected already at various sites in the reactor, as were responses to altered conditions, which took place in a matter of seconds. The genes proU and ackA were very active during growth, but distinctly less so with glucose deficiency. In contrast, the genes clpB, dnaK, pfl and frd remained constant during growth and exhibited increased expression with glucose surplus and (related therewith) O₂ deficiency. uspA remained comparatively constant both with growth and with glucose deficiency. The starting point of this study was the idea of using said genes as indicators for monitoring a bioprocess; however, at least for uspA, these hopes were dashed.

Another fermentation of E. coli is described in the study “Monitoring of genes that respond to overproduction of an insoluble recombinant protein in Escherichia coli glucose-limited fed-batch fermentations” by Jürgen et al. (2000), Biotech. Bioeng., 70, pp. 217-224. Here, expression of the genes Ion, dnaK, ibpB, htrA, ppiB, groEL, tig, s6, 19 and dps is observed partly at the mRNA level, partly at the protein level, partly at both levels. The investigation was carried out by way of 2D PAGE and DNA array technique. In view of the results which are compiled in table 1 of the present application, it is suggested to monitor recombinant bioprocesses such as heterologous protein preparation via (directly) process-relevant proteins and reporter genes such as ibpB.

The study “Genomic analysis of high-cell-density recombinant Escherichia coli fermentation and “cell conditioning” for improved recombinant protein yield” by R. T. Gill et al. (2001; Biotech. Bioeng., 72, pp. 85-95) is concerned with another observation of the course of a fermentation in which a recombinant protein is expressed by E. coli. This study describes increased expression of the stress genes degP, uvrB, alpA, mltB, recA, ftsH, ibpA, aceA and groEL under the conditions mentioned with high cell density, compared to low cell density. Said genes were grouped among each other into certain clusters, according to the strength of the reaction. This was determined via an approach based on RT-PCR and DNA microarray, which was supplemented by dot blot analysis and which was applied to samples from two points in time of the fermentation, that is at the beginning, at low cell density, and towards the end, at high cell density. From this, cell conditioning approaches were developed in order to reduce the stress response of the cells.

Fundamental differences in the expression patterns of Gram-positive organisms compared to those of Gram-negative bacteria are reviewed by the study “Proteome and transcriptome based analysis of Bacillus subtilis cells overproducing an insoluble heterologous protein” by Jürgen et al. (2001), Appl. Microbiol. Biotechnol, 55, pp. 326-332. Said study describes expression inter alia of the genes dnaK, groEL, grpE, clpP, clpC, clpX, rpsB and rplJ in B. subtilis, as can be determined via the DNA macroarray technique and via two-dimensional polyacrylamide gel electrophoresis. According to this, the genes for purine synthesis and pyrimidine synthesis and those of particular ribosomal proteins are expressed in Gram-positive bacteria used for overexpression more strongly than was to be expected, owing to the findings in the case of Gram-negative bacteria. Another difference relates to the proteases Lon and Clp.

Table 1: Genes whose change in expression during fermentations has been described in the documents mentioned

Abbreviations: Glc: glucose; σ: respective transcription factor of Gram-negative bacteria; +: increased mRNA level; −: reduced mRNA level;

It is noted in each case when the assay was only for a change in the protein level.

| | | Description | | | |
|---|---|---|---|---|---|
| | | | | Gill et al., 2001: | |
| | | | | Expression | |
| | | | | during | |
| | | | Jürgen et | protein | Jürgen et al., |
| | | Schweder et | al., 2000: | production in | 2001: |
| | | al., 1999: | Expression | E. coli at | Expression |
| | | Expression | during | high, | during |
| | | during | protein | compared to | protein |
| | Signal | fermentation | production | low, cell | production in |
| Gene | function | of E. coli | in E. coli | density | Bacillus |
| aceA | Stationary | | | + | |
| | phase | | | | |
| ackA | Induced | high with | | | |
| | with Glc | growth; | | | |
| | surplus | drastically | | | |
| | | lower with Glc | | | |
| | | deficiency | | | |
| acnB | Citrate cycle | | + (only | | |
| | | | initially; at | | |
| | | | protein level) | | |
| alpA | DNA lesion | | | + | |
| clpB | Heat shock- | relatively | + | | |
| | induced; | constant with | | | |
| | σ³²- | growth and | | | |
| | dependent | with Glc | | | |
| | | deficiency; | | | |
| | | increased with | | | |
| | | Glc surplus | | | |
| | | and O₂ | | | |
| | | deficiency | | | |
| clpC | Heat shock | | | | + |
| | III | | | | |
| clpE | Heat shock | | | | + |
| | III | | | | |
| clpP | Heat shock | | | | + |
| | III | | | | |
| clpX | Heat shock | | | | + |
| | IV | | | | |
| degP | Chaperone, | | | + | |
| | protease; | | | | |
| | heat shock | | | | |
| dnaK | Heat shock I | relatively | + (only | | + |
| | (or σ³²- | constant with | initially) | | |
| | dependent) | growth and | | | |
| | | with Glc | | | |
| | | deficiency; | | | |
| | | increased with | | | |
| | | Glc surplus | | | |
| | | and O₂ | | | |
| | | deficiency | | | |
| dps | Sigma- | | − (at protein | | |
| | dependent | | level) | | |
| | protein (σ³⁸- | | | | |
| | dependent) | | | | |
| frd | Induced | relatively | | | |
| | with O₂ | constant with | | | |
| | deficiency | growth and | | | |
| | | with Glc | | | |
| | | deficiency; | | | |
| | | increased with | | | |
| | | Glc surplus | | | |
| | | and O₂ | | | |
| | | deficiency | | | |
| ftsH | Protease, | | | + | +/− |
| | DNA lesion; | | | | |
| | heat shock | | | | |
| | IV | | | | |
| glcB | Glyoxalate | | + (at protein | | |
| | cycle | | level) | | |
| gltA | Citrate cycle | | + (only | | |
| | | | initially; at | | |
| | | | protein level) | | |
| groEL | Chaperone; | | + | + | + |
| | heat shock I | | | | |
| | (σ³²- | | | | |
| | dependent) | | | | |
| groES | Heat shock I | | + (at protein | | + |
| | (or σ³²- | | level) | | |
| | dependent) | | | | |
| grpE | Heat shock I | | | | + |
| gsiB | Heat shock II | | | | +/− |
| gspA | Heat shock II | | | | +/− |
| htpG | Heat shock | | | | +/− |
| | IV | | | | |
| htrA | Periplasmic | | − | | |
| | protease | | | | |
| | (σ²⁴- | | | | |
| | dependent) | | | | |
| ibpA | Inclusion | | + (at protein | + | |
| | body- | | level) | | |
| | associated | | | | |
| | protein A; | | | | |
| | chaperone | | | | |
| | (σ³²- | | | | |
| | dependent) | | | | |
| ibpB | Inclusion | | + | | |
| | body- | | | | |
| | associated | | | | |
| | protein B | | | | |
| | (σ³²- | | | | |
| | dependent) | | | | |
| idh | Citrate cycle | | + (at protein | | |
| | | | level) | | |
| Ion | Heat shock | | + (only | | |
| | (σ³²- | | initially) | | |
| | dependent) | | | | |
| IonA | Heat shock | | | | +/− |
| | IV | | | | |
| IonB | Heat shock | | | | − |
| | IV | | | | |
| mdh | Citrate cycle | | + (only | | |
| | | | initially; at | | |
| | | | protein level) | | |
| mltB | Cell lysis | | | + | |
| ompT | Outer | | +/− (at | | |
| | membrane | | protein level) | | |
| | protease | | | | |
| | (σ⁷⁰- | | | | |
| | dependent) | | | | |
| osmY | σ³⁸- | | − | | |
| | dependent | | | | |
| pfl | Induced with | relatively | | | |
| | O₂ deficiency | constant with | | | |
| | | growth and | | | |
| | | with Glc | | | |
| | | deficiency; | | | |
| | | increased with | | | |
| | | Glc surplus | | | |
| | | and O₂ | | | |
| | | deficiency | | | |
| ppiB | Peptidyl- | | − | | |
| | prolyl cis- | | | | |
| | trans | | | | |
| | isomerase | | | | |
| | (σ⁷⁰- | | | | |
| | dependent) | | | | |
| proU | Induced with | high with | | | |
| | osmotic | growth; | | | |
| | stress | drastically | | | |
| | | lower with | | | |
| | | Glc deficiency | | | |
| purB | Purine | | | | + |
| | synthesis | | | | |
| purC | Purine | | | | + |
| | synthesis | | | | |
| purM | Purine | | | | + |
| | synthesis | | | | |
| pyrA | Pyrimidine | | | | + |
| | synthesis | | | | |
| pyrD | Pyrimidine | | | | + |
| | synthesis | | | | |
| recA | DNA lesion | | | + | |
| rplI | rib. protein | | − (at protein | | |
| | (protein | | level) | | |
| | synthesis) | | | | |
| rplJ | rib. protein | | | | + |
| | (protein | | | | |
| | synthesis) | | | | |
| rpoA | σ⁷⁰- | | − | | |
| | dependent | | | | |
| rpoS | σ³⁸- | | − | | |
| | dependent | | | | |
| rpsA | rib. protein | | +/− (at | | + |
| | (protein | | protein level) | | |
| | synthesis) | | | | |
| rpsB | rib. protein | | | | + |
| | (protein | | | | |
| | synthesis) | | | | |
| rpsF | rib. protein | | − (at protein | | |
| | (protein | | level) | | |
| | synthesis) | | | | |
| sucA | Succinyl- | | + (only | | |
| | CoA | | initially; at | | |
| | synthetase | | protein level) | | |
| | (citrate | | | | |
| | cycle | | | | |
| tig | σ⁷⁰- | | + (at protein | | |
| | dependent | | level) | | |
| trxA | Heat shock | | | | +/− |
| | IV; oxidative | | | | |
| | stress | | | | |
| tufB | EF-Tu (σ⁷⁰- | | +/− (at | | |
| | dependent) | | protein level) | | |
| uspA | Induced with | relatively | | | |
| | Glc | constant with | | | |
| | deficiency | growth and | | | |
| | | with Glc | | | |
| | | deficiency | | | |
| uvrB | DNA lesion | | | + | |
| yabE | Secretion | | | | |
| | stress | | | | |
| yclI | unknown | | | | + |
| yfiH | | | | | + |
| yjlC | | | | | + |
| ysxA | | | | | + |
| yumD | | | | | + |
| ywbA | | | | | + |

In principle, all of these genes could be observed in the course of a biological process such as bacterial culture or fermentation and thus would give in each case a very special statement about the particular physiological aspect of the culture in question. Thus, in principle, they could all be utilized in order to monitor such a process. On the other hand, in view of the complexity, the question arises as to which of these indicator genes a corresponding monitoring can be restricted to, without substantial aspects being overlooked. The fewer the genes that need to be observed, the simpler is the manner in which the sensors in question can be prepared and the lower is the technical complexity of the chosen detection system.

Several publications have meanwhile disclosed some of these genes or even chips with some of these genes or indicate at least the possibility of the preparation thereof. Thus, for example, the two patent applications DE 10136987 A1 and DE 10108841 A1 disclose in each case a Corynebacterium glutamicum gene, namely clpC and citB, respectively. Both genes are described as being relevant for the amino acid metabolism, this being the reason for an intended, commercially interesting utilization of said genes, which comprises inactivating or at least attenuating said genes in order to optimize the fermentative production of amino acids by this microorganism. According to these applications, further possible applications may consist of providing probes for the gene products in question on nucleic acid-binding chips.

On the other hand, increasingly more genomic data of various organisms are published, which contain such an abundance of sequence data that a representative selection therefrom seems desirable. Thus the patent application WO 02/055655 A2 discloses more than 1800 DNA sequences which have been determined by completely sequencing the genome of the microorganism Methylococcus capsulatus. These sequences include one of the lipid metabolism, namely eno, which, in addition to, is also of interest within the scope of the present patent application, but which would not have been taken into account only on the basis of this application.

While thus practically all of these genes have been characterized in each case separately or in groups in the prior art and partly have been discussed as indicators for the physiological state of the cells in question, the selection of genes for a control of biological processes, in particular of fermentations of Gram-positive bacteria, carried out for the present invention has not emerged from the prior art.

It was thus the object to identify genes, preferably to identify a representative cross section of genes, which are suitable for indicating, via changes in metabolic activities, how an observed bioprocess proceeds. In particular, attention should be paid here to fermentations, particularly those fermentations which are used for production of biological desired substances. Accordingly, those physiological states which indicate that the cells in question are leaving the path of the optimal growth profile should become visible. Said states include, for example, states of starvation relating to various nutrients or stress situations such as, for example, heat shock or cold shock, shearing stress, oxidative stress or oxygen limitation.

It was the aim to develop probes for said genes in order to be able to use them for monitoring corresponding bioprocesses.

Another object was to develop a sensor doped with probes for a representative selection of marker genes, which is suitable of indicating, when monitoring a bioprocess based on microorganisms, in particular Gram-positive or Gram-negative bacteria, changes in the metabolic activities characterizing said process more rapidly than conventional methods, in particular those based on gel electrophoresis. This should give the advantage of being able to intervene in the process in question with as short a delay as possible, for it should be possible for a bioprocess to be carried out more efficiently due to the possibility of regulations close to real time.

A sensor of this kind should be usable for a plurality of processes comparable with one another and should be adaptable to specific possible uses by means of comparatively slight variations. It should preferably target bioprocesses on the basis of Bacillus species, in particular B. subtilis, B. amyloliquefaciens, B. lentus, B. globigii, and very particularly B. licheniformis. Among bioprocesses, special emphasis was on fermentations, in particular industrial manufacture of products, very particularly of overexpressed proteins.

A sensor of this kind should also make possible corresponding processes for measuring the physiological state of the relevant cells and also corresponding possible uses for monitoring the relevant biological processes.

The first part of this object is achieved by identifying the following genes: acoA, ahpC, ahpF, citB, clpC, clpP, codY, cspA, cspB, des, dnaK, eno, glnR, groEL, groL, gsiB, ibpA, ibpB, katA, katE, IctP, Idh, opuAB, phoA, phoD, pstS, purC, purN, pyrB, pyrP, sigB, tnrA, trxA and ydjF.

The wider object is achieved by a chip which is doped with nucleic-acid probes or nucleic acid-analog probes for at least four of the following genes: acoA, ahpC, ahpF, citB, clpC, clpP, codY, cspA, cspB, des, dnaK, eno, glnR, groEL, groL, gsiB, ibpA, ibpB, katA, katE, IctP, Idh, opuAB, phoA, phoD, pstS, purC, purN, pyrB, pyrP, sigB, tnrA, trxA and ydjF, and for genes regulated identically in the relevant organism from the metabolic pathways characterized in each case by said genes.

Depending on the process to be observed, probes for further genes or gene products may be included.

The genes usable according to the invention for bioprocess control are compiled together with the functions of the proteins derived in each case and the physiological signal which they represent, in table 2 below, if the latter is not unambiguously obvious from the function. In summary, these are genes whose gene products become active in the following metabolic connections: cell wall synthesis, DNA replication, membrane transport mechanisms, carbon metabolism, citrate cycle (tricarboxylic acid cycle; TCA), respiratory chain, nitrogen metabolism, phosphate metabolism, amino acid synthesis, purine synthesis and pyrimidine synthesis, translation, including ribosomal genes, secretion, anaerobiosis and sporulation, if the organisms in question are capable thereof.

In addition, table 2 makes reference to the corresponding DNA and amino acid sequences which may be obtained, for example, from Bacillus subtilis, Escherichia coli and/or B. licheniformis and which are indicated according to this table in the sequence listing of the present application under the numbers SEQ ID NO. 1 to SEQ ID NO. 126.

Table 2: Genes usable according to the invention for bioprocess control, functions of the proteins derived in each case and signals related thereto and references to the identified sequences from the corresponding organisms, as indicated in the sequence listing of the present application.

| | | Provided in the present | |
|---|---|---|---|
| | | application | |
| | | | under (in each |
| | | | case DNA |
| | | | sequence and |
| | | | amino acid |
| Gene | Function and signal | from | sequence) |
| acoA | Acetoin dehydrogenase E1 | B. subtilis | SEQ ID NO. 1, 2 |
| | component (TPP-dependent α subunit; | | |
| | glucose limitation) | | |
| | Acetoin dehydrogenase E1 | B. licheniformis | SEQ ID NO. 83, |
| | component (TPP-dependent α subunit; | | 84 |
| | E.C. 1.2.4.—, glucose limitation) | | |
| ahpC | Alkyl hydroperoxide reductase | B. subtilis | SEQ ID NO. 3, 4 |
| | (small subunit; general stress; | | |
| | stationary phase) | | |
| | Alkyl hydroperoxide reductase (E.C. | B. licheniformis | SEQ ID NO. 85, |
| | 1.6.4.—; small subunit; general stress; | | 86 |
| | stationary phase) | | |
| ahpF | Alkyl hydroperoxide reductase | B. subtilis | SEQ ID NO. 5, 6 |
| | (large subunit)/NADH dehydrogenase | | |
| | (general stress; stationary | | |
| | phase) | | |
| | Alkyl hydroperoxide reductase | B. licheniformis | SEQ ID NO. 87, |
| | (large subunit)/NADH dehydrogegnase | | 88 |
| | (E.C. 1.6.99.3; general stress; | | |
| | stationary phase) | | |
| citB | Aconitate hydratase (citrate cycle- | B. subtilis | SEQ ID NO. 7, 8 |
| | active) | | |
| | Aconitate hydratase (EC 4.2.1.3; | B. licheniformis | SEQ ID NO. 89, |
| | citrate cycle-active) | | 90 |
| clpC | Class III stress response-related | B. subtilis | SEQ ID NO. 9, |
| | ATPase | | 10 |
| | Class III stress response-related | B. licheniformis | SEQ ID NO. 91, |
| | ATPase | | 92 |
| clpP | ATP-dependent protease, proteolytic | B. subtilis | SEQ ID NO. 11, |
| | subunit (class III heat shock protein) | | 12 |
| | ATP-dependent protease, proteolytic | E. coli | SEQ ID NO. 13, |
| | subunit | | 14 |
| | ATP-dependent protease, proteolytic | B. licheniformis | SEQ ID NO. 93, |
| | subunit (class III heat shock protein; | | 94 (incomplete) |
| | E.C. 3.4.21.92) | | |
| codY | Pleiotropic transcriptional repressor | B. subtilis | SEQ ID NO. 15, |
| | (nitrogen metabolism) | | 16 |
| | Pleiotropic transcriptional repressor | B. licheniformis | SEQ ID NO. 95, |
| | (nitrogen metabolism) | | 96 |
| cspA | Cold shock protein CS7.4; similar to | E. coli | SEQ ID NO. 17, |
| | Y-box DNA-binding proteins of | | 18 |
| | eukaryotes; transcription factor | | |
| | (stationary phase) | | |
| cspB | Major cold shock protein (stationary | B. subtilis | SEQ ID NO. 19, |
| | phase) | | 20 |
| | Cold shock protein with similarity to | E. coli | SEQ ID NO. 21, |
| | CspA (stationary phase) | | 22 |
| | Major cold shock protein (stationary | B. licheniformis | SEQ ID NO. 97, |
| | phase) | | 98 |
| des | Membrane phospholipid desaturase | B. subtilis | SEQ ID NO. 23, |
| | (formation of unsaturated fatty | | 24 |
| | acids) | | |
| | Fatty acid desaturase (E.C. 1.14.99.—; | B. licheniformis | SEQ ID NO. 99, |
| | formation of unsaturated fatty acids) | | 100 |
| dnaK | Class I heat shock protein | B. subtilis | SEQ ID NO. 25, |
| | (molecular chaperone) | | 26 |
| | Molecular chaperone of the HSP-70 | E. coli | SEQ ID NO. 27, |
| | type, with DnaJ; stress-related heat | | 28 |
| | shock DNA biosynthesis, ATP- | | |
| | regulated binding and release of | | |
| | polypeptide substrates | | |
| | Class I heat shock protein | B. licheniformis | SEQ ID NO. 101, |
| | (molecular chaperone) | | 102 (7 positions |
| | | | toward the end of |
| | | | the gene |
| | | | uncertain) |
| eno | Enolase (glucose starvation) | B. subtilis | SEQ ID NO. 29, |
| | | | 30 |
| | Enolase (E.C. 4.1.2.11; glucose | B. licheniformis | SEQ ID NO. 103, |
| | starvation) | | 104 |
| glnR | Transcriptional repressor of the | B. subtilis | SEQ ID NO. 31, |
| | glutamine synthetase gene (nitrogen | | 32 |
| | metabolism) | | |
| | Transcriptional repressor of the | B. licheniformis | SEQ ID NO. 105, |
| | glutamine synthetase gene (nitrogen | | 106 |
| | metabolism) | | |
| groEL | Class I heat shock protein | B. subtilis | SEQ ID NO. 33, |
| | (chaperonin) | | 34 |
| | Class I heat shock protein | B. licheniformis | SEQ ID NO. 107, |
| | (chaperonin) | | 108 |
| groL | Chaperone for assembly of the | E. coli | SEQ ID NO. 35, |
| | enzyme complex; phage morphogenesis; | | 36 |
| | large subunit of GroESL | | |
| gsiB | Heat shock II; general stress protein | B. subtilis | SEQ ID NO. 37, |
| | (sigma-B) | | 38 |
| ibpA | Inclusion body-associated protein A; | E. coli | SEQ ID NO. 39, |
| | chaperone, heat-inducible protein of | | 40 |
| | the HSP20 family | | |
| ibpB | Inclusion body-associated protein B; | E. coli | SEQ ID NO. 41, |
| | chaperone, heat-inducible protein of | | 42 |
| | the HSP20 family | | |
| katA | Vegetative catalase 1 (oxidative | B. subtilis | SEQ ID NO. 43, |
| | stress) | | 44 |
| | Catalase (E.C. 1.11.1.6; oxidative | B. licheniformis | SEQ ID NO. 109, |
| | stress) | | 110 |
| katE | Catalase 2 (general stress; SigB- | B. subtilis | SEQ ID NO. 45, |
| | dependent) | | 46 |
| | Catalase hydroperoxidase III | E. coli | SEQ ID NO. 47, |
| | (general stress; SigS-dependent) | | 48 |
| | Catalase (E.C. 1.11.1.6; oxidative | B. licheniformis | SEQ ID NO. 111, |
| | stress) | | 112 |
| IctP | L-lactate permease (induced by | B. subtilis | SEQ ID NO. 49, |
| | anaerobiosis; repressed by nitrate) | | 50 |
| Idh | L-lactate dehydrogenase (induced by | B. subtilis | SEQ ID NO. 51, |
| (=IctE) | anaerobiosis; repressed by nitrate) | | 52 |
| opuAB | Glycine-betaine ABC transporter | B. subtilis | SEQ ID NO. 53, |
| | (permease; osmotic stress) | | 54 |
| | Glycine-betaine ABC transporter | B. licheniformis | SEQ ID NO. 113, |
| | (permease; osmotic stress) | | 114 |
| phoA | Alkaline phosphatase A (phosphate | B. subtilis | SEQ ID NO. 55, |
| | starvation) | | 56 |
| | Alkaline phosphatase (phosphate | E. coli | SEQ ID NO. 57, |
| | starvation) | | 58 |
| phoD | Phosphodiesterase/alkaline | B. subtilis | SEQ ID NO. 59, |
| | phosphatase (phosphate starvation) | | 60 |
| pstS | Phosphate ABC transporter (binding | B. subtilis | SEQ ID NO. 61, |
| | protein; phosphate starvation) | | 62 |
| | High affinity P-specific transport; | E. coli | SEQ ID NO. 63, |
| | periplasmic P binding (phosphate | | 64 |
| | starvation) | | |
| | Phosphate-binding protein | B. licheniformis | SEQ ID NO. 115, |
| | (phosphate starvation) | | 116 |
| purC | Phosphoribosylaminoimidazole | B. subtilis | SEQ ID NO. 65, |
| | succinocarboxamide synthetase | | 66 |
| | (purine synthesis) | | |
| | Phosphoribosylaminoimidazole | B. licheniformis | SEQ ID NO. 117, |
| | succinocarboxamide synthetase | | 118 |
| | (E.C. 6.3.2.6; purine synthesis) | | |
| purN | Phosphoribosylglycinamide | B. subtilis | SEQ ID NO. 67, |
| | formyltransferase (purine synthesis) | | 68 |
| | Phosphoribosylglycinamide | B. licheniformis | SEQ ID NO. 119, |
| | formyltransferase (E.C. 2.1.2.2; | | 120 |
| | purine synthesis) | | |
| pyrB | Aspartate carbamoyltransferase | B. subtilis | SEQ ID NO. 69, |
| | (pyrimidine synthesis) | | 70 |
| pyrP | Uracil permease (pyrimidine | B. subtilis | SEQ ID NO. 71, |
| | synthesis) | | 72 |
| | Uracil permease (pyrimidine | B. licheniformis | SEQ ID NO. 121, |
| | synthesis) | | 122 |
| sigB | RNA polymerase-specific general | B. subtilis | SEQ ID NO. 73, |
| | (alternative) stress sigma factor | | 74 |
| | RNA polymerase-specific general | B. licheniformis | SEQ ID NO. 123, |
| | (alternative) stress sigma factor | | 124 |
| tnrA | Pleiotropic transcriptional regulator, | B. subtilis | SEQ ID NO. 75, |
| | involved in global nitrogen | | 76 |
| | regulation (nitrogen metabolism) | | |
| trxA | Thioredoxin; heat shock IV | B. subtilis | SEQ ID NO. 77, |
| | (oxidative stress) | | 78 |
| | Thioredoxin (oxidative stress) | E. coli | SEQ ID NO. 79, |
| | | | 80 |
| | Thioredoxin; heat shock IV | B. licheniformis | SEQ ID NO. 125, |
| | (oxidative stress) | | 126 |
| ydjF | Phage-shock protein A homolog | B. subtilis | SEQ ID NO. 81, |
| (=pspA) | (sigma-W-dependent; alkaline | | 82 |
| | stress) | | |

In the sequence listing, the DNA sequences in question (in each case odd-numbered) comprise the regions coding for the particular protein and in each case approx. 200 bp upstream and downstream thereof, notwithstanding the question as to whether said regions encompass the in each case complete noncoding regions of the gene or extend into regions which already relate to the neighboring genes. In SEQ ID NO. 93 (clpP from B. licheniformis), only part of the gene is indicated; comparison with the corresponding sequence of B. subtilis (SEQ ID NO. 11) suggests that approx. 48 bp and thus 16 amino acids are missing at the 5′ end. In SEQ ID NO. 101 (dnaK from B. licheniformis), only the coding region is indicated. Moreover, seven positions toward the end of the gene in SEQ ID NO. 101, which are denoted “n” in the sequence listing, are somewhat uncertain. They recur at the amino acid level as the entry “Xaa” for the seven positions 481, 494, 495, 496, 499, 509 and 557 according to SEQ ID NO. 102. This would change, if one of these positions (e.g. 1480) were to be reviewed in the future as being part of a stop codon; the subsequent uncertain positions would then already be in the 3′-noncoding region. In any case, however, this has only a small effect on the present invention, since, as will be explained hereinbelow, preference is given rather to regions located close to the 5′ end for the preparation of probes.

The even-numbered sequence numbers represent in each case the derived amino acid sequences. They serve, for example via sequence database comparisons, to check gene function and may possibly be used in order to generate probes recognizing similar nucleic acids via backtranslation of the genetic code.

All of these genes have been described in each case separately in the prior art. They may be retrieved for the various organisms from generally accessible databases. This applies in particular to the well-characterized speciesB. subtilis and E. coli which are generally regarded as model organisms of Gram-positive and Gram-negative bacteria, respectively. The sequences set forth in the sequence listing, for example, were retrieved from the databases of Institut Pasteur, 25,28 rue du Docteur Roux, 75724 Paris CEDEX 15, France, which are accessible via the Internet at genolist.pasteur.fr/Colibri/ (for E. coli) and at genolist.pasteur.fr/SubtiList/ (for B. subtilis) (last update: Aug. 16, 2002). Other databases suitable for this are those of EMBL-European Bioinformatics Institute (EBI) in Cambridge, United Kingdom (accessible via the world wide web at ebi.ac.uk), Swiss-Prot (Geneva Bioinformatics (GeneBio) S.A., Geneva, Switzerland (accessible via the world wide web at genebio.com/sprot.html), or GenBank (National Center for Biotechnology Information NCBI, National Institutes of Health, Bethesda, Md., USA).

The genes mentioned characterize particular metabolic pathways in the relevant organisms. The skilled worker knows per se further identically regulated genes from most of these pathways, which are therefore included in the scope of protection of the present application.

Identical regulation, in particular in bacteria, can be checked by a skilled worker in a simple manner, since bacteria form a contiguous mRNA (polycistronic mRNA) from the genes which are located sequentially in an operon and are regulated via the same promoter. Said mRNA hybridizes with all probes to any of the genes reproduced on said mRNA. In eukaryotes, identically regulated genes may be identified in a different manner: to this end, the promoters which regulate expression of the genes in question must be identified by methods known per se. In this case, identically regulated genes can be recognized by the fact that they are preceded by the same promoters.

In this respect, the term “identically regulated genes in the relevant organism” refers, in the case of eukaryotes, to those genes which are regulated by the same promoters in the relevant organism and, in the case of bacteria, to those genes which are located together with the genes defined herein on a polycistronic mRNA in said bacterium.

According to the invention, a probe is a chemical compound which is capable of binding mRNA molecules via hydrogen bonds, as is the case, for example, for the interaction of the two strands of a DNA or for DNA-RNA interaction. From a chemical point of view, said compound is, for example, DNA which is more stable to hydrolysis than RNA. In addition, further molecules are known in the prior art, in particular chemically synthesized ones, which biomimetically make possible the same interaction but are more stable than DNA. Such nucleic acid-analog probes characterize preferred embodiments of the present application.

Suitable relevant organisms are in principle any plants, animals and microorganisms. Thus, for example, the application DE 19860313 A1 entitled “Verfahren zur Erkennung und Charakterisierung von Wirkstoffen gegen Pflanzen-Pathogene” [Process for recognizing and characterizing active substances against plant pathogens] reveals that there are metabolic situations in plants, in particular useful plants, which need to be observed. It is also possible to observe, for example, livestock or laboratory animals. Eukaryotic cell cultures are of quite considerable commercial interest, for example for production of monoclonal antibodies, and in particular fermentative production of food, for example via alcoholic fermentation carried out by yeasts. Bacteria are utilized, in particular, for industrial production of proteins or low molecular weight desired substances (biotransformation), for example vitamins or antibiotics.

In preferred embodiments, chips of the invention are increasingly preferably doped with at least 6, 8, 10, 12, 14, 16, 18, 20, 22, 24, 26, 28, 30, 32 or 34 of the probes mentioned, in order to record as broad a spectrum of metabolic situations as possible.

Most of the genes listed are known both in Gram-positive and Gram-negative bacteria. A few of these genes are currently known only from individual groups of organisms such as, for example, Gram-negative or Gram-positive bacteria. If, in this context, homologous representatives, i.e. representatives recognizable via hybridization, were to be found in other groups in the future, they will be included accordingly in the scope of the claims, since, according to the invention, obtaining an accurate signal via hybridization rather than determining the exact sequence of nucleotides is what matters. Thus it is possible, for example, to identify the groEL gene of a Gram-positive organism such as B. subtilis via homologous hybridization with a probe for the E. coli groL gene. Since the corresponding gene products exert the same function in both classes of organism, namely that of a chaperone, a stress situation of the relevant organism, in which a multiplicity of misfolded proteins appear, can be inferred from detection of this signal. The same probe is analogously also applicable to other species which produce a groL-similar chaperone.

This applies even more to closer related organisms such as, for example, B. subtilis andB. licheniformis. In those cases in which the sequence in question of one of these organisms or other Gram-positive bacteria is not known, it is possible to fall back on the sequences actually known in each case. Thus, examples 8 and 9 are applicable overall to various Bacillus species, and the skilled worker can be expected to produce not only one but a few alternative probes from a known sequence. Thus, via preliminary experiments and advantageously in comparison with known quantification methods (cf. examples of the present application), he achieves certain knowledge about the detectability of the gene product of interest.

It may also be expected, for example, that a probe derived from the 5′ end of the B. subtilis clpP gene (SEQ ID NO. 11) is capable of detecting the correspondingB. licheniformis gene product whose corresponding DNA sequence could be indicated in SEQ ID NO. 93 only incompletely (see above). However, according to the invention, the latter, as soon as a complete sequence is available, will be more preferred for this purpose because it will definitely be able to be used for deriving a probe having 100% sequence identity.

Other genes which are identically regulated in the relevant organism and which are on the metabolic pathways characterized in each case by said genes may serve as equivalent indicators. Thus, for example, the choice of genes characteristic for purine metabolism, purC and purN, may be considered in some respects as arbitrary. In alternative embodiments of the present invention one or more other genes which are likewise required for purine synthesis are selected, if they represent the same signal as purC and/or purN. The same applies also for the genes of the remaining relevant metabolic performances: cell wall synthesis, DNA replication, membrane transport mechanisms, carbon metabolism, citrate cycle (tricarboxylic acid cycle; TCA), respiratory chain, nitrogen metabolism, phosphate metabolism, amino acid synthesis, pyrimidine synthesis, translation, including ribosomal genes, secretion, anaerobiosis and, where appropriate, sporulation.

In a preferred embodiment, the chip targets Gram-positive bacteria, in particular B. subtilis or B. licheniformis. For this, it is recommended to dope said chip with probes selected from the group consisting of the genes acoA, ahpC, ahpF, citB, clpC, clpP, codY, cspB, des, dnaK, eno, glnR, groEL, gsiB, katA, katE, IctP, Idh, opuAB, phoA, phoD, pstS, purC, purN, pyrB, pyrP, sigB, tnrA, trxA and ydjF, and for genes regulated identically in the relevant organism from the metabolic pathways characterized in each case by said genes.

The corresponding DNA sequences are set forth in the sequence listing under the numbers SEQ ID NO. 1, 3, 5, 7, 9, 11, 15, 19, 23, 25, 29, 31, 33, 37, 43, 45, 49, 51, 53, 55, 59, 61, 65, 67, 69, 71, 73, 75, 77 and 81 for B. subtilis and SEQ ID NO. 83, 85, 87, 89, 91, 93, 95, 97, 99, 101, 103, 105, 107, 109, 111, 113, 115, 117, 119, 121, 123 and 125 for B. licheniformis. From these it is possible to derive also probes for other Gram-positive bacteria, according to the comments made above.

In a preferred embodiment, the chip targets Gram-negative bacteria, in particular E. coli or Klebsiella. For this, it is recommended to dope said chip with probes selected from the group consisting of the genes clpP, cspA, cspB, dnaK, groL, ibpA, ibpB, katE, phoA, pstS, and trxA, and for genes regulated identically in the relevant organism from the metabolic pathways characterized in each case by said genes.

The corresponding DNA sequences are set forth in the sequence listing under the numbers SEQ ID NO. 13, 17, 21, 27, 35, 39, 41, 47, 57, 63 and 79 for E. coli. From these it is possible to derive also probes for Klebsiella and other Gram-negative bacteria, according to the comments made above.

According to the statements made above, individual representatives of individual metabolic pathways may in each case be sufficient in order to give an appropriate signal. It is moreover necessary, in particular in the case of electronically evaluatable chips, to keep the total number of probes on a chip low. Chips of preferred embodiments are therefore characterized in that in each case only one of the following gene pairs or another gene identically regulated in the relevant organism from the metabolic pathway characterized in each case by one of said genes is present: ahpC or ahpF; clpC or clpP; cspA or cspB; ibpA or ibpB; IctP or Idh; phoA or phoD; purC or purN; pyrB or pyrP.

Particular biological processes, in particular production of commercially relevant compounds by microorganisms, generally involve the use of strains which are geared to the process in question rather than wild type strains. This includes, besides transformation with the genes responsible for the actual product generation, provision with selection markers or further adjustments of the metabolism up to auxotrophies. Strains of this kind possess a particular profile of requirements on growth conditions and partly possess metabolic genes which have been mutated compared with the wild-type genes. Since chips of the invention should advantageously target exactly these strains, very particularly the relevant bioprocess, these strain-specific properties must be taken into account and may be reflected in the choice of the probes in question. This applies in particular if the probes used are only parts of rather than the complete gene sequences.

In preferred embodiments, said chips are thus characterized in that the probes are probes which respond to the genes in question from the organism selected for the bioprocess, preferably those derived from genes of said organism.

This applies in particular if the organism selected for the bioprocess is a representative of unicellular eukaryotes, Gram-positive or Gram-negative bacteria.

Depending on the type of the desired product, various organisms are chosen. These mean in accordance with the invention not only the producer strains but also any organisms upstream of the production process, for example for cloning corresponding genes or for selecting suitable expression vectors.

In a preferred embodiment, the unicellular eukaryotes are protozoa or fungi, among the latter in particular yeast, very particularly Sacharomyces or Schizosaccharomyces, since these are employed as host cells in particular for the gene products of eukaryotes, particularly if said gene products are to be subjected to special modifications which can only be carried out by said strains. Said modifications include, for example, glycosylations.

The invention also comprises chips of the invention which target monitoring of the course, in particular the growth, of cell cultures of higher eukaryotes, such as rodents or humans.

In a preferred embodiment, the Gram-positive bacteria are coryneform bacteria or those of the genus Bacillus, among the latter in particular B. subtilis, B. amyloliquefaciens, B. licheniformis, B. agaradherens, B. stearothermophilus, B. lentus or B. globigii, since these are industrially particularly important producer strains. They are employed, in particular, to produce low molecular weight chemical compounds, for example vitamins or antibiotics, or to produce proteins, in particular enzymes. Particular emphasis should be made here on amylases, cellulases, lipases, oxidoreductases and proteases.

In a no less preferred embodiment, the bacteria are Gram-negative bacteria, in particular those of the genera Escherichia coli and Klebsiella. These are used both on the laboratory scale, for example for cloning and expression analysis, and on the industrial scale for producing biological desired substances.

The present application not only illustrates the above-described genes as interesting candidates for process monitoring but also makes available corresponding sequences. These are listed in the sequence listing, with the odd-numbered sequence numbers denoting in each case the genes and the even-numbered sequence numbers denoting the derived gene products.

In particular embodiments, chips of the invention are thus characterized in that at least one probe, increasingly preferably a plurality of probes, are derived from the sequences listed in the sequence listing under the numbers SEQ ID NO. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 and 125.

As stated above, the observed processes are of industrial interest which is related to further specific genes. These are, for example in the case of a protein needing to be produced, the gene for said protein and, in the case of a low molecular weight compound needing to be produced, one or more gene products which are on the synthetic pathway of the compound in question. Other genes intrinsic to the cell may also be affected, for example metabolic genes which must be increasingly produced in the course of product production, for example an oxidoreductase intrinsic to the cell, if the product is to be obtained from a reactant or an intermediate via oxidation or reduction. This is illustrated in example 5 in which a chip containing a probe for the gene of the product of interest is used. And examples 8 and 9 demonstrate which genes may be monitored additionally via a chip of the invention, if a particular enzyme is to be produced fermentatively.

Chips in a preferred embodiment are thus chips which are additionally doped with at least one probe for an additional gene, in particular one which is metabolically associated with the gene(s) additionally expressed due to the process, very particularly for any of these genes or for this gene itself.

If a polypeptide formed is itself of interest or if an endogenous activity has been altered, then chips in preferred embodiments are characterized in that the gene additionally expressed due to the process is that for a commercially usable protein, in particular an amylase, a cellulase, a lipase, an oxidoreductase or a protease, or one which is on a synthetic pathway for a low molecular weight chemical compound or which at least partially regulates said pathway.

The design of chips doped with nucleic acids is known from the prior art illustrated at the outset. Said design is based on the principle of nucleic acid hybridization of the mRNA to be detected with the probe initially introduced on the chip. Depending on the system for evaluating the signal caused by hybridization, a distinction is made between chips having an optical and chips having an electrical analytical system. According to the invention, both systems are applicable in principle.

Chips of this kind are used for monitoring the bioprocess relevant in each case: at a particular time, a sample containing the biological material to be analyzed is removed from the process; RNA, in particular mRNA is isolated from said material by methods known per se, for example with cell disruption and the use of a denaturing buffer. Advantageously, said RNA is conducted in a labeled form in a buffer over/through the chip. Hybridization (sandwich labeling) of a prepared RNA with the homologous probe provided on the chip (target nucleic acid, for example target DNA or target nucleic acid analog) results in a corresponding optically or electronically evaluatable signal. The latter is based, for example, on hybridization with a second probe or on a secondary detection reaction, for example via RT-PCR.

Since usually in each case two or more molecules of the same probe are bound to the chip, the strength of the hybridization signal across a certain region, which, in the individual case, is to be optimized, where appropriate, is proportional to the number of specific mRNA present in the sample at the time of sampling. In this way, the strength of the signal is a direct measure of the activity of the gene in question at the time of sampling.

In this connection, the time between sampling and measurement should be kept as short as possible, for example by way of substantially automated sampling, work-up of the samples and conduction thereof over/through the sensor.

Limiting the usability of a probe is thus in each case the extent of homology between the probe provided and the mRNA which is to be recognized by way of hybridization. Ultimately, the extent of hybridization of the probe with the mRNA to be detected (see above) decides its usability as a probe and, in individual cases, has to be optimized experimentally and/or taken into account by way of adjusting signal evaluation. Under the conditions determined by the construction of the measuring apparatus and other influences, a hybridization must take place which can specifically be attributed only to the gene of interest, is sufficiently strong in order to give a positive signal and, on the other hand, is not too strong so that, after the signal has been generated, the mRNA diffuses off in order to render the binding site empty for the next molecule, or to enable the signal to decay.

It is, however, necessary to estimate the extent of homology between the genes in question prior to using chips of the invention for an organism of interest; to anchor, if the affinity of the mRNAs to be detected to the initially introduced probes is not sufficient, those probes by way of the same genes of the invention from closer related species on the chip and to carry out calibration measurements in order to obtain reliable information as to which signal strength corresponds to which concentration of special mRNA.

In order to achieve optimal hybridization, chips of preferred embodiments are characterized in that one probe, preferably a plurality of probes, are provided single-strandedly, in the form of the codogenic strand, since the latter hybridizes with the coding strand of the DNA, and with the corresponding mRNA.

Advantageously, it should be possible to use chips of the invention multiple times, in particular during a single observed process in the course of which continuous monitoring is desirable. In order to increase for this purpose the stability of chips of the invention, in particular to nucleic acid-hydrolyzing enzymes, chips of the invention are characterized in that one probe, preferably a plurality of probes are made available in the form of a DNA, preferably of a nucleic acid analog. A DNA is per se less sensitive to hydrolysis than, for example, an RNA. In contrast, however, preference is given to analogs which are difficult to hydrolyze and in which, for example, the phosphate of the sugar-phosphate backbone has been replaced. Compounds of this kind are known in the prior art. The corresponding probes would have to be synthesized according to the example of the sequence listing related to this application.

Detecting an mRNA often does not require hybridization over the entire length of the sequence. The specific probes therefore need to comprise the gene region which is actually to be detected as mRNA rather than that which is transcribed into mRNA. Advantageous for this purpose is a selection of a region which is close to the 5′ end of the mRNA, since this region is transcribed first into mRNA and is thus the first to be detected after activation of the gene. This fits in with a detection close to real time.

In a preferred embodiment, chips of the invention are thus characterized in that one probe, preferably a plurality of probes, comprise gene regions which are transcribed into mRNA by the organism to be studied, in particular the gene regions which are close to the 5′ end of said mRNA.

mRNA molecules often have a secondary structure which is based on the hybridization of individual mRNA regions with other regions of the same molecule. Thus, for example, loop or stem-loop structures arise. Such regions, however, usually hybridize less readily with other nucleic acid molecules, even if those are homologous.

In preferred embodiments, chips of the invention are therefore characterized in that one probe, preferably a plurality of probes, respond to fragments of the nucleic acids in question, in particular to those which have a low degree of secondary folding in the mRNA in question, based on the particular total mRNA.

The probes employed in the detection reaction need to comprise only part of the mRNA to be detected, as long as the signal obtainable thereby is still specific enough. This specificity determines the lower limit of the length of the probes in question.

Suitable probes are normally identified with the aid of specialized software. Examples of such software are the program Array Designer from Premier Biosoft International, USA, and the program Primer 3 which is freely accessible on world wide web at genome.wi.mit.edu/cgi-bin/primer/primer3.cgi. In addition to the secondary structures already mentioned, these software programs also take into account, for example, predefined probe lengths and melting temperatures.

Preferably, a chip of the invention is thus characterized in that one probe, preferably a plurality of probes, have a length of less than 200 nucleotides, and increasingly preferably of less than 150, 120, 100, 80, or from 20 to 60, 30 to 50, and particularly preferably from 45 to 55, nucleotides. In the examples of the present application, even those probes whose length was in each case only 20 bases have proved suitable.

Chips of the invention are preferably characterized in that binding of the mRNA to the probe in question triggers an electrical signal.

The previously mentioned article J. Wang (Acc. Chem. Res.; ISSN 0001-4842; Rec. Sep. 12, 2001, S. A-F) discusses the advantages of an electrically evaluatable system compared with an optical system. Reference is also made to various embodiments of such sensors, which have been developed in the prior art.

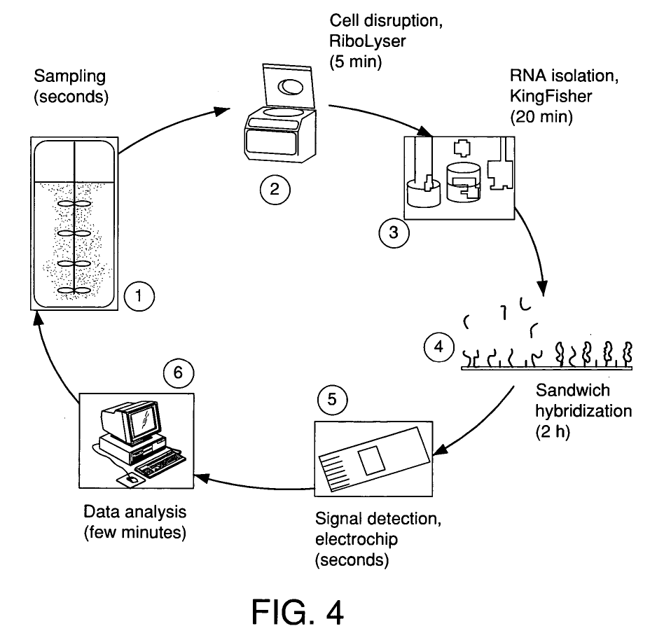

Thus, the time from sampling to measuring the signal is currently approximately 24 h for optically evaluatable chips. The time needed with the aid of an electrical system is at the moment approx. 2 h (cf. FIG. 4). In contrast, the number of simultaneously analyzable samples in the case of electrically evaluatable chips is currently limited to a maximum of 12 probes, with a rapid development suggesting, however, that it will soon be possible to provide more analysis places on a chip. Limiting this are the electronic evaluation units for the various signals.

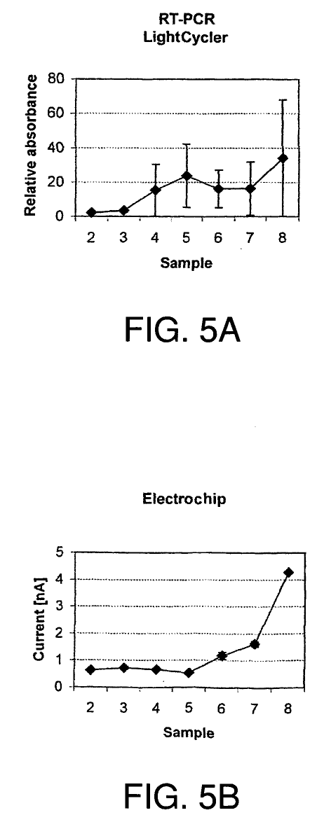

One example of mRNA quantification methods established in the prior art is RT-PCT. This is described in the article “Quantification of Bacterial mRNA by One-Step RT-PCR using the LightCycler System” (2003) by S. Tobisch, T. Koburger, B. Jürgen, S. Leja, M. Hecker and T. Schweder in BIOCHEMICA, volume 3, pages 5 to 8. In contrast, detection via electro chips has another advantage, illustrated in example 4 and FIG. 5, namely higher reliability of the data, since the latter have, as demonstrated there, distinctly smaller fluctuation ranges compared with RT-PCR.

The preparation of corresponding electronically evaluatable chips is described, for example, in the patent applications WO 00/62048 A2, WO 00/67026 A1 and WO 02/41992 whose entire disclosure content is incorporated into the present application.

The function of electrically readable chips of a particularly preferred embodiment may be described as follows: the gene-specific probes are bound covalently in a manner known per se to magnetic beads which are located in chambers designed therefor of the chips. Specific hybridization of the appropriate mRNA to the particular beads occurs in this hybridization chamber whose temperature can be controlled and which can be flushed with the solutions in question. The beads are kept in said chamber by means of a magnet. After hybridization of the RNA samples to the beads-bound DNA probes, a washing step is carried out to remove the unbound RNA so that only specific hybrids, bound to the magnetic beads, are still present in the incubation chamber.

After washing, a detection probe which is labeled by way of a biotin-extravidin-bound alkaline phosphatase is introduced into the incubation chamber. Said probe binds to a second free region of the hybridized mRNA. This hybrid is then washed again and incubated with the substrate of said alkaline phosphatase, para-aminophenol phosphate (pAPP). The enzymic reaction in the incubation chamber results in the release of the redox-active product para-aminophenol (pAP). The latter is then passed over the Red/Ox electrode on the electrical chip, and the signal is sent to a potentiostat.

A system-specific software (e.g. MCDDE32) reads the obtained data and the results may be evaluated and depicted with the aid of a further program (e.g. Origin), using a computer.

Said process can be varied, of course, with regard to both technical design of the chips and evaluation. Thus, for example, the detection reaction may be carried out by way of another reaction but preferably by way of a redox reaction, due to the electrical measuring principle

One achievement of the present invention consists of having identified process-particular genes and having made accessible said genes to analysis via correspondingly designed biochips. Besides time saved and higher accuracy, the advantage of chips compared with conventional detection methods is the fact that providing a plurality of probes on a support enables the activities of a plurality of different genes to be detected in the same sample at the same time.

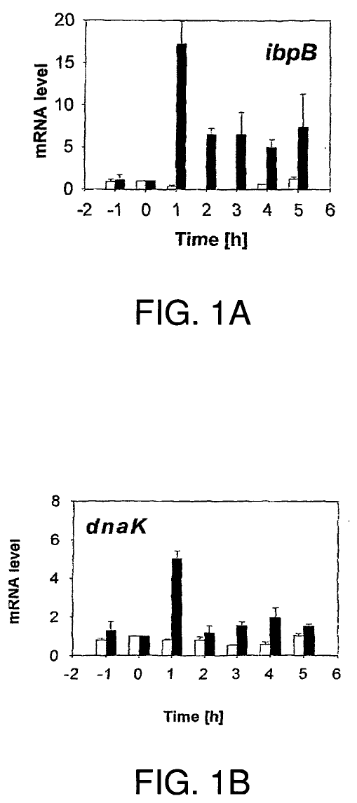

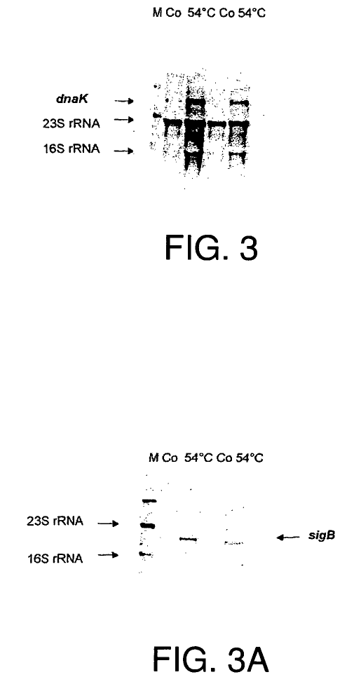

Thus, the present invention provides the use of corresponding probes selected so as to produce a picture, representative for most fermentation courses, about various physiological states of the Gram-negative and Gram-positive bacteria in question. This applies in particular to those of the species E. coli (cf. example 1 and FIG. 1),B. subtilis (cf. example 2 and FIG. 2) and B. licheniformis (cf. example 3 and FIG. 3). The probes described herein which are specific for the genes ibpB, dnaK, acoA and sigB, may accordingly be bound in a manner known per se to corresponding chips and used for analyzing bioprocesses. In example 4 and example 7, glucose starvation-indicating acoA-mRNA of B. subtilis and B. licheniformis , respectively, in example 6 the phosphate deficiency-indicating B. licheniformis pstS gene product and in example 5 aprE, an mRNA for a gene product of interest, are detected via chips. Examples 8 and 9 describe electrically evaluatable chips containing eleven probes at the same time, with seven probes reflecting the general metabolic situation, in each case one probe targeting the product of interest and in each case three further genes monitoring those metabolic aspects which are associated with the product of interest (cf. tables 5 and 6). Said probes here are, in the case of the product protease, those for genes of nitrogen metabolism and, in the case of the product amylase, those for genes of carbohydrate metabolism. Analogously, it would be possible to observe, for example during a biotransformation, the gene activities required for chemical conversion of the substrates in question.

A separate subject matter of the invention is thus the use of nucleic-acid probes or nucleic acid-analog probes for at least four of the following genes: acoA, ahpC, ahpF, citB, clpC, clpP, codY, cspA, cspB, des, dnaK, eno, glnR, groEL, groL, gsiB, ibpA, ibpB, katA, katE, IctP, Idh, opuAB, phoA, phoD, pstS, purC, purN, pyrB, pyrP, sigB, tnrA, trxA and ydjF, and for genes identically regulated in the relevant organism from the metabolic pathways characterized in each case by said genes, bound to a chip described above, for determining the physiological state of an organism undergoing a biological process.

Preference is given in this context to those uses which are characterized in that at least one probe, increasingly preferably a plurality of probes, are derived from the sequences listed in the sequence listing under the numbers SEQ ID NO. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 and 125.

Further preferred embodiments of this subject matter and explanations thereof arise from the comments made above regarding the chips of the invention.

Another separate subject matter of the invention comprises, according to the comments made above, processes for determining the physiological state of an organism undergoing a biological process by using an above-described chip of the invention.

Preferably, a process of the invention is characterized in that the organism selected for the bioprocess is a representative of unicellular eukaryotes, Gram-positive or Gram-negative bacteria.

Preferably, a process of the invention is characterized in that the unicellular eukaryotes are protozoa or fungi, among the latter in particular yeast, very particularly Sacharomyces or Schizosaccharomyces.

Preferably, a process of the invention is characterized in that the Gram-positive bacteria are coryneform bacteria or those of the genus Bacillus, among the latter in particular B. subtilis, B. amyloliquefaciens, B. licheniformis, B. agaradherens, B. stearothermophilus, B. lentus or B. globigii, among these particularly preferably those with probes derived from the B. subtilis or B. licheniformis sequences set forth in the sequence listing (SEQ ID NO. 1, 3, 5, 7, 9, 11, 15, 19, 23, 25, 29, 31, 33, 37, 43, 45, 49, 51, 53, 55, 59, 61, 65, 67, 69, 71, 73, 75, 77, 81, 83, 85, 87, 89, 91, 93, 95, 97, 99, 101, 103, 105, 107, 109, 111, 113, 115, 117, 119, 121, 123 and/or 125).

Preference is given to using for this purpose probes which are derived from the genes in question of related species, if possible, particularly preferably of the particular organism itself.

This means, in the case ofB. subtilis and B. licheniformis, those processes which are characterized in that the species is B. subtilis or B. licheniformis, with, in the case of B. subtilis, the probes being derived from the B. subtilis sequences set forth in the sequence listing (SEQ ID NO. 1, 3, 5, 7, 9, 11, 15, 19, 23, 25, 29, 31, 33, 37, 43, 45, 49, 51, 53, 55, 59, 61, 65, 67, 69, 71, 73, 75, 77 and/or 81), and, in the case of B. licheniformis, the probes being derived from the B. licheniformis sequences set forth in the sequence listing (SEQ ID NO. 83, 85, 87, 89, 91, 93, 95, 97, 99, 101, 103, 105, 107, 109, 111, 113, 115, 117, 119, 121, 123 and/or 125).

Preferably, a process of the invention is characterized in that the Gram-negative bacteria are those of the genera Escherichia coli or Klebsiella, preferably with probes derived from the E. coli sequences set forth in the sequence listing (SEQ ID NO. 13, 17, 21, 27, 35, 39, 41, 47, 57, 63 and/or 79).

In this context, preference is given to those processes which are characterized in that the species is E. coli, due to their great importance, in particular to experiments on the laboratory scale.

Preferably, a process of the invention is characterized in that the physiological state is determined at various points in time of the same process, optionally with the use of a plurality of identically constructed chips.

Preferably, a process of the invention is characterized in that the process is a fermentation, in particular the fermentative preparation of a commercially usable product, particularly preferably the preparation of a protein or of a low molecular weight chemical compound.

A separate subject matter of the invention is the use of a chip of the invention for determining the physiological state of an organism undergoing a biological process.

Preferred embodiments of such uses as well as of the use of the gene probes defined in the present invention arise from the previously made comments.

Preferably, a use of the invention is characterized in that the organism selected for the bioprocess is a representative of unicellular eukaryotes, Gram-positive or Gram-negative bacteria.

Preferably, a use of the invention is characterized in that the unicellular eukaryotes are protozoa or fungi, among the latter in particular yeast, very particularly Sacharomyces or Schizosaccharomyces.

Preferably, a use of the invention is characterized in that the Gram-positive bacteria are coryneform bacteria or those of the genus Bacillus, among the latter in particular B. subtilis, B. amyloliquefaciens, B. licheniformis, B. agaradherens, B. stearothermophilus, B. lentus or B. globigii, among these particularly preferably those with probes derived from the B. subtilis or B. licheniformis sequences set forth in the sequence listing (SEQ ID NO. 1, 3, 5, 7, 9, 11, 15, 19, 23, 25, 29, 31, 33, 37, 43, 45, 49, 51, 53, 55, 59, 61, 65, 67, 69, 71, 73, 75, 77, 81, 83, 85, 87, 89, 91, 93, 95, 97, 99, 101, 103, 105, 107, 109, 111, 113, 115, 117, 119, 121, 123 and/or 125).

If the observed organisms are B. subtilis or B. licheniformis, preference is given in this context to those uses which are characterized in that in the case of B. subtilis the probes are derived from the B. subtilis sequences set forth in the sequence listing (SEQ ID NO. 1, 3, 5, 7, 9, 11, 15, 19, 23, 25, 29, 31, 33, 37, 43, 45, 49, 51, 53, 55, 59, 61, 65, 67, 69, 71, 73, 75, 77 and/or 81), and, in the case of B. licheniformis, the probes are derived from the B. licheniformis sequences set forth in the sequence listing (SEQ ID NO. 83, 85, 87, 89, 91, 93, 95, 97, 99, 101, 103, 105, 107, 109, 111, 113, 115, 117, 119, 121, 123 and/or 125).

Preferably, a use of the invention is characterized in that the Gram-negative bacteria are those of the genera Escherichia coli or Klebsiella, preferably with probes derived from the E. coli sequences set forth in the sequence listing (SEQ ID NO. 13, 17, 21, 27, 35, 39, 41, 47, 57, 63 and/or 79).

Preference is given in this context to those uses which are characterized in that the species is E. coli.

Preferably, a use of the invention is characterized in that the physiological state is determined at various points in time of the same process, optionally with the use of a plurality of identically constructed chips.

Preferably, a use of the invention is characterized in that the process is a fermentation, in particular the fermentative preparation of a commercially usable product, particularly preferably the preparation of a protein or of a low molecular weight chemical compound.

In connection with the present application, most of the genes usable according to the invention and listed in table 2 were also isolated from Bacillus licheniformis DSM 13 and sequenced. This strain is generally obtainable via the Deutsche Sammlung von Mikroorganismen und Zellkulturen GmbH, Mascheroder Weg 1b, 38124 Braunschweig, Germany. Said strain has the deposition number ATCC 14580 with the American Type Culture Collection, 10801 University Boulevard, Manassas. Va. 20110-2209, USA. The sequencing reactions were carried out by means of well-known processes.

The DNA sequences and amino acid sequences, not yet described in the prior art, of these enzymes which are, however, known in principle are listed in the sequence listing of the present application. They are again compiled, together with the corresponding numbers of the sequence listing, in table 3 below. Additionally listed are the homologies to B. subtilis determined in each case, unless another, still more homologous sequence was known; in this case, the latter is likewise indicated. B. subtilis is one of the closest relatives of B. licheniformis so that, according to expectation, no even more similar DNA and amino acid sequences should be known. This is the basis of the scope of protection indicated in each case in the corresponding claims.

Table 3: Genes of the invention and derived proteins of B. licheniformis

Indicated are in each case the homologies to the corresponding sequences of B. subtilis as one of the next most similar organisms, unless an even more similar sequence was known (indicated).

| | | DNA | Homology to B. subtilis at | |
|---|---|---|---|---|
| | | sequence and | DNA level | |
| | | amino acid | (coding | Amino acid |
| Gene | Function | sequence | region) [%] | level [%] |
| acoA | Acetoin dehydrogenase E1 | SEQ ID NO. | 81 | 70 |
| | component (TPP-dependent α | 83, 84 | | |
| | subunit; E.C.1.2.4.—; glucose | | | |
| | limitation) | | | |
| ahpC | Alkyl hydroperoxide reductase | SEQ ID NO. | 87 | 91 |
| | (E.C. 1.6.4.—; small subunit; | 85, 86 | | |
| | general stress; stationary phase) | | | |
| ahpF | Alkyl hydroperoxide reductase | SEQ ID NO. | 83 | 88 |
| | (large subunit)/NADH | 87, 88 | | |
| | dehydrogenase (E.C. 1.6.99.3; | | | |
| | general stress; stationary phase) | | | |
| citB | Aconitate hydratase (EC 4.2.1.3; | SEQ ID NO. | 89 | 89 |
| | citrate cycle-active) | 89, 90 | | |
| clpC | Class III stress response-related | SEQ ID NO. | 80 | 91 |
| | ATPase | 91, 92 | | |
| clpP | ATP-dependent protease, | SEQ ID NO. | 82 | 92 |
| | proteolytic subunit (class III heat | 93, 94 | | |
| | shock protein; E.C. 3.4.21.92) | (incomplete) | | |
| codY | Pleiotropic transcriptional | SEQ ID NO. | 84 | 88 |
| | repressor (nitrogen metabolism) | 95, 96 | | |
| cspB | Major cold shock protein | SEQ ID NO. | 95 | 96 |
| | (stationary phase) | 97, 98 | | |
| des | Fatty acid desaturase (E.C. | SEQ ID NO. | 84% from B. anthracis; | 69% from B. anthracis; |
| | 1.14.99.—; formation of | 99, 100 | 80% from | 69% from |
| | unsaturated fatty acids) | | B. subtilis | B. subtilis |
| dnaK | Class I heat shock protein | SEQ ID NO. | 84 | 90 |
| | (molecular chaperone) | 101, 102 (7 | | |
| | | positions | | |
| | | toward the end | | |
| | | of the gene | | |
| | | uncertain) | | |
| eno | Enolase (E.C. 4.1.2.11; glucose | SEQ ID NO. | 90 | 96 |
| | starvation) | 103, 104 | | |
| glnR | Transcriptional repressor of the | SEQ ID NO, | 87 | 79 |
| | glutamine synthetase gene | 105, 106 | | |
| | (nitrogen metabolism) | | | |
| groEL | Class I heat shock protein | SEQ ID NO. | 86 | 92 |
| | (chaperonin) | 107, 108 | | |
| katA | Catalase (E.C. 1.11.1.6; | SEQ ID NO. | 82 | 86 |
| | oxidative stress) | 109, 110 | | |
| katE | Catalase (E.C. 1.11.1.6; | SEQ ID NO. | 81 | 70 |
| | oxidative stress) | 111, 112 | | |
| opuAB | Glycine-betaine ABC transporter | SEQ ID NO. | 81 | 78 |
| | (permease; osmotic stress) | 113, 114 | | |
| pstS | Phosphate-binding protein | SEQ ID NO. | 80 | 78 |
| | (phosphate starvation) | 115, 116 | | |
| purC | Phosphoribosylaminoimidazole | SEQ ID NO. | 85 | 86 |
| | succinocarboxamide synthetase | 117, 118 | | |
| | (E.C. 6.3.2.6; purine synthesis) | | | |
| purN | Phosphoribosylglycinamide | SEQ ID NO. | 80 | 72 |
| | formyltransferase (E.C. 2.1.2.2; | 119, 120 | | |
| | purine synthesis) | | | |
| pyrP | Uracil permease (pyrimidine | SEQ ID NO. | 84 | 65 |
| | synthesis) | 121, 122 | | |
| sigB | RNA polymerase-specific | SEQ ID NO. | 96 | 81 |
| | general (alternative) stress sigma | 123, 124 | | |
| | factor | | | |
| trxA | Thioredoxin; heat shock IV | SEQ ID NO. | 89 | 97 |
| | (oxidative stress) | 125, 126 | | |

Each of these proteins is thus, together with the in each case corresponding nucleotide sequence, including the homology regions in question, a separate subject matter of the present application. The particular biochemical functions exerted by said proteins are likewise indicated in table 3 and can be checked on the basis of the database information given above. According to the invention, they are regarded as enzymes which exert the biochemical functions corresponding to those functions which are exerted in vivo in B. licheniformis by the enzymes exactly indicated in the sequence listing.

These subject matters are listed below; the particular homologies, given in percent identity, may be checked by means of an appropriate computer algorithm, for example by means of the program Vector NTI® Suite, from InforMax, Bethesda, USA; this includes all integers and any intermediate fractions:

Alpha subunit of the acetoin dehydrogenase E1 component (AcoA; E.C.1.2.4.-) having an amino acid sequence which is at least 74%, and increasingly preferably 75, 76, 77, 78, 79, 80, 81, 82, 83, 84, 85, 86, 87, 88, 89, 90, 91, 92, 93, 94, 95, 96, 97, 98, 99 and 100%, identical to the amino acid sequence set forth in SEQ ID NO. 84; in connection with a nucleic acid (acoA), encoding an alpha subunit of the acetoin dehydrogenase E1 component (AcoA; E.C.1.2.4.-) and which has a nucleotide sequence which is at least 85%, and increasingly preferably 86, 87, 88, 89, 90, 91, 92, 93, 94, 95, 96, 97, 98, 99 and 100%, identical to the nucleotide sequence set forth in SEQ ID NO. 83, in particular across the subregion corresponding to nucleotide positions 201 to 872 according to SEQ ID NO. 83.

Small subunit of alkyl hydroperoxide reductase (AhpC; E.C.1.6.4.-), having an amino acid sequence which is at least 95%, and increasingly preferably 96, 97, 98, 99 and 100%, identical to the amino acid sequence set forth in SEQ ID NO. 86; in connection with a nucleic acid (ahpC), encoding a small subunit of alkyl hydroperoxide reductase (AhpC; E.C.1.6.4-) and having a nucleotide sequence which is at least 91%, and increasingly preferably 92, 93, 94, 95, 96, 97, 98, 99 and 100%, identical to the nucleotide sequence set forth in SEQ ID NO. 85, in particular across the subregion corresponding to nucleotide positions 201 to 764 according to SEQ ID NO. 85.

Large subunit of alkyl hydroperoxide reductase/NADH dehydrogenase (AhpF; E.C.1.6.99.3), having an amino acid sequence which is at least 92%, and increasingly preferably 93, 94, 95, 96, 97, 97.5, 98, 99 and 100%, identical to the amino acid sequence set forth in SEQ ID NO. 88; in connection with a nucleic acid (ahpF), encoding a large subunit of alkyl hydroperoxide reductase/NADH dehydrogenase (AhpF; E.C.1.6.99.3) and having a nucleotide sequence which is at least 87%, and increasingly preferably 88, 89, 90, 91, 92, 93, 94, 95, 96, 97, 98, 99 and 100%, identical to the nucleotide sequence set forth in SEQ ID NO. 87, in particular across the subregion corresponding to nucleotide positions 201 to 1730 according to SEQ ID NO. 87.

Aconitase hydratase (CitB; E.C.4.2.1.3), having an amino acid sequence which is at least 93%, and increasingly preferably 94, 95, 96, 97, 97.5, 98, 99 and 100%, identical to the amino acid sequence set forth in SEQ ID NO. 90; in connection with a nucleic acid (citB), encoding an aconitase hydratase (CitB; E.C.4.2.1.3) and having a nucleotide sequence which is at least 93%, and increasingly preferably 92, 93, 94, 95, 96, 97, 97.5, 98, 99 and 100%, identical to the nucleotide sequence set forth in SEQ ID NO. 89, in particular across the subregion corresponding to nucleotide positions 201 to 2927 according to SEQ ID NO. 89.

Class III stress response-related ATPase (ClpC), having an amino acid sequence which is at least 95%, and increasingly preferably 96, 96.5, 97, 98, 99 and 100%, identical to the amino acid sequence set forth in SEQ ID NO. 92; in connection with a nucleic acid (clpC), encoding a class III stress response-related ATPase (ClPC) and having a nucleotide sequence which is at least 84%, and increasingly preferably 85, 86, 87, 88, 89, 90, 91, 92, 93, 94, 95, 96, 97, 98, 99 and 100%, identical to the nucleotide sequence set forth in SEQ ID NO. 91, in particular across the subregion corresponding to nucleotide positions 201 to 2633 according to SEQ ID NO. 91.

Proteolytic subunit of the ATP-dependent protease (class III heat shock protein; E.C. 3.4.21.92; ClpP), having an amino acid sequence which is at least 97%, and increasingly preferably 98, 98.5, 99, 99.5 and 100%, identical to the amino acid sequence set forth in SEQ ID NO. 94; in connection with a nucleic acid (clpP), encoding a proteolytic subunit of the ATP-dependent protease (class III heat shock protein; E.C. 3.4.21.92; ClpP) and having a nucleotide sequence which is at least 86%, and increasingly preferably 87, 88, 89, 90, 91, 92, 93, 94, 95, 96, 97, 98, 99 and 100%, identical to the nucleotide sequence set forth in SEQ ID NO. 93, in particular across the subregion corresponding to nucleotide positions 1 to 549 according to SEQ ID NO. 93.

Transcriptional pleiotropic repressor (CodY), having an amino acid sequence which is at least 92%, and increasingly preferably 93, 94, 95, 96, 97, 98, 99 and 100%, identical to the amino acid sequence set forth in SEQ ID NO. 96; in connection with a nucleic acid (codY), encoding a transcriptional pleiotropic repressor (CodY) and having a nucleotide sequence which is at least 88%, and increasingly preferably 89, 90, 91, 92, 93, 94, 95, 96, 97, 98, 99 and 100%, identical to the nucleotide sequence set forth in SEQ ID NO. 95, in particular across the subregion corresponding to nucleotide positions 201 to 980 according to SEQ ID NO. 95.

Major cold shock protein (CspB), having an amino acid sequence which is at least 98%, and increasingly preferably 98.5, 99, 99.5 and 100%, identical to the amino acid sequence set forth in SEQ ID NO. 98; in connection with a nucleic acid (cspB), encoding a major cold shock protein (CspB) and having a nucleotide sequence which is at least 97%, and increasingly preferably 98, 98.5, 99, 99.5 and 100%, identical to the nucleotide sequence set forth in SEQ ID NO. 97, in particular across the subregion corresponding to nucleotide positions 201 to 401 according to SEQ ID NO. 97.

Fatty acid desaturase (Des; E.C. 1.14.99.-) having an amino acid sequence which is at least 73%, and increasingly preferably 74, 75, 76, 77, 78, 79, 80, 81, 82, 83, 84, 85, 86, 87, 88, 89, 90, 91, 92, 93, 94, 95, 96, 97, 98, 99 and 100%, identical to the amino acid sequence set forth in SEQ ID NO. 100; in connection with a nucleic acid (des), encoding a fatty acid desaturase (Des; E.C. 1.14.99.-) and having a nucleotide sequence which is at least 88%, and increasingly preferably 89, 90, 91, 92, 93, 94, 95, 96, 97, 98, 99 and 100%, identical to the nucleotide sequence set forth in SEQ ID NO. 99, in particular across the subregion corresponding to nucleotide positions 201 to 1229 according to SEQ ID NO. 99.

Class I heat shock protein (molecular chaperone; DnaK), having an amino acid sequence which is at least 94%, and increasingly preferably 95, 96, 97, 98, 99 and 100%, identical to the amino acid sequence set forth in SEQ ID NO. 102, in particular across the subregion corresponding to amino acid positions 1 to 480 according to SEQ ID NO. 102; in connection with a nucleic acid (dnaK), encoding a class I heat shock protein (molecular chaperone; DnaK) and having a nucleotide sequence which is at least 88%, and increasingly preferably 89, 90, 91, 92, 93, 94, 95, 96, 97, 98, 99 and 100%, identical to the nucleotide sequence set forth in SEQ ID NO. 101, in particular across the subregion corresponding to nucleotide positions 1 to 1440 according to SEQ ID NO. 101.

Enolase (Eno; E.C. 4.1.2.11), having an amino acid sequence which is at least 98%, and increasingly preferably 98.5, 99, 99.5 and 100%, identical to the amino acid sequence set forth in SEQ ID NO. 104; in connection with a nucleic acid (eno), encoding an enolase (Eno; E.C. 4.1.2.11) and having a nucleotide sequence which is at least 94%, and increasingly preferably 95, 96, 97, 98, 99 and 100%, identical to the nucleotide sequence set forth in SEQ ID NO. 103, in particular across the subregion corresponding to nucleotide positions 201 to 1493 according to SEQ ID NO. 103.

Transcriptional repressor of the glutamine synthetase gene (GlnR), having an amino acid sequence which is at least 83%, and increasingly preferably 84, 85, 86, 87, 88, 89, 90, 91, 92, 93, 94, 95, 96, 97, 98, 99 and 100%, identical to the amino acid sequence set forth in SEQ ID NO. 106; in connection with a nucleic acid (glnR), encoding a transcriptional repressor of the glutamine synthetase gene (GlnR) and having a nucleotide sequence which is at least 91%, and increasingly preferably 92, 93, 94, 95, 96, 97, 98, 99 and 100%, identical to the nucleotide sequence set forth in SEQ ID NO. 105, in particular across the subregion corresponding to nucleotide positions 201 to 608 according to SEQ ID NO. 105.

Class I heat shock protein (chaperonin; GroEL), having an amino acid sequence which is at least 96%, and increasingly preferably 97, 97.5, 98, 98.5, 99, 99.5 and 100%, identical to the amino acid sequence set forth in SEQ ID NO. 108; in connection with a nucleic acid (groEL), encoding a class I heat shock protein (chaperonin; GroEL) and having a nucleotide sequence which is at least 90%, and increasingly preferably 91, 92, 93, 94, 95, 96, 97, 98, 99 and 100%, identical to the nucleotide sequence set forth in SEQ ID NO. 107, in particular across the subregion corresponding to nucleotide positions 201 to 1835 according to SEQ ID NO. 107.

Catalase (KatA; E.C. 1.11.1.6), having an amino acid sequence which is at least 90%, and increasingly preferably 91, 92, 93, 94, 95, 96, 97, 98, 99 and 100%, identical to the amino acid sequence set forth in SEQ ID NO. 110; in connection with a nucleic acid (katA), encoding a catalase (KatA; E.C. 1.11.1.6) and having a nucleotide sequence which is at least 86%, and increasingly preferably 87, 88, 89, 90, 91, 92, 93, 94, 95, 96, 97, 98, 99 and 100%, identical to the nucleotide sequence set forth in SEQ ID NO. 109, in particular across the subregion corresponding to nucleotide positions 201 to 1661 according to SEQ ID NO. 109.

Catalase (KatE; E.C. 1.11.1.6), having an amino acid sequence which is at least 74%, and increasingly preferably 75, 76, 77, 78, 79, 80, 81, 82, 83, 84, 85, 86, 87, 88, 89, 90, 91, 92, 93, 94, 95, 96, 97, 98, 99 and 100%, identical to the amino acid sequence set forth in SEQ ID NO. 112; in connection with a nucleic acid (katE), encoding a catalase (KatE; E.C. 1.11.1.6) and having a nucleotide sequence which is at least 85%, and increasingly preferably 86, 87, 88, 89, 90, 91, 92, 93, 94, 95, 96, 97, 98, 99 and 100%, identical to the nucleotide sequence set forth in SEQ ID NO. 111, in particular across the subregion corresponding to nucleotide positions 201 to 1661 according to SEQ ID NO. 111.

Glycine-betaine ABC transporter (OpuAB), having an amino acid sequence which is at least 82%, and increasingly preferably 83, 84, 85, 86, 87, 88, 89, 90, 91, 92, 92.5, 93, 94, 95, 96, 97, 98, 99 and 100%, identical to the amino acid sequence set forth in SEQ ID NO. 114; in connection with a nucleic acid (opuAB), encoding a glycine-betaine ABC transporter (OpuAB) and having a nucleotide sequence which is at least 85%, and increasingly preferably 86, 87, 88, 89, 90, 91, 92, 93, 94, 95, 96, 97, 98, 99 and 100%, identical to the nucleotide sequence set forth in SEQ ID NO. 113, in particular across the subregion corresponding to nucleotide positions 201 to 1055 according to SEQ ID NO. 113.

Phosphate-binding protein (PstS), having an amino acid sequence which is at least 82%, and increasingly preferably 83, 84, 85, 86, 87, 88, 89, 90, 91, 92, 93, 94, 95, 96, 97, 98, 99 and 100%, identical to the amino acid sequence set forth in SEQ ID NO. 116; in connection with a nucleic acid (pstS), encoding a phosphate-binding protein (PstS) and having a nucleotide sequence which is at least 84%, and increasingly preferably 85, 86, 87, 88, 89, 90, 91, 92, 93, 94, 95, 96, 97, 98, 99 and 100%, identical to the nucleotide sequence set forth in SEQ ID NO. 115, in particular across the subregion corresponding to nucleotide positions 201 to 1118 according to SEQ ID NO. 115.

Phosphoribosylaminoimidazole succinocarboxamide synthetase (PurC; E.C. 6.3.2.6), having an amino acid sequence which is at least 90%, and increasingly preferably 91, 92, 93, 94, 95, 96, 97, 98, 99 and 100%, identical to the amino acid sequence set forth in SEQ ID NO. 118; in connection with a nucleic acid (purC), encoding a phosphoribosylaminoimidazole succinocarboxamide synthetase (PurC; E.C. 6.3.2.6) and having a nucleotide sequence which is at least 89%, and increasingly preferably 90, 91, 92, 93, 94, 95, 96, 97, 98, 99 and 100%, identical to the nucleotide sequence set forth in SEQ ID NO. 117, in particular across the subregion corresponding to nucleotide positions 201 to 917 according to SEQ ID NO. 117.

Phosphoribosylglycinamide formyltransferase (PurN; E.C. 2.1.2.2), having an amino acid sequence which is at least 76%, and increasingly preferably 77, 78, 79, 80, 81, 82, 83, 84, 85, 86, 87, 88, 89, 90, 91, 92, 93, 94, 95, 96, 97, 98, 99 and 100%, identical to the amino acid sequence set forth in SEQ ID NO. 120; in connection with a nucleic acid (purN), encoding a phosphoribosylglycinamide formyltransferase (PurN; E.C. 2.1.2.2) and having a nucleotide sequence which is at least 84%, and increasingly preferably 85, 86, 87, 88, 89, 90, 91, 92, 93, 94, 95, 96, 97, 98, 99 and 100%, identical to the nucleotide sequence set forth in SEQ ID NO. 119, in particular across the subregion corresponding to nucleotide positions 201 to 788 according to SEQ ID NO. 119.

Uracil permease (PyrP), having an amino acid sequence which is at least 69%, and increasingly preferably 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100%, identical to the amino acid sequence set forth in SEQ ID NO. 122; in connection with a nucleic acid (pyrP), encoding a uracil permease (PyrP) and having a nucleotide sequence which is at least 89%, and increasingly preferably 90, 91, 92, 93, 94, 95, 96, 97, 98, 99 and 100%, identical to the nucleotide sequence set forth in SEQ ID NO. 121, in particular across the subregion corresponding to nucleotide positions 201 to 1505 according to SEQ ID NO. 121.

RNA polymerase-specific general (alternative) stress sigma factor (SigB), having an amino acid sequence which is at least 85%, and increasingly preferably 86, 87, 88, 89, 90, 91, 92, 93, 94, 95, 96, 97, 98, 99 and 100%, identical to the amino acid sequence set forth in SEQ ID NO. 124; in connection with a nucleic acid (sigB), encoding an RNA polymerase-specific general (alternative) stress sigma factor (SigB) and having a nucleotide sequence which is at least 98%, and increasingly preferably 98.5, 99, 99.5 and 100%, identical to the nucleotide sequence set forth in SEQ ID NO. 123, in particular across the subregion corresponding to nucleotide positions 201 to 998 according to SEQ ID NO. 123.

Thioredoxin (TrxA), having an amino acid sequence which is at least 98.5%, and increasingly preferably 99, 99.5 and 100%, identical to the amino acid sequence set forth in SEQ ID NO. 126; in connection with a nucleic acid (trxA), encoding a thioredoxin (TrxA) and having a nucleotide sequence which is at least 93%, and increasingly preferably 94, 95, 96, 97, 98, 99 and 100%, identical to the nucleotide sequence set forth in SEQ ID NO. 125, in particular across the subregion corresponding to nucleotide positions 201 to 515 according to SEQ ID NO. 125.

Table 3 reveals the surprising observation that the sequences of these genes and proteins are, in most cases, not very identical, despite the close relationship of B. subtilis and B. licheniformis. In this connection, there are also within the genes in question regions which correspond to a higher degree and regions which correspond to a lower degree, as can be detected in each case by way of a generally known alignment.

One possible use, depicted in the present application, for each of said nucleotide sequences is that of a probe on chips for controlling biological processes, since, as described above, said genes are regarded as being representative in order to indicate the metabolic situation of an organism, in particular of a microorganism used for a fermentation. According to the previous comments, preference is given for this purpose to smaller regions of said genes, which are advantageously close to the 5′ end.

In this connection, it is possible to use the similarities of the particular sequences in order to detect comparable gene products across species boundaries. Probes to regions different from one another may be utilized in order to detect such mRNAs side-by-side by using one and the same chip, for example if one of said genes is expressed in cells of the second species or if the cultures are mixed cultures. This also enables contaminations, for example with representatives of the second species (or with other microorganisms, for example E. coli via the probes set out above), to be detected. This is particularly important for purity control, for example in a fermentation.

Another possible industrial use is that of specifically inactivating the genes in question, for example via homologous recombination, in strains which are utilized for synthesizing other compounds or in which the genes in question are to be specifically switched off, in order to provide in trans a homologous gene, for example a gene coding for a more active product.

In addition, the particular enzymes indicated under the even-numbered entries in the sequence listing are capable of the particular biochemical reactions which correspond to their role in the particular metabolic pathway. Accordingly, they may be used for carrying out comparable reactions in vitro. Thus, enzymes are increasingly used as catalysts, in particular for synthesis of natural substances such as vitamins, antibiotics or else of medicaments. Compared to conventional processes, they are distinguished in particular by usually lower temperatures, good environmental compatibility and high regioselectivity.

This applies in particular to products which correspond to the metabolic products located on said pathways in vivo. These are, for example, the transcriptional repressors CodY and GlnR for regulating the pathways in question, the fatty acid metabolism desaturase (Des) and the nucleotide metabolism factors PurC, PurN and PyrP.

The enzymes employed for these purposes may advantageously be obtained from the corresponding DNA sequences indicated under the odd numbers in the sequence listing by isolating or synthesizing the genes in question from B. licheniformis DSM 13 or comparable strains in a manner known per se and introducing said genes into expression vectors. It is also possible to let microorganisms which have obtained the activities in question in this manner catalyze the chemical reaction of interest.

Further aspects of the present invention and preferred embodiments will be illustrated by the following examples or evolve from the latter themselves.

### EXAMPLES

All molecular-biological steps follow standard methods as indicated, for example, in the manual by Fritsch, Sambrook and Maniatis “Molecular cloning: a laboratory manual”, Cold Spring Harbor Laboratory Press, New York, 1989, or comparable specialist works. Enzymes and kits were used according to the information of the particular manufacturer.

### Example 1

### Analysis of Expression of the Genes ibpB and dnaK of the Gram-Negative Bacterium Escherichia coli

The ibpB and dnak mRNA levels before and after overproduction of an insoluble model protein (Saccharomyces cerevisiae α-glycosidase) in Escherichia coli were determined using slot-blot analysis. The E. coli RB791 strain [F⁻, IN(rrnD-rrnE)1, λ⁻, lacl^{q}L₈] was used for the experiments. This strain harbors the pKK177glucC plasmid containing the α-glucosidase gene whose expression is induced by the tac promoter and addition of isopropyl-β-D-thiogalactopyranoside (IPTG). Said strain furthermore carries the pUBS520 plasmid which constitutively expresses a minor argU tRNA (Brinkmann et al., 1989).

Cultivation was carried out as fed batch fermentation in a 6-1 Biostat ED fermenter (B. Braun Biotech. Int., Melsungen, Germany). All fermentations were carried out in a glucose-ammonium-based mineral salt medium at a temperature of 35° C., as described in Teich et al. (1998: J. Biotechnol., vol. 8, pp. 197-210). The induction was carried out by adding 1 mM IPTG.

For mRNA analysis, the cells were taken up in 400 μl (1:1 (v/v)) killing buffer (20 mM Tris-HCl pH 7.5, 20 mM NaN₃, 5 mM MgCl₂) and centrifuged. The supernatant was discarded and the pellet stored at −80° C. until further analysis. Total RNA was isolated using the High Pure RNA isolation kit (Roche Diagnostics). Specified amounts of the isolated RNA were diluted with 10×SSC (1.5 M NaCl, 0.15 M sodium citrate, pH 7) and applied to a positively charged nylon membrane. This was followed by hybridization with a digoxigenin-labeled specific RNA probe according to the instructions in the Roche Diagnostics manual. The RNA probes were synthesized by T7 RNA polymerase in vitro from a PCR product which contained a T7 promoter sequence. The following primers were used for synthesizing the corresponding PCR products:

5′GCTTTACCGTTCTGCTATTGG (SEQ ID NO:127) and

5′CTAATACGACTCACTATAGGGAGAAGTTGATTTCGATACGGCGC (SEQ ID NO:128) for ibpB (cf. Allen et al., 1992; J. Bacteriol., vol. 174, pp. 6938-6947), and

5′GGGTAAAATAATGGTATCG (SEQ ID NO:129) and

5′CTAATACGACTCACTATAGGGAGACTTTGATGTTCATGTGTTTC (SEQ ID NO: 130) for dnaK (Bardwell and Craig, 1984; Proc. Natl. Acad. Sci., vol. 81, pp. 848-852).

The hybridization signals on the filter were quantified using the Roche Diagnostics Lumi imager (FIG. 1).

Maximum expression of both ibpB and dnaK is visible 1 h after induction of the expression system and, connected therewith, the formation of protein aggregates (inclusion bodies). Both chaperones are obviously required at this time. Conversely, both genes may also be regarded as markers for this special physiological state of E. coli.

### Example 2

### Analysis of Expression of the acoA Gene of the Gram-Positive Bacterium Bacillus subtilis

To analyze the acoA mRNA levels before and after glucose limitation, Northern blot analysis and realtime (RT) PCR with the aid of a LightCycler (Roche Diagnostics) were used according to the manufacturer's information. The PCR primers for this analysis should have properties (GC content, melting point, etc.) similar to one another. The size of the corresponding PCR product should be between 300 and 750 bp (optimally: 500 bp). The primer sequences were deduced using the “Primer 3” computer program. This program is freely available on the world wide web at genome.wi.mit.edu/cgi-bin/primer/primer3.cgi, or at genome.wi.mit.edu/cgi-bin/primer/primer3_www.cgi (last update: Sep. 9, 2002). The exemplary procedure is also described in the article “Quantification of Bacterial mRNA by One-Step RT-PCR using the LightCycler System” (2003) by S. Tobisch, T. Koburger, B. Jürgen, S. Leja, M. Hecker and T. Schweder in BIOCHEMICA, volume 3, pages 5 to 8.

The strain used for this experiment is Bacillus subtilis 168. The cells were cultured in minimal medium (Stülke et al., 1993; J. Gen. Microbiol., vol. 139, pp. 2041-2045). The main culture was inoculated with an overnight culture so as to achieve a starting OD_{540 nm} of 0.05. The culture was incubated in a small fermenter (working volume: 500 ml) at 37° C. The first sampling for RNA analysis was carried out at an OD of 0.4 (corresponding to a blank sample in the exponential growth phase or before stress). The sample volume is set here to exactly OD 16 (e.g. OD₅₀₀=0.4→16:0.4=40 ml to harvest). The second sample for RNA isolation was taken 30 min, 1 h and 2 h after transition to the stationary growth phase.

The cells were disrupted using a RiboLyser apparatus (ThermoLifeSciences) by mechanically destroying the cells by means of the glass beads present in the reaction vessel (glass pearls 0.1-0.11 mm Ø, B. Braun Biotech), caused by the rotating motions of the RiboLyser. In order to avoid RNAse activity, acidic phenol is added to the reaction mixture. After the cells have been disrupted, the reaction vessels are placed on ice in order to let them cool off a little.

The RNA was isolated and purified using the KingFisher apparatus (ThermoLifeSciences). The KingFisher is an automated pipetter. Biological substances bound to magnetic particles are transferred by means of bar magnets into various reaction vessels. Total RNA is isolated from the lysed cells by utilizing the KingFisher in combination with the MagNA Pure LC RNA isolation kit I (Roche Diagnostics). The apparatus is operated at 4° C.

The quantities of acoA mRNA before and after stress were analyzed with the aid of Northern blot analyses following standard protocols. The result is depicted in FIG. 2.

Table 4: Expression levels of the acoA gene at various points in time during B. subtilis growth, determined by way of quantification of the signals of the Northern blot analysis of FIG. 2

| Sample | Amount of mRNA obtained |
|---|---|
| [according to FIG. 2] | [in BLU] |
| 1 | 6.0 * 10³ |
| 2 | 8.7 * 10³ |
| 3 | 4.9 * 10⁵ |
| 4 | 1.1 * 10⁵ |
| 5 | 2.0 * 10⁴ |

It is obvious that the acoA gene is maximally expressed in the early stationary phase, i.e. that here, in the present example, glucose limitation has occurred. Said gene may thus be regarded as a marker gene for this special physiological state of B. subtilis, or an inventive probe for this gene should be able to indicate this state.

### Example 3

### Analysis of mRNA Levels of the Genes dnaK and sigB of the Gram-Positive Bacterium Bacillus licheniformis During Heat Shock

The amounts of dnaK and gsiB mRNA of B. licheniformis cells during a heat shock were determined by means of Northern blot analysis. For this purpose, cells of the B. licheniformis DSM16 strain were cultured in LB medium at 37° C. A preculture in the logarithmic growth state was used to inoculate the fermenter culture so as to achieve an OD₅₀₀ of approx. 0.05. The first sample for RNA isolation was taken at an OD of 0.4. This sample represents the control. Heat stress was carried out at 54° C. for 10 minutes. Subsequently, another cell sample for RNA isolation was removed.

The cells of both samples (control, stress) were again disrupted by means of the RiboLyser apparatus (see above). Total RNA was isolated from the lysate with the aid of the KingFisher apparatus (see above) and the MagNA Pure LC RNA isolation kit I (see above).

Specified amounts of the isolated RNA were fractionated again according to standard methods via gel electrophoresis and hybridized as above with a digoxigenin-labeled specific DNA probe according to the manufacturer's information. The probes for detecting the particular mRNA were synthesized according to standard methods by T7 RNA polymerase from PCR products of said genes which had been cloned in each case into vectors with T7 promoter. The result of the hybridizations with the dnaK- and sigB-specific probes is depicted in FIGS. 3A and B, respectively.

Both probes for the heat shock situation are seen producing a signal which is above that of the control, with the dnaK probe delivering a particularly clear result compared with the control.

### Example 4

### Analysis of Expression of the acoA Gene of the Gram-Positive Bacterium Bacillus subtilis via DNA Chips of the Invention

Similarly to example 2, the acoA mRNA level in the course of a fermentation was studied here, that is by using DNA chips of the invention and, for comparison, again via realtime RT-PCR. For this purpose, as in example 2, B. subtilis 168 was cultured in minimal medium in a 500-ml fermenter at 37° C. Samples for RNA analysis were taken at regular time intervals, the cells were disrupted in the manner described using the RiboLyser apparatus (ThermoLifeSciences), and the RNA (in each case 10 μg of total RNA) was again isolated and purified using the KingFisher apparatus (ThermoLifeSciences). The acoA mRNA-containing samples were then quantified in two different ways: (A), as described in example 2, via realtime RT-PCR with the aid of the LightCycler (Roche Diagnostics) and (B) via chips of the invention doped with probes for said gene.

This chip had been constructed as described in the article by Hintsche et al. (1997), EXS, 80, pp. 267-283 and the applications WO 00/62048 A2, WO 00/67026 A1 and WO 02/41992 (see above). The DNA probe for detecting the acoA mRNA was 20 nucleotides in length and derived from a region close to the start of the coding region according to SEQ ID NO. 1.

The result is depicted in FIG. 5. The curve profile in FIG. 5A, which represents the relative absorptions determined by means of the RT-PCR LightCycler, reveals that the gene induced with glucose deficiency (compare example 2) is increasingly expressed up to time point 5, whereafter the level of the corresponding mRNA decreases again slightly in order to reach a distinctly higher maximum toward the end, at time point 8. Thus, toward the end of the fermentation, the carbon sources of the fermentation medium were already strongly depleted so that the cells encountered glucose deficiency. However, as the error bars in FIG. 5A indicate, these data have a wide range of fluctuation.

FIG. 5B which depicts the course of the electrical signals obtained via an electrochip of the invention and indicated in nA shows the same curve profile in principle, in particular the strong rise in glucose limitation toward the end of the fermentation. The intermediate maximum after 5 h, which is visible in A, is not evident here, but the error bars in figure A also allow for the fact that a maximum was actually not present here.

Comparison of the two curves teaches that both measuring techniques deliver the same results in principle. The chip of the invention, however, has a distinct advantage in the substantially smaller range of fluctuation of the data obtained.

### Example 5

### Monitoring the aprE Product Gene by Means of RT-PCR and an Electrical Chip of the Invention During Fermentation of B. licheniformis DSM 13

In this example, the course of expression of the aprE gene by Bacillus licheniformis DSM13 is studied. The gene in this case is the gene which naturally encodes an extracellular alkaline protease of said strain (subtilisin E) and which is induced in vivo during the stationary growth phase. This analysis corresponds with respect to the application of the present invention to the monitoring of a product gene of interest during the fermentative preparation of the protein of interest by such a microorganism.

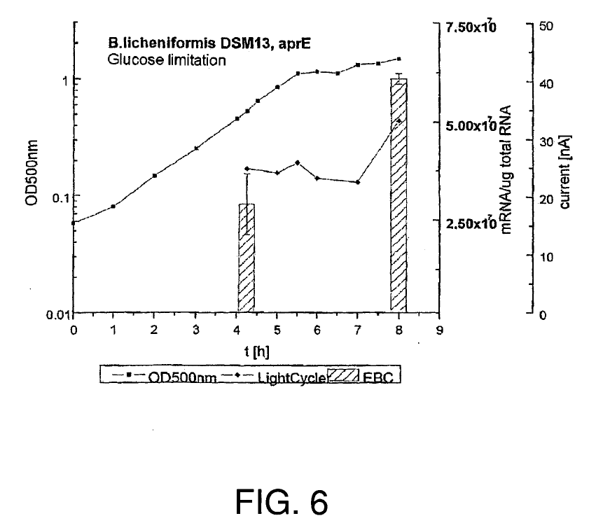

To this end, cells of the strain B. licheniformis DSM13 were cultured in minimal medium (Stülke et al., (1993), J. Gen. Microbiol., volume 139, pages 2041-2045). The main culture was inoculated with an overnight culture so as to obtain a starting OD at 540 nm of 0.05. The cultures were incubated in each case in 1 l of medium in 5-1 shaker flasks at 37° C. and samples were taken at the times indicated in FIG. 6. Said samples were worked up as described in examples 2 and 4, and the mRNA coding for the alkaline protease AprE was detected both via an RT-PCR LightCycler and via an electrical DNA chip of the invention. The latter was doped with a probe specific for said gene, which probe was 20 nucleotides in length and had been derived from a region close to the start of the coding region of the corresponding known B. licheniformis gene.

The result is depicted in FIG. 6. This reveals the cell density, indicated as optical density at 500 nm (OD500 nm), the share of the specific mRNA determined via the LightCycler apparatus in the total RNA, indicated in molecules per μg (LightCycler), and the signals, determined at two points in time, of the electrical biochip doped with the aprE probe in nA (EBC).

The data indicate that expression of the aprE gene is detectable after 4 h, i.e. at the start of the stationary phase, and then increases. This observation correlates with the known regulation of the gene by a stationary phase promoter.

### Example 6

### Monitoring of the Phosphate Deficiency-Indicating Gene pstS by Means of RT-PCR and an Electrical Chip of the Invention During Fermentation of B. licheniformis DSM 13

In this example, the course of expression of the pstS gene by Bacillus licheniformis DSM13 is studied. The gene in this case, as depicted in table 2, is the gene which encodes a phosphate-binding protein and which is induced by said strain in vivo during phosphate starvation. This analysis corresponds with respect to the application of the present invention to the monitoring of a corresponding stress signal during fermentation of such a microorganism.

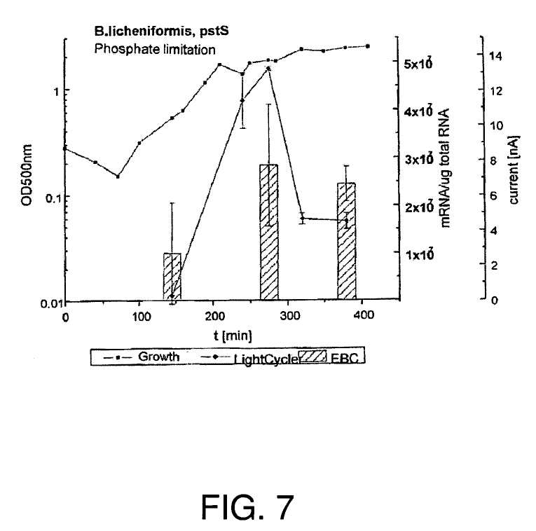

To this end, cells of the B. licheniformis DSM13 strain were cultured in minimal medium, similarly as in example 5. The main culture was inoculated with an overnight culture so as to obtain a starting OD at 540 nm of 0.05. The cultures were incubated in each case in 500 ml of medium in a Biostat Q fermenter from Braun Biotech International (Melsungen, Germany) at 37° C. At one point during the exponential growth phase (0 min in FIG. 7), 1.5 μM KH₂PO₄ were added to the medium. This results in a state of phosphate deficiency which should affect expression of the pstS gene.

Samples were taken, as described in examples 2 and 4, at the times indicated in FIG. 7, said samples were worked up and the mRNA coding for the phosphate-binding protein PstS was detected both via an RT-PCR LightCycler and via an electrical DNA chip of the invention. The latter was doped with a probe specific for said gene, which probe was 20 nucleotides in length and had been derived from a region close to the start of the coding region of the DNA sequence listed under SEQ ID NO. 115.

The result is depicted in FIG. 7. This reveals the cell density, indicated as optical density at 500 nm (OD500 nm), the share of the specific mRNA determined via the LightCycler apparatus in the total RNA, indicated in molecules per μg (LightCycler), and the signals, determined at three points in time, of the electrical biochip doped with a pstS probe in nA (EBC).

The data show a decrease in cell density immediately after the onset of phosphate deficiency and a recovery of bacterial growth after approx. 100 to 150 min. This correlates with expression of the pstS gene, which is detectable by both methods of measurement. Thus it is possible according to the invention to use an appropriately doped chip for the purpose of recording a phosphate deficiency situation.

### Example 7

### Monitoring of the Glucose Limitation-Indicating acoA Gene by Means of RT-PCR and an Electrical Chip of the Invention During Fermentation of B. licheniformis DSM 13

In this example, the course of expression of the acoA gene by Bacillus licheniformis DSM13 is studied. The gene here is, as depicted in table 2, the gene encoding the acetoin dehydrogenase E1 component (TPP-dependent α subunit; E.C. 1.2.4.-), which gene is induced by this strain in vivo with glucose limitation. Like example 6, this analysis corresponds with respect to the application of the present invention to the monitoring of a corresponding stress signal during fermentation of such a microorganism.

To this end, cells of the B. licheniformis DSM13 strain were cultured in minimal medium, similarly as in examples 5 and 6. The main culture was inoculated with an overnight culture so as to obtain a starting OD at 540 nm of 0.05. The cultures were incubated in each case in 500 ml of medium in a Biostat Q fermenter from Braun Biotech International (Melsungen, Germany) at 37° C. However, said medium contained the small amount of 0.05% by weight of glucose so that glucose deficiency is established comparatively early, already during the exponential growth phase, which glucose deficiency should affect expression of the acoA gene.

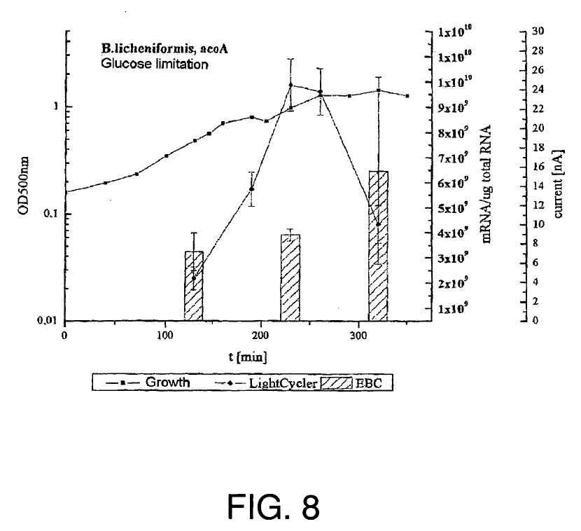

Samples were taken, as described in examples 2 and 4, at the times indicated in FIG. 8, said samples were worked up and the mRNA coding for the acetoin dehydrogenase E1 component AcoA was detected both via an RT-PCR LightCycler and via an electrical DNA chip of the invention. The latter was doped with a probe specific for said gene, which probe was 20 nucleotides in length and had been derived from a region close to the start of the coding region of the DNA sequence listed under SEQ ID NO. 83.

The result is depicted in FIG. 8. This reveals the cell density, indicated as optical density at 500 nm (OD500 nm), the share of the specific mRNA determined via the LightCycler apparatus in the total RNA, indicated in molecules per μg (LightCycler), and the signals, determined at three points in time, of the electrical biochip doped with the pstS probe in nA (EBC).

The data indicate a more restrained growth than in FIG. 6, for example, and the onset of expression of the acoA marker gene as a response to the glucose deficiency situation after approx. 130 min. Thus it is possible according to the invention to use an appropriately doped chip for the purpose of recording a glucose deficiency situation.

### Example 8

### Exemplary Charging with a Producer Organism of the Genus Bacillus for a Fermentation for Protease Production

Similarly to the preceding examples, an inventive chip for recording a fermentation of a producer organism of the genus Bacillus, established for industrial fermentations, is doped simultaneously with a plurality of probes, that is probes for the following genes (table 5. part 1):

| | | | Corresponding | Corresponding |
|---|---|---|---|---|
| | | | B. subtilis | B. licheniformis |
| No. | Gene | Function | sequence | sequence |
| 1 | groEL | Chaperonin | SEQ ID NO. 33 | SEQ ID NO. 107 |
| 2 | clpC | Stress response | SEQ ID NO. 9 | SEQ ID NO. 91 |
| 3 | phoD | Phosphate | SEQ ID NO. 59 | |
| | | starvation | | |
| 4 | purN | Purine synthesis | SEQ ID NO. 67 | SEQ ID NO. 119 |
| 5 | pyrB | Pyrimidine | SEQ ID NO. 69 | |
| | | synthesis | | |
| 6 | trxA | Oxidative stress | SEQ ID NO. 77 | SEQ ID NO. 125 |
| 7 | cspA | Stationary phase | SEQ ID NO. 17 | |
| | | | (from E. coli) | |

These probes are advantageously derived from the sequences cited here and indicated in the sequence listing, according to the information given in the description.

In this example, the fermentative production of a protease is monitored, similarly to example 5. For this purpose, an appropriate chip is additionally equipped with probes for the following genes (table 5, part 2):

| | | | Corresponding | Corresponding |
|---|---|---|---|---|
| | | | B. subtilis | B. licheniformis |
| No. | Gene | Function | sequence | sequence |
| 8 | | Protease gene | | |
| | | (product gene) | | |
| 9 | tnrA | Nitrogen | SEQ ID NO. 75 | |
| | | metabolism | | |
| 10 | codY | Nitrogen | SEQ ID NO. 15 | SEQ ID NO. 95 |
| | | metabolism | | |
| 11 | glnR | Nitrogen | SEQ ID NO. 31 | SEQ ID NO. 105 |
| | | metabolism | | |

In this case, observation of the nitrogen metabolism, which supplements the growth data (Nos. 1 to 7), is useful because formation of the preferably overexpressed gene of interest affects the utilization of nitrogen sources and, accordingly, nitrogen metabolism is stimulated in the course of a successful fermentation. This supplements or replaces fermentation-accompanying assays for enzyme activity.

### Example 9

### Exemplary Charging with a Producer Organism of the Genus Bacillus for a Fermentation for Amylase Production

A fermentation for producing an amylase by a producer organism of the genus Bacillus is observed by doping a chip of the invention with probes for the same genes as indicated in table 5, part 1. In this example, fermentative production of an amylase is monitored, similarly to example 8. For this purpose, an appropriate chip is additionally equipped with probes for the following genes (table 6):

| | | | Corresponding | Corresponding |
|---|---|---|---|---|
| | | | B. subtilis | B. licheniformis |
| No. | Gene | Function | sequence | sequence |
| 8 | | Amylase gene | | |
| | | (product gene) | | |
| 9 | acoA | Glucose limitation | SEQ ID NO. 1 | SEQ ID NO. 83 |
| 10 | eno | Glucose starvation | SEQ ID NO. 29 | SEQ ID NO. 103 |
| 11 | citB | Active citrate cycle | SEQ ID NO. 7 | SEQ ID NO. 89 |

In this case, observation of the carbon metabolism, which supplements the growth data (Nos. 1 to 7), is useful because formation of the preferably overexpressed gene of interest affects the utilization of carbon sources and, accordingly, the glucose metabolism is stimulated in the course of a successful fermentation. This supplements or replaces fermentation-accompanying assays for enzyme activity.

Description of the Figures

FIG. 1: ibpB and dnaK as marker genes of the Gram-negative bacterium Escherichia coli for the formation of protein aggregates (inclusion bodies; cf. example 1); induction of the expression system at 0 h; protein aggregates form from this time onward.

A: Expression of the ibpB gene; determined via isolating the mRNA at the time in question, binding to a nylon membrane and hybridization with an ibpB-specific digoxigenin-labeled probe.

B: Expression of the dnaK gene; determined analogously to A.

FIG. 2: The acoA gene as a marker gene of the Gram-positive bacterium Bacillus subtilis for glucose limitation and presence of acetoin, determined via Northern blot analysis with an acoA probe (cf. example 2).

A: RNA gel whose lanes are occupied as follows:

1. early logarithmic phase (OD₅₀₀=0.4)

2. transient phase (transition from exponential phase to stationary phase)

3. early stationary phase (45 min after the end of logarithmic phase)

4. late stationary phase (180 min after the end of logarithmic phase)

5. recovery after addition of glucose

B: Northern blot of the gel depicted in A with an acoA probe

FIG. 3: The genes dnaK and sigB as marker genes of the Gram-positive bacterium Bacillus licheniformis for heat shock, determined via Northern blot analysis with dnaK and sigB probes, respectively (cf. example 3).

A: Northern blot, assayed with dnaK probe; lanes are occupied as follows:

M marker

Co control

54° C. after 54° C. heat shock

Co control

54° C. after 54° C. heat shock

B: Northern blot, assayed with sigB probe; lanes occupied as in A.

FIG. 4: Diagrammatic representation of at line monitoring of a bioprocess by means of electrical DNA chips of the invention. Said bioprocess is advantageously monitored close to real time via the following steps; additionally indicated is the approximate time needed in each case:

1. sampling (a few seconds);

2. cell disruption via routine methods (approx. 5 min);

3. RNA isolation via routine methods (approx. 20 min);

4. hybridization on a chip loaded according to the invention with nucleic acids (e.g. DNA) or nucleic acid analogs (e.g. similarly constructed, difficult-to-hydrolyze compounds);

5. recording the electrical signals of a correspondingly constructed electrochip; alternatively, it would also be possible to record optical signals of an optical DNA chip;

6. preferably computer-assisted data evaluation (a few minutes).

Using electrical chips produces an approximate total time of analysis of approx. 2 to 3 h, using conventional optical DNA chips produces a time of approx. 12 h.

FIG. 5: Comparison of measurement sensitivity when using electrical DNA chips, in comparison with RT-PCR

As described in example 4, the same total RNA preparation of fed batch fermentation of B. subtilis was studied with respect to the acoA gene.

A: Measuring the relative absorptions in an RT-PCR LightCycler;

B: Measuring the electrical signals (in nA) via an electrochip of the invention.

The, in principle, identical curve profile, but a substantially smaller fluctuation range, are clearly visible when using a chip of the invention.

FIG. 6: Monitoring of the aprE product gene by means of RT-PCR and an electrical chip of the invention during fermentation of B. licheniformis DSM 13 according to example 5.

In this connection:

OD500 nm: is cell density;

LightCycler: is the share of the specific mRNA determined for six time points via the LightCycler apparatus in total RNA, indicated in molecules per μg;

EBC: is the signals of the electrical biochip doped with a aprE probe, determined at two time points and in measured nA.

FIG. 7: Monitoring of the phosphate deficiency-indicating pstS gene by means of RT-PCR and an electrical chip of the invention during fermentation of B. licheniformis DSM 13 according to example 6.

In this connection:

OD500 nm: is cell density;

LightCycler: is the share of the specific mRNA determined for five time points via the LightCycler apparatus in total RNA, indicated in molecules per μg;

EBC: is the signals of the electrical biochip doped with an pstS probe, determined at three time points and in measured nA.

FIG. 8:b Monitoring of the glucose limitation-indicating acoA gene by means of RT-PCR and an electrical chip of the invention during fermentation of B. licheniformis DSM 13 according to example 7.

In this connection:

OD500 nm: is cell density;

LightCycler: is the share of the specific mRNA determined for five time points via the LightCycler apparatus in total RNA, indicated in molecules per μg;

EBC: is the signals of the electrical biochip doped with an acoA probe, determined at three time points and in measured nA.

1. A method for determining the physiological state of cells that are undergoing a biological process comprising
providing a chip comprising a solid support to which probes comprising at least a portion of the coding region of at least four of the following genes are attached: acoA, ahpC, ahpF, citB, clpC, clpP, codY, cspA, cspB, des, dnaK, eno, glnR, groEL, groL, gsiB, ibpA, ibpB, katA, katE, IctP, Idh, opuAB, phoA, phoD, pstS, purC, purN, pyrB, pyrP, sigB, tnrA, trxA, and ydjF, or genes that are regulated identically to the acoA, ahpC, ahpF, citB, clpC, clpP, codY, cspA, cspB, des, dnaK, eno, glnR, groEL, groL, gsiB, ibpA, ibpB, katA, katE, IctP, Idh, opuAB, phoA, phoD, pstS, purC, purN, pyrB, pyrP, sigB, tnrA, trxA, and ydjF genes;
contacting the chip with mRNA isolated from the cells; and
detecting mRNA that specifically binds to at least one of the probes.

2. (canceled)

3. The method of claim 1 wherein the biological process is a fermentation.

4. The method of claim 3 wherein the cells contain an exogenous gene that encodes a gene product of interest and the exogenous gene is expressed during the fermentation.

5. The method of claim 4 wherein the gene product of interest is an amylase, a cellulase, a lipase, an oxidoreductase or a protease.

6. The method of claim 4 wherein at least one probe comprises at least a portion of the coding region of the exogenous gene.

7. The method of claim 1 wherein the probes are single stranded DNA or single stranded DNA analogs.

8. The method of claim 1 wherein each probe comprises at least a portion of the coding region of one gene and the probes collectively comprise a portion of the coding region of 6, 8, 10, 12, 14, 16, 18, 20, 22, 24, 26, 28, 30, 32, or 34 genes.

9. The method of claim 1 wherein the probes comprise at least a portion of the coding region of at least one of the following genes: clpP, cspA, cspB, dnaK, groL, ibpA, ibpB, katE, phoa, pstS, and trxA, or genes that are regulated identically to the clpP, cspA, cspB, dnaK, groL, ibpA, ibpB, katE, phoa, pstS, and trxA genes.

10. The method of claim 1 wherein the organism of interest is a unicellular eukaryote, gram-positive bacteria, or gram-negative bacteria.

11. The method of claim 1 wherein the organism of interest is of the genera Sacharomyces or Schizosaccharomyces.

12. The method of claim 1 wherein the organism of interest is Bacillus subtilis, Bacillus amyloliquefaciens, Bacillus licheniformis, Bacillus agaradherens, Bacillus stearothermophilus, Bacillus lentus, or Bacillus globigii.

13. The method of claim 12 wherein the probes comprise at least a portion of the coding region of at least one of the following genes: acoA, ahpC, ahpF, citB, clpC, clpP, codY, cspB, des, dnaK, eno, glnR, groEL, gsiB, katA, katE, IctP, Idh, opuAB, phoA, phoD, pstS, purC, purN, pyrB, pyrP, sigB, tnrA, trxA, and ydjF.

14. The method of claim 1 wherein the organism of interest is of the genera Escherichia coli or Klebsiella.

15. The method of claim 14 wherein the organism of interest is of the genus Escherichia coli and the probes comprise at least a portion of the coding region of at least one of the following genes: clpP, cspA, cspB, dnaK, groL, ibpA, ibpB, katE, phoA, pstS, and trxA.

16. The method of claim 1 wherein mRNA that specifically binds to at least one of the probes is detected using an electrical signal.

17. A chip comprising a solid support to which probes comprising at least a portion of the coding region of at least four of the following genes are attached: acoA, ahpC, ahpF, citB, clpC, clpP, codY, cspA, cspB, des, dnaK, eno, glnR, groEL, groL, gsiB, ibpA, ibpB, katA, katE, IctP, Idh, opuAB, phoA, phoD, pstS, purC, purN, pyrB, pyrP, sigB, tnrA, trxA, and ydjF, or genes that are regulated identically to the acoA, ahpC, ahpF, citB, clpC, clpP, codY, cspA, cspB, des, dnaK, eno, glnR, groEL, groL, gsiB, ibpA, ibpB, katA, katE, IctP, Idh, opuAB, phoA, phoD, pstS, purC, purN, pyrB, pyrP, sigB, tnrA, trxA, and ydjF genes.

18. The chip of claim 17 wherein the probes are single stranded DNA or single stranded DNA analogs.

19. A polypeptide which is:
an alpha subunit of the acetoin dehydrogenase E1 component comprising an amino acid sequence that is at least 74% identical to the amino acid sequence set forth in SEQ ID NO:84;
a small subunit of alkyl hydroperoxide reductase comprising an amino acid sequence that is at least 95% identical to the amino acid sequence set forth in SEQ ID NO:86;
a large subunit of alkyl hydroperoxide reductase/NADH dehydrogenase comprising an amino acid sequence that is at least 92% identical to the amino acid sequence set forth in SEQ ID NO:88;
an aconitase hydratase comprising an amino acid sequence that is at least 93% identical to the amino acid sequence set forth in SEQ ID NO:90;
a class III stress response-related ATPase comprising an amino acid sequence that is at least 95% identical to the amino acid sequence set forth in SEQ ID NO:92;
a proteolytic subunit of the ATP-dependent protease comprising an amino acid sequence that is at least 97% identical to the amino acid sequence set forth in SEQ ID NO:94;
a pleiotropic transcriptional repressor comprising an amino acid sequence that is at least 92% identical to the amino acid sequence set forth in SEQ ID NO:96;
a major cold shock protein comprising an amino acid sequence that is at least 98% identical to the amino acid sequence set forth in SEQ ID NO:98;
a fatty acid desaturase comprising an amino acid sequence that is at least 73% identical to the amino acid sequence set forth in SEQ ID NO:100;
a class I heat shock protein comprising an amino acid sequence that is at least 94% identical to amino acids 1 to 480 of SEQ ID NO:102;
an enolase comprising an amino acid sequence that is at least 98% identical to the amino acid sequence set forth in SEQ ID NO:104;
a transcriptional repressor of the glutamine synthetase gene comprising an amino acid sequence that is at least 83% identical to the amino acid sequence set forth in SEQ ID NO:106;
a class I heat shock protein comprising an amino acid sequence that is at least 96% identical to the amino acid sequence set forth in SEQ ID NO:108;
a catalase comprising an amino acid sequence that is at least 90% identical to the amino acid sequence set forth in SEQ ID NO:110;
a catalase comprising an amino acid sequence that is at least 74% identical to the amino acid sequence set forth in SEQ ID NO:112;
a glycine-betaine ABC transporter comprising an amino acid sequence that is at least 82% identical to the amino acid sequence set forth in SEQ ID NO:114;
a phosphate-binding protein comprising an amino acid sequence that is at least 82% identical to the amino acid sequence set forth in SEQ ID NO:116;
a phosphoribosylaminoimidazole succinocarboxamide synthetase comprising an amino acid sequence that is at least 90% identical to the amino acid sequence set forth in SEQ ID NO:118;
a phosphoribosylglycinamide formyltransferase comprising an amino acid sequence that is at least 76% identical to the amino acid sequence set forth in SEQ ID NO:120;
a uracil permease comprising an amino acid sequence that is at least 69% identical to the amino acid sequence set forth in SEQ ID NO:122;
an RNA polymerase-specific general stress sigma factor comprising an amino acid sequence that is at least 85% identical to the amino acid sequence set forth in SEQ ID NO:124; or
a thioredoxin comprising an amino acid sequence that is at least 98.5% identical to the amino acid sequence set forth in SEQ ID NO:126.

20. A nucleic acid which is:
a nucleic acid encoding an alpha subunit of the acetoin dehydrogenase E1 component comprising a nucleotide sequence that is at least 85% identical to nucleotides 201 to 877 of SEQ ID NO:83;
a nucleic acid encoding a small subunit of alkyl hydroperoxide reductase comprising a nucleotide sequence that is at least 91% identical to nucleotides 201 to 764 of SEQ ID NO:85;
a nucleic acid encoding a large subunit of alkyl hydroperoxide reductase/NADH dehydrogenase comprising a nucleotide sequence that is at least 87% identical to nucleotides 201 to 1730 of SEQ ID NO:87;
a nucleic acid encoding an aconitase hydratase comprising a nucleotide sequence that is at least 93% identical to nucleotides 201 to 2927 of SEQ ID NO:89;
a nucleic acid encoding a class III stress response-related ATPase comprising a nucleotide sequence that is at least 84% identical to nucleotides 201 to 2633 of SEQ ID NO:91;
a nucleic acid encoding a proteolytic subunit of the ATP-dependent protease comprising a nucleotide sequence that is at least 86% identical to nucleotides 1 to 549 of SEQ ID NO:93;
a nucleic acid encoding a pleiotropic transcriptional repressor comprising a nucleotide sequence that is at least 88% identical to nucleotides 201 to 980 of SEQ ID NO:95;
a nucleic acid encoding a major cold shock protein comprising a nucleotide sequence that is at least 97% identical to nucleotides 201 to 401 of SEQ ID NO:97;
a nucleic acid encoding a fatty acid desaturase comprising a nucleotide sequence that is at least 88% identical to nucleotides 201 to 1229 of SEQ ID NO:99;
a nucleic acid encoding a class I heat shock protein comprising a nucleotide sequence that is at least 88% identical to nucleotides 1 to 1440 of SEQ ID NO:101;
a nucleic acid encoding an enolase comprising a nucleotide sequence that is at least 94% identical to nucleotides 201 to 1493 of SEQ ID NO:103;
a nucleic acid encoding a transcriptional repressor of the glutamine synthetase gene comprising a nucleotide sequence that is at least 91% identical to nucleotides 201 to 608 of SEQ ID NO:105;
a nucleic acid encoding a class I heat shock protein comprising a nucleotide sequence that is at least 90% identical to nucleotides 201 to 1835 of SEQ ID NO:107;
a nucleic acid encoding a catalase comprising a nucleotide sequence that is at least 86% identical to nucleotides 201 to 1661 of SEQ ID NO:109;
a nucleic acid encoding a catalase comprising a nucleotide sequence that is at least 85% identical to nucleotides 201 to 1661 of SEQ ID NO: 111;
a nucleic acid encoding a glycine-betaine ABC transporter comprising a nucleotide sequence that is at least 85% identical to nucleotides 201 to 1055 of SEQ ID NO:113;
a nucleic acid encoding a phosphate-binding protein comprising a nucleotide sequence that is at least 84% identical to nucleotides 201 to 1118 of SEQ ID NO:115;
a nucleic acid encoding a phosphoribosylaminoimidazole succinocarboxamide synthetase comprising a nucleotide sequence that is at least 89% identical to nucleotides 201 to 917 of SEQ ID NO:117;
a nucleic acid encoding a phosphoribosylglycinamide formyltransferase comprising a nucleotide sequence that is at least 84% identical to nucleotides 201 to 788 of SEQ ID NO:119;
a nucleic acid encoding a uracil permease comprising a nucleotide sequence that is at least 89% identical to nucleotides 201 to 1505 of SEQ ID NO:121;
a nucleic acid encoding an RNA polymerase-specific general stress sigma factor comprising a nucleotide sequence that is at least 98% identical to nucleotides 201 to 998 of SEQ ID NO:123; or
a nucleic acid encoding a thioredoxin comprising a nucleotide sequence that is at least 93% identical to nucleofides 201 to 515 of SEQ ID NO:125.

21. The method of claim 1 wherein the probes comprise at least a portion of the coding region of at least one of the following sequences: SEQ ID NO.1, 3, 5, 7, 9,11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, and 125.

22. The method of claim 1 wherein the probes are complementary to sequences near the 5′ end of mRNA molecules expressed in the cells.

23. The method of claim 1 wherein the probes are less than 100 nucleotides in length.

24. The method of claim 1 wherein the specific binding of mRNA isolated from the cells to a probe triggers an electrical signal.

25. The method of claim 12 wherein the probes comprise at least a portion of the coding region of at least one of the following sequences: SEQ ID NO. 1, 3, 5, 7, 9, 11, 15, 19, 23, 25, 29, 31, 33, 37, 43, 45, 49, 51, 53, 55, 59, 61, 65, 67, 69, 71, 73, 75, 77, 81, 83, 85, 87, 89, 91, 93, 95, 97, 99, 101, 103, 105, 107, 109, 111, 113, 115, 117, 119, 121, 123, and 125.

26. The method of claim 14 wherein the probes comprise at least a portion of the coding region of at least one of the following sequences: SEQ ID NO. 13, 17, 21, 27, 35, 39, 41, 47, 57, 63, and 79.

27. The chip of claim 17 wherein the probes are less than 100 nucleotides in length.
